SEQUENCE LISTING (3 sequences)

SEQ ID NO: 1 - DNA, 69 bp, Artificial Sequence
  Synthetic Template
ttttttttttaggaacccttggccaaatttttcccccggaaacagctatgaccggtcatagctgtttcc

SEQ ID NO: 2 - DNA, 13 bp, Artificial Sequence
  Synthetic Primer
cacattgtcaagg

SEQ ID NO: 3 - DNA, 49 bp, Artificial Sequence
  WT49G Synthetic Template
cagcttaagcaatggtacatgccttgacaatgtgtacatcaacatcacc

(19) United States
(12) Patent Application Publication
(10) Pub. No.: US 2018/0327828 A1
(43) Pub. Date: Nov. 15, 2018

(54) ION SENSOR DNA AND RNA SEQUENCING BY SYNTHESIS USING NUCLEOTIDE REVERSIBLE TERMINATORS

(71) Applicants: Jingyue Ju, Englewood Cliffs, NJ (US); Xiaoxu Li, New York, NY (US); Zengmin Li, Flushing, NY (US); Shiv Kumar, Belle Mead, NJ (US); Xin Chen, New York, NY (US); Cheng Guo, Brooklyn, NY (US); Shundi Shi, Ozone Park, NY (US); Jianyi Ren, New York, NY (US); Chuanjuan Tao, Fort Lee, NJ (US); Minchen Chien, Tenafly, NJ (US); James J. Russo, New York, NY (US); Lin Yu, Flushing, NY (US)

(72) Inventors: Jingyue Ju, Englewood Cliffs, NJ (US); Xiaoxu Li, New York, NY (US); Zengmin Li, Flushing, NY (US); Shiv Kumar, Belle Mead, NJ (US); Xin Chen, New York, NY (US); Cheng Guo, Brooklyn, NY (US); Shundi Shi, Ozone Park, NY (US); Jianyi Ren, New York, NY (US); Chuanjuan Tao, Fort Lee, NJ (US); Minchen Chien, Tenafly, NJ (US); James J. Russo, New York, NY (US); Lin Yu, Flushing, NY (US)

(73) Assignee: The Trustees of Columbia University in the City of New York, New York, NY (US)

(21) Appl. No.: 15/777,416

(22) PCT Filed: Nov. 18, 2016
(86) PCT No.: PCT/US2016/062917

§ 371 Date: May 18, 2018

Related U.S. Application Data
(60) Provisional application No. 62/257,147, filed on Nov. 18, 2015.

(51) Int. Cl. C12Q 1/6869

(52) U.S. Cl. CPC C12Q 1/6869

(57) ABSTRACT

This disclosure is related to a method for determining the identity of a nucleotide residue of a single-stranded DNA or RNA, or sequencing DNA or RNA, in a solution using an ion-sensing field effect transistor and reversible nucleotide terminators.

### CROSS-REFERENCES TO RELATED APPLICATIONS

This application claims the benefit of U.S. Provisional Application No. 62/257,147, filed Nov. 18, 2015, which is incorporated herein by reference in its entirety and for all purposes.

This invention was made with government support under grant nos. HG003582 and HG005109 awarded by the National Institutes of Health. The U.S. Government has certain rights in this invention.

### REFERENCE TO A SEQUENCE LISTING SUBMITTED AS AN ASCII FILE

The Sequence Listing written in an ASCII file-type, named “161118_88183-A-PCT_Sequence_Listing_RBR.txt”, which is 1 kilobyte in size, and which was created Nov. 18, 2016 in IBM-PCT machine format, having an operating system compatability with MS-Windows, and which is contained in the text file, filed Nov. 18, 2016 as part of this application

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

High-throughput sequencing has become a basic support technology for essentially all areas of modern biology, from arenas as disparate as ecology and evolution to gene discovery and personalized medicine. Through the use of massively parallel sequencing in all its varieties, it is possible to identify homology among genes throughout the tree of life, to detect single nucleotide polymorphisms (SNPs), copy number variants, and genomic rearrangements in individual humans; to characterize in detail the transcriptome and its transcription factor binding sites; and to provide a detailed and even global view of the epigenome (Hawkins et al. 2010; Morozova et al. 2009; Park et al. 2009).

In order to move the field of personalized medicine forward, it will be essential to garner complete genotype and phenotype information for representative samples of all geo-ethnic population groups, including individuals presenting with a broad range of complex diseases. Having such a compendium of data will eventually permit physicians to tailor treatment to each patient, taking into account genetic factors controlling their ability to tolerate and respond to different pharmaceuticals. This will require, however, the cost of whole genome sequencing to be in the range of most other medical tests, generally taken to be $1,000 or less, and to have a lower error rate per base than the frequency of all but the rarest SNPs (<1 in 10,000) (Fuller et al. 2009; Ng et al. 2010; Shen et al. 2010).

A variety of recent so-called “next generation” sequencing technologies have brought down the cost of sequencing a genome with relatively high accuracy close to $100,000, but this is still prohibitive for health care systems even in the most affluent countries. Further efficiencies in current technologies and the introduction of breakout technologies are required to move the field to the $1,000 goal. Among the “next generation” sequencing technologies, the most popular has been the sequencing by synthesis (SBS) strategy (Fuller et al. 2009) which underlies such diverse instruments as those commercialized or in development by companies such as Roche, Illumina, Helicos, and Intelligent BioSystems. One successful SBS approach involves the use of fluorescently labeled nucleotide reversible terminators (NRTs) (Ju et al. 2003; Li et al. 2003; Ruparel et al. 2005; Seo et al. 2005; Ju et al. 2006). These are modified dNTPs (A, C, T/U and G) that have both a base-specific fluorophore and a moiety blocking the 3′ hydroxyl group of the sugar and thereby impeding its extension by the next nucleotide attached to each dNTP via a chemically, enzymatically, or photo-cleavable bond. This permits one to interrupt the polymerase reaction, determine the base incorporated according to the color of the attached fluorescent tag, and then remove both the fluor and the 3′-OH blocking group, to permit one more base to be added. The importance of the use of NRTs is that they greatly reduce the possibility of read-ahead due to the addition of more than one nucleotide, especially with the use of intermediate synchronization strategies. Both Roche's pyrosequencing approach (Ronaghi et al. 1998) and Helicos' use of “virtual” terminators (Bowers et al. 2009; Harris et al. 2008) require the addition of each base, one by one, followed by a readout that is indirect (light production in the former), or direct but single color (in the latter). Despite the undeniable power of these methods (long read length for Roche, single molecule capability for Helicos), the methods have difficulty in accurately decoding homopolymer stretches longer than ˜4 or 5 bases (Ronaghi et al. 2001). Further, pyrosequencing suffers from false positives, as free dNTPs will spontaneously decompose in solution, releasing a pyrophosphate (Gerstein 2001), producing a signal.

A class of nucleotide analogues with unprotected 3′-OH and a cleavable disulfide linker attached between the base and fluorescent dye has been reported (Turcatti et al. 2008; Mitra et al. 2003). However, after DNA polymerase catalyzed extension reaction on the primer/template and imaging the incorporated base, the cleavage of the disulfide linkage generates a free reactive —SH group which has to be capped with alkylating agent, iodoacetamide as shown below, before the second extension reaction can be carried out. This capping step not only adds an extra step in the process but also limits the addition of multiple nucleotides in a row because of the long remnant tail on the nucleotide base moiety. With this approach the sequencing read length is limited to only 10 bases (Turcatti et al. 2008). Other disulfide based approaches require a similar capping reaction to render the free SH group unreactive (Mitra et al. 2003).

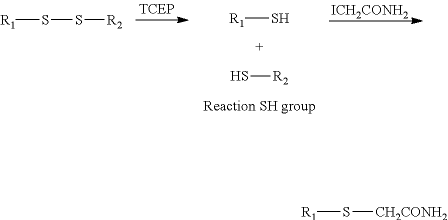

For the long read SBS strategy it is preferable that the cleavable linker is stable during the sequencing reactions, requires less manipulations and does not leave a long tail on the base after the cleavage reaction.

No previously reported nucleotide analogue containing a 3′-O-alkyldithiomethyl blocking group, which is removed in a single step and which does not require an additional step to cap the resulting free SH group, has been reported for use in ion sensor SBS sequencing.

Recently, Ion Torrent, Inc., has described sequencing strategies in which the proton released as each nucleotide is incorporated into the DNA chain is captured by an ion sensor and digitized using semiconductor technology (Anderson et al. 2009; Rothberg et al. 2011). Again, however, since this output is identical no matter which of the four nucleotides is incorporated, because these strategies use natural nucleotides, this necessitates the base-by-base addition strategy, with its inherent difficulty in achieving accurate reads through homopolymeric base runs.

An SBS method has been described in which each nucleotide has a unique Raman spectroscopy peak, wherein determination of the wavenumber of the Raman peak is used to identify an incorporated nucleotide analogue (PCT International Application Publication No. WO 2012/162429, which is hereby incorporated by reference). However, using Raman spectroscopy to detect and identify nucleotide analogues suffers from low sensitivity inherent in this technique.

### SUMMARY OF THE INVENTION

The invention is directed to a method for determining the identity of a nucleotide residue of a single-stranded DNA in a solution comprising:
(a) contacting the single-stranded DNA, having a primer hybridized to a portion thereof, with a DNA polymerase and a deoxyribonucleotide triphosphate (dNTP) analogue under conditions permitting the DNA polymerase to catalyze incorporation of the dNTP analogue into the primer if it is complementary to the nucleotide residue of the single-stranded DNA which is immediately 5′ to a nucleotide residue of the single-stranded DNA hybridized to the 3′ terminal nucleotide residue of the primer, so as to form a DNA extension product, wherein (1) the dNTP analogue has the structure:

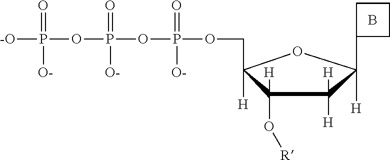

wherein B is a base and is adenine, guanine, cytosine, or thymine, and (2) R′ is (i) —CH₂N₃ or 2-nitrobenzyl, (ii) is a hydrocarbyl, or a substituted hydrocarbyl, having a mass of less than 300 daltons, or (iii) is a dithio moiety; and
(b) determining whether incorporation of the dNTP analogue into the primer to form a DNA extension product has occurred in step (a) by determining if an increase in hydrogen ion concentration of the solution has occurred, wherein (i) if the dNTP analogue has been incorporated into the primer, determining from the identity of the incorporated dNTP analogue the identity of the nucleotide residue in the single-stranded DNA complementary thereto, thereby determining the identity of the nucleotide residue in the single-stranded DNA, and (ii) if no change in hydrogen ion concentration has occurred, iteratively performing step (a), wherein in each iteration of step (a) the dNTP analogue comprises a base which is a different type of base from the type of base of the dNTP analogues in every preceding iteration of step (a), until a dNTP analogue is incorporated into the primer to form a DNA extension product, and determining from the identity of the incorporated dNTP analogue the identity of the nucleotide residue in the single-stranded DNA complementary thereto, thereby determining the identity of the nucleotide residue in the single-stranded DNA.

The invention is further directed to a method for determining the sequence of consecutive nucleotide residues in a single-stranded DNA in a solution comprising:
(a) contacting the single-stranded DNA, having a primer hybridized to a portion thereof, with a DNA polymerase and a deoxyribonucleotide triphosphate (dNTP) analogue under conditions permitting the DNA polymerase to catalyze incorporation of the dNTP analogue into the primer if it is complementary to the nucleotide residue of the single-stranded DNA which is immediately 5′ to a nucleotide residue of the single-stranded DNA hybridized to the 3′ terminal nucleotide residue of the primer, so as to form a DNA extension product, wherein (1) the dNTP analogue has the structure:

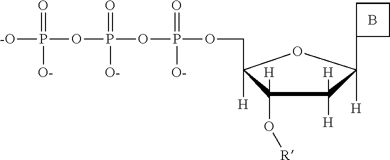

wherein B is a base and is adenine, guanine, cytosine, or thymine, and (2) R′ is (i) or —CH₂N₃, or 2-nitrobenzyl, (ii) is a hydrocarbyl, or a substituted hydrocarbyl, having a mass of less than 300 daltons, or (iii) is a dithio moiety;
(b) determining whether incorporation of the dNTP analogue has occurred in step (a) by detecting an increase in hydrogen ion concentration of the solution, wherein an increase in hydrogen ion concentration indicates that the dNTP analogue has been incorporated into the primer to form a DNA extension product, and if so, determining from the identity of the incorporated dNTP analogue the identity of the nucleotide residue in the single-stranded DNA complementary thereto, thereby determining the identity of the nucleotide residue in the single-stranded DNA, and wherein no change in hydrogen ion concentration indicates that the dNTP analogue has not been incorporated into the primer in step (a);
(c) if no change in hydrogen ion concentration has been detected in step (b), iteratively performing steps (a) and (b), wherein in each iteration of step (a) for a given nucleotide residue, the identity of which is being determined, the dNTP analogue comprises a base which is a different type of base from the type of base of the dNTP analogues in every preceding iteration of step (a) for that nucleotide residue, until a dNTP analogue is incorporated into the primer to form a DNA extension product, and determining from the identity of the incorporated dNTP analogue the identity of the nucleotide residue in the single-stranded DNA complementary thereto, thereby determining the identity of the nucleotide residue in the single-stranded DNA;
(d) if an increase in hydrogen ion concentration has been detected and a dNTP analogue is incorporated, subsequently treating the incorporated dNTP nucleotide analogue so as to replace the R′ group thereof with an H atom thereby providing a 3′ OH group at the 3′ terminal of the DNA extension product; and
(e) iteratively performing steps (a) to (d), as necessary, for each nucleotide residue of the consecutive nucleotide residues of the single-stranded DNA to be sequenced, except that in each repeat of step (a) the dNTP analogue is (i) incorporated into the DNA extension product resulting from a preceding iteration of step (a) or step (c), and (ii) complementary to a nucleotide residue of the single-stranded DNA which is immediately 5′ to a nucleotide residue of the single-stranded DNA hybridized to the 3′ terminal nucleotide residue of the DNA extension product resulting from a preceding iteration of step (a) or step (c), so as to form a subsequent DNA extension product, with the proviso that for the last nucleotide residue to be sequenced step (d) is optional,
thereby determining the identity of each of the consecutive nucleotide residues of the single-stranded DNA so as to thereby determine the sequence of the consecutive nucleotide residues of the DNA.

The invention is further directed to a method for determining the identity of a nucleotide residue of a single-stranded RNA in a solution comprising:
(a) contacting the single-stranded RNA, having an RNA primer hybridized to a portion thereof, with a polymerase and a ribonucleotide triphosphate (rNTP) analogue under conditions permitting the polymerase to catalyze incorporation of the rNTP analogue into the RNA primer if it is complementary to the nucleotide residue of the single-stranded RNA which is immediately 5′ to a nucleotide residue of the single-stranded RNA hybridized to the 3′ terminal nucleotide residue of the primer, so as to form an RNA extension product, wherein (1) the rNTP analogue has the structure:

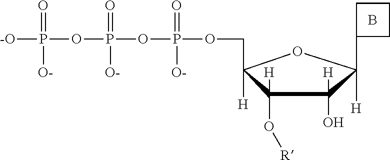

wherein B is a base and is adenine, guanine, cytosine, or uracil, and (2) R′ is (i) —CH₂N₃ or 2-nitrobenzyl, (ii) is a hydrocarbyl, or a substituted hydrocarbyl, having a mass of less than 300 daltons, or (iii) is a dithio moiety; and
(b) determining whether incorporation of the rNTP analogue into the RNA primer to form an RNA extension product has occurred in step (a) by determining if an increase in hydrogen ion concentration of the solution has occurred, wherein (i) if the rNTP analogue has been incorporated into the RNA primer, determining from the identity of the incorporated rNTP analogue the identity of the nucleotide residue in the single-stranded RNA complementary thereto, thereby determining the identity of the nucleotide residue in the single-stranded RNA, and (ii) if no change in hydrogen ion concentration has occurred, iteratively performing step (a), wherein in each iteration of step (a) the rNTP analogue comprises a base which is a different type of base from the type of base of the rNTP analogues in every preceding iteration of step (a), until an rNTP analogue is incorporated into the RNA primer to form an RNA extension product, and determining from the identity of the incorporated rNTP analogue the identity of the nucleotide residue in the single-stranded RNA complementary thereto, thereby determining the identity of the nucleotide residue in the single-stranded RNA.

The invention is further directed to a method for determining the sequence of consecutive nucleotide residues in a single-stranded RNA in a solution comprising:
(a) contacting the single-stranded RNA, having an RNA primer hybridized to a portion thereof, with a polymerase and a ribonucleotide triphosphate (rNTP) analogue under conditions permitting the polymerase to catalyze incorporation of the rNTP analogue into the RNA primer if it is complementary to the nucleotide residue of the single-stranded RNA which is immediately 5′ to a nucleotide residue of the single-stranded RNA hybridized to the 3′ terminal nucleotide residue of the RNA primer, so as to form an RNA extension product, wherein (1) the rNTP analogue has the structure:

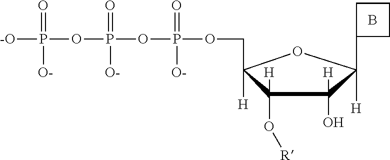

wherein B is a base and is adenine, guanine, cytosine, or uracil, and (2) R′ is (i) —CH₂N₃, or 2-nitrobenzyl, (ii) is a hydrocarbyl, or a substituted hydrocarbyl, having a mass of less than 300 daltons, or (iii) is a dithio moiety;
(b) determining whether incorporation of the rNTP analogue has occurred in step (a) by detecting an increase in hydrogen ion concentration of the solution, wherein an increase in hydrogen ion concentration indicates that the rNTP analogue has been incorporated into the RNA primer to form an RNA extension product, and if so, determining from the identity of the incorporated rNTP analogue the identity of the nucleotide residue in the single-stranded RNA complementary thereto, thereby determining the identity of the nucleotide residue in the single-stranded RNA, and wherein no change in hydrogen ion concentration indicates that the rNTP analogue has not been incorporated into the RNA primer in step (a);
(c) if no change in hydrogen ion concentration has been detected in step (b), iteratively performing steps (a) and (b), wherein in each iteration of step (a) for a given nucleotide residue, the identity of which is being determined, the rNTP analogue comprises a base which is a different type of base from the type of base of the rNTP analogues in every preceding iteration of step (a) for that nucleotide residue, until an rNTP analogue is incorporated into the primer to form an RNA extension product, and determining from the identity of the incorporated rNTP analogue the identity of the nucleotide residue in the single-stranded RNA complementary thereto, thereby determining the identity of the nucleotide residue in the single-stranded RNA;
(d) if an increase in hydrogen ion concentration has been detected and an rNTP analogue is incorporated, subsequently treating the incorporated rNTP nucleotide analogue so as to replace the R′ group thereof with an H atom thereby providing a 3′ OH group at the 3′ terminal of the RNA extension product; and
(e) iteratively performing steps (a) to (d), as necessary, for each nucleotide residue of the consecutive nucleotide residues of the single-stranded RNA to be sequenced, except that in each repeat of step (a) the rNTP analogue is (i) incorporated into the RNA extension product resulting from a preceding iteration of step (a) or step (c), and (ii) complementary to a nucleotide residue of the single-stranded RNA which is immediately 5′ to a nucleotide residue of the single-stranded RNA hybridized to the 3′ terminal nucleotide residue of the RNA extension product resulting from a preceding iteration of step (a) or step (c), so as to form a subsequent RNA extension product, with the proviso that for the last nucleotide residue to be sequenced step (d) is optional,
thereby determining the identity of each of the consecutive nucleotide residues of the single-stranded RNA so as to thereby determine the sequence of the consecutive nucleotide residues of the RNA.

The invention is further directed to a method for determining the identity of a nucleotide residue of a single-stranded RNA in a solution comprising:
(a) contacting the single-stranded RNA, having a DNA primer hybridized to a portion thereof, with a reverse transcriptase and a deoxyribonucleotide triphosphate (dNTP) analogue under conditions permitting the reverse transcriptase to catalyze incorporation of the dNTP analogue into the DNA primer if it is complementary to the nucleotide residue of the single-stranded RNA which is immediately 5′ to a nucleotide residue of the single-stranded RNA hybridized to the 3′ terminal nucleotide residue of the DNA primer, so as to form a DNA extension product, wherein (1) the dNTP analogue has the structure:

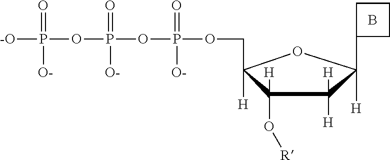

wherein B is a base and is adenine, guanine, cytosine, or thymine, and (2) R′ is (i) —CH₂N₃ or 2-nitrobenzyl, (ii) is a hydrocarbyl, or a substituted hydrocarbyl, having a mass of less than 300 daltons, or (iii) is a dithio moiety; and
(b) determining whether incorporation of the dNTP analogue into the DNA primer to form a DNA extension product has occurred in step (a) by determining if an increase in hydrogen ion concentration of the solution has occurred, wherein (i) if the dNTP analogue has been incorporated into the DNA primer, determining from the identity of the incorporated dNTP analogue the identity of the nucleotide residue in the single-stranded RNA complementary thereto, thereby determining the identity of the nucleotide residue in the single-stranded RNA, and (ii) if no change in hydrogen ion concentration has occurred, iteratively performing step (a), wherein in each iteration of step (a) the dNTP analogue comprises a base which is a different type of base from the type of base of the dNTP analogues in every preceding iteration of step (a), until a dNTP analogue is incorporated into the DNA primer to form a DNA extension product, and determining from the identity of the incorporated dNTP analogue the identity of the nucleotide residue in the single-stranded DNA complementary thereto, thereby determining the identity of the nucleotide residue in the single-stranded DNA.

The invention is further directed to a method for determining the sequence of consecutive nucleotide residues in a single-stranded RNA in a solution comprising:
(a) contacting the single-stranded RNA, having a DNA primer hybridized to a portion thereof, with a reverse transcriptase and a deoxyribonucleotide triphosphate (dNTP) analogue under conditions permitting the reverse transcriptase to catalyze incorporation of the dNTP analogue into the primer if it is complementary to the nucleotide residue of the single-stranded RNA which is immediately 5′ to a nucleotide residue of the single-stranded RNA hybridized to the 3′ terminal nucleotide residue of the DNA primer, so as to form a DNA extension product, wherein (1) the dNTP analogue has the structure:

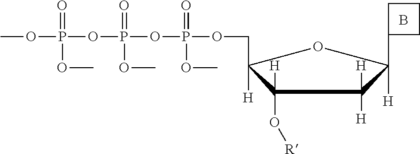

wherein B is a base and is adenine, guanine, cytosine, or thymine, and (2) R′ is (i) —CH₂N₃ or 2-nitrobenzyl, (ii) is a hydrocarbyl, or a substituted hydrocarbyl, having a mass of less than 300 daltons, or (iii) is a dithio moiety;
(b) determining whether incorporation of the dNTP analogue has occurred in step (a) by detecting an increase in hydrogen ion concentration of the solution, wherein an increase in hydrogen ion concentration indicates that the dNTP analogue has been incorporated into the DNA primer to form an RNA extension product, and if so, determining from the identity of the incorporated dNTP analogue the identity of the nucleotide residue in the single-stranded RNA complementary thereto, thereby determining the identity of the nucleotide residue in the single-stranded RNA, and wherein no change in hydrogen ion concentration indicates that the dNTP analogue has not been incorporated into the DNA primer in step (a);
(c) if no change in hydrogen ion concentration has been detected in step (b), iteratively performing steps (a) and (b), wherein in each iteration of step (a) for a given nucleotide residue, the identity of which is being determined, the dNTP analogue comprises a base which is a different type of base from the type of base of the dNTP analogues in every preceding iteration of step (a) for that nucleotide residue, until a dNTP analogue is incorporated into the DNA primer to form a DNA extension product, and determining from the identity of the incorporated dNTP analogue the identity of the nucleotide residue in the single-stranded RNA complementary thereto, thereby determining the identity of the nucleotide residue in the single-stranded RNA;
(d) if an increase in hydrogen ion concentration has been detected and a dNTP analogue is incorporated, subsequently treating the incorporated dNTP nucleotide analogue so as to replace the R′ group thereof with an H atom thereby providing a 3′ OH group at the 3′ terminal of the DNA extension product; and
(e) iteratively performing steps (a) to (d), as necessary, for each nucleotide residue of the consecutive nucleotide residues of the single-stranded RNA to be sequenced, except that in each repeat of step (a) the dNTP analogue is (i) incorporated into the DNA extension product resulting from a preceding iteration of step (a) or step (c), and (ii) complementary to a nucleotide residue of the single-stranded RNA which is immediately 5′ to a nucleotide residue of the single-stranded RNA hybridized to the 3′ terminal nucleotide residue of the DNA extension product resulting from a preceding iteration of step (a) or step (c), so as to form a subsequent DNA extension product, with the proviso that for the last nucleotide residue to be sequenced step (d) is optional,
thereby determining the identity of each of the consecutive nucleotide residues of the single-stranded RNA so as to thereby determine the sequence of the consecutive nucleotide residues of the RNA.

The invention provides a nucleotide analogue comprising (i) a base, (ii) a deoxyribose or ribose, and (iii) a dithio moiety bound to the 3′-oxygen of the deoxyribose or ribose.

The invention also provides a process for producing a 3′-O-ethyldithiomethyl nucleoside, comprising:
a) providing,
   1) a nucleoside,
   2) acetic acid,
   3) acetic anhydride, and
   4) DMSO
under conditions permitting the production of a 3′-O-methylthiomethyl nucleoside;
b) contacting the 3′-O-methylthiomethyl nucleoside produced in part a) with trimethylamine, molecular sieve, and sulfuryl chloride under conditions permitting the production of a 3′-O-chloromethyl nucleoside;
c) contacting the 3′-O-chloromethyl nucleoside produced in part b) with potassium p-toluenethiosulfonate and ethanethiol under conditions permitting the production of a 3′-O-ethyldithiomethyl nucleoside.

### BRIEF DESCRIPTION OF THE FIGURES

FIG. 1. NRTs with various blocking groups (R) at the 3′-OH position. Photo-cleavage of 2-nitrobenzyl group (lower center) or chemical cleavage of allyl (lower left) azidomethyl groups (lower right), and dithiomethyl (bottom) restores the 3′-OH for subsequent reaction cycles.

FIG. 2. Comparison of reversible terminator-pyrosequencing of DNA using 3′-O-(2-nitrobenzyl)-dNTPs with conventional pyrosequencing using natural nucleotides (NB=2-nitrobenzyl). (A) The self-priming DNA template with stretches of homopolymeric regions (5 C's, 5 T's, 3 A's, 2 C's, 2 G's, 2 T's and 2 C's) was sequenced using 3′-O-(2-nitrobenzyl)-dNTPs. The homopolymeric regions are clearly identified with each peak corresponding to the identity of each base in the DNA template. (B) Pyrosequencing data using natural nucleotides. The homopolymeric regions produced two large peaks corresponding to the stretches of G's and A's and 5 smaller peaks corresponding to stretches of T's, G's, C's, A's and G's. However, it is very difficult to decipher the exact sequence from the data.

FIG. 3. Ion Sensor Sequencing By Synthesis (SBS) with NRTs. Surface-attached templates are extended with NRTs, added one at a time. If there is incorporation, a H+ ion is released and detected. After cleavage of the blocking group, the next cycle is initiated. Because the NRTs force the reactions to pause after each cycle, the lengths of homopolymers are determined with precision.

FIG. 4. Mechanism of cleavage of S—S bridge and generation of nucleotide free of —SH group.

FIG. 5. Structures of four 3′-O-alkyldithiomethyl-dNTPs (3′-O-DTM-dNTPs).

FIG. 6. Chemical structures of the four 3′-O-Et-dithiomethyl-dNTPs (3′-O-DTM-dNTPs or 3′-O-Et-SS-dNTPs), nucleotide reversible terminators: 3′-O-Et-SS-dATP, 3′-O-Et-SS-dGTP, 3′-O-Et-SS-dCTP, and 3′-O-Et-SS-dTTP.

FIG. 7. Scheme for synthesis of 3′-O-ethyldithiomethyl-dTTP (7a).

FIG. 8. Scheme for synthesis of 3′-O-ethyldithiomethyl-dGTP (9b).

FIG. 9. Scheme for synthesis of 3′-O-ethyldithiomethyl-dATP (8c).

FIG. 10. Scheme for synthesis of 3′-O-ethyldithiomethyl-dCTP (7d).

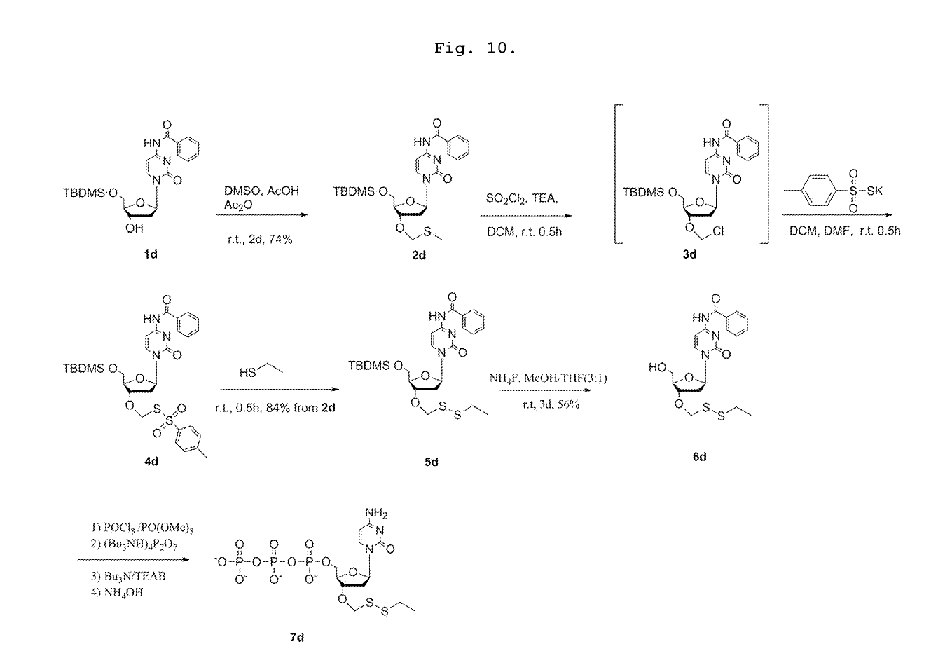

FIG. 11. Scheme of continuous DNA sequencing by synthesis (left) using four 3′-O-Et-dithiomethyl-dNTPs reversible terminators (3′-O-SS-Et-dNTPs or 3′-O-DTM-dNTPs) (Structures in FIG. 6) and MALDI-TOF MS spectra (right) obtained from each step of extension and cleavage. THP=(tris(hydroxypropyl)phosphine). The masses of the expected extension products are 4381, 4670, 4995, and 5295 Da respectively. The masses of the expected cleavage products are 4272, 4561, 4888, and 5186 Da. The measured masses shown (right) are within the resolution of MALDI-TOF MS.

FIG. 12. Structures of four 3′-O-t-butyl-SS-dNTPs (3′-O-DTM-dNTPs).

FIG. 13. Scheme of continuous DNA sequencing by synthesis (left) using four 3′-O-t-Bu-SS-dNTPs reversible terminators (Structures in FIG. 12) and MALDI-TOF MS spectra Fig.D) obtained from each step of extension and cleavage. The masses of the expected extension products are 4404, 4697, 5024, and 5328 Daltons respectively. The measured masses shown (right) of the expected cleavage products are 4272, 4563, 4888, and 5199 Daltons.

FIG. 14. Demonstration of walking strategy. The DNA template and primer shown above were used (the portion of the template shown in green is the primer binding region) and incubation was carried out using Therminator IX DNA polymerase, dATP, dCTP, dTTP and 3′-O-t-butyl-SS-dGTP. After the first walk, the primer was extended to the point of the next C in the template (rightmost C highlighted in red in the template strand). The size of the extension product was 5330 Daltons (5328 Da expected) as shown in the top left MALDI-TOF MS trace. After cleavage with THP, the 5198 Da product shown at the top right was observed (5194 Da expected). A second walk was performed with Therminator IX DNA polymerase, dATP, dCTP, dTTP and 3′-O-t-butyl-SS-dGTP to obtain the product shown in the middle left trace (7771 Da observed, 7775 Da expected to reach the middle C highlighted in red). After cleavage, a product of 7643 Da was obtained (expected 7641 Da). Finally a third walk and cleavage were performed, giving products of 9625 Da (9628 Da expected for the leftmost red highlighted C) and 9513 Da (9493 Da expected), respectively. This demonstrates the ability to use the 3′-O-t-butyl-SS-nucleotide as a terminator for walking reactions. These can be incorporated into a combined sequencing/walking scheme.

FIG. 15. General structures of 3′-O-DTM-dNTPs.

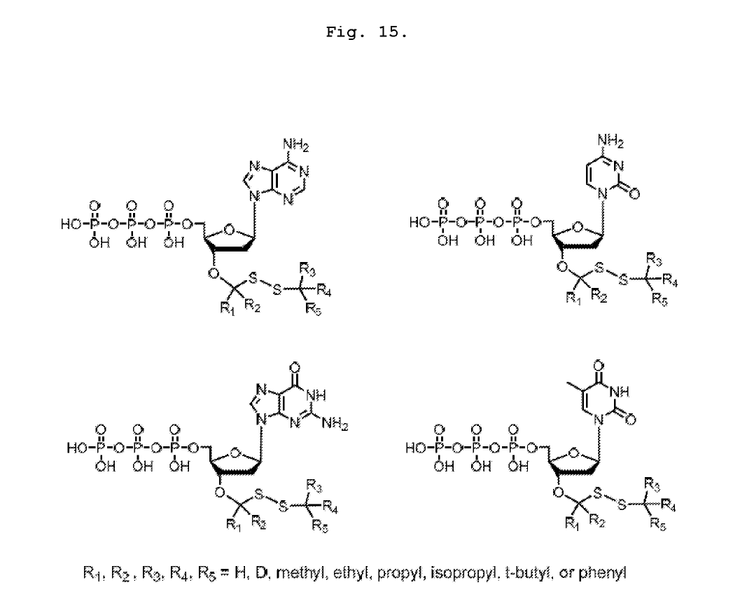

FIG. 16. 3′-O-DTM-dNTPs with various blocking group modifications, which can be used for the methods disclosed herein.

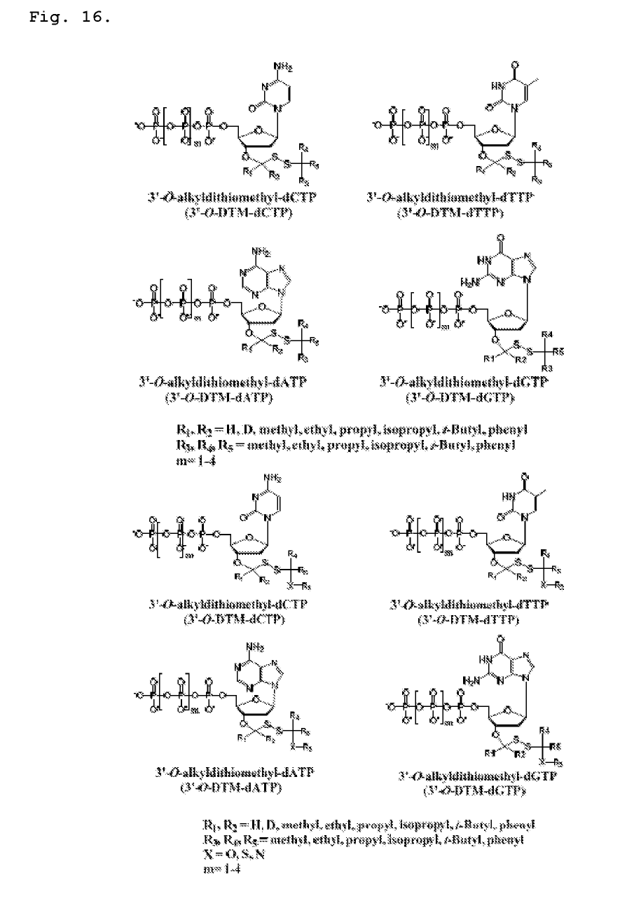

FIG. 17. Synthesis of 3′-O-t-butyl-SS-dTTP (5a).

FIG. 18. Synthesis of 3′-O-t-butyl-SS-dGTP (G5).

FIG. 19. Synthesis of 3′-O-t-butyl-SS-dATP (A5).

FIG. 20. Synthesis of 3′-O-t-butyl-SS-dCTP (C5).

### DETAILED DESCRIPTION OF THE INVENTION

The present invention is directed to a method for determining the identity of a nucleotide residue of a single-stranded DNA in a solution comprising:
(a) contacting the single-stranded DNA, having a primer hybridized to a portion thereof, with a DNA polymerase and a deoxyribonucleotide triphosphate (dNTP) analogue under conditions permitting the DNA polymerase to catalyze incorporation of the dNTP analogue into the primer if it is complementary to the nucleotide residue of the single-stranded DNA which is immediately 5′ to a nucleotide residue of the single-stranded DNA hybridized to the 3′ terminal nucleotide residue of the primer, so as to form a DNA extension product, wherein (1) the dNTP analogue has the structure:

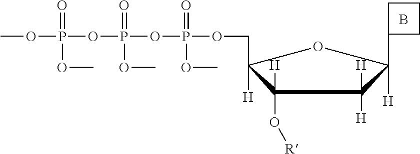

wherein B is a base and is adenine, guanine, cytosine, or thymine, and (2) R′ is (i) —CH₂N₃ or 2-nitrobenzyl, (ii) is a hydrocarbyl, or a substituted hydrocarbyl, having a mass of less than 300 daltons, or (iii) is a dithio moiety; and
(b) determining whether incorporation of the dNTP analogue into the primer to form a DNA extension product has occurred in step (a) by determining if an increase in hydrogen ion concentration of the solution has occurred, wherein (i) if the dNTP analogue has been incorporated into the primer, determining from the identity of the incorporated dNTP analogue the identity of the nucleotide residue in the single-stranded DNA complementary thereto, thereby determining the identity of the nucleotide residue in the single-stranded DNA, and (ii) if no change in hydrogen ion concentration has occurred, iteratively performing step (a), wherein in each iteration of step (a) the dNTP analogue comprises a base which is a different type of base from the type of base of the dNTP analogues in every preceding iteration of step (a), until a dNTP analogue is incorporated into the primer to form a DNA extension product, and determining from the identity of the incorporated dNTP analogue the identity of the nucleotide residue in the single-stranded DNA complementary thereto, thereby determining the identity of the nucleotide residue in the single-stranded DNA.

The present invention is further directed to a method for determining the sequence of consecutive nucleotide residues in a single-stranded DNA in a solution comprising:
(a) contacting the single-stranded DNA, having a primer hybridized to a portion thereof, with a DNA polymerase and a deoxyribonucleotide triphosphate (dNTP) analogue under conditions permitting the DNA polymerase to catalyze incorporation of the dNTP analogue into the primer if it is complementary to the nucleotide residue of the single-stranded DNA which is immediately 5′ to a nucleotide residue of the single-stranded DNA hybridized to the 3′ terminal nucleotide residue of the primer, so as to form a DNA extension product, wherein (1) the dNTP analogue has the structure:

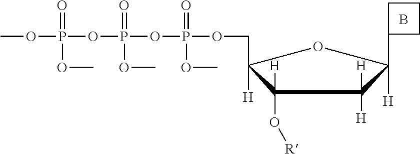

wherein B is a base and is adenine, guanine, cytosine, or thymine, and (2) R′ is (i) —CH₂N₃, or 2-nitrobenzyl, (ii) is a hydrocarbyl, or a substituted hydrocarbyl, having a mass of less than 300 daltons, or (iii) is a dithio moiety;
(b) determining whether incorporation of the dNTP analogue has occurred in step (a) by detecting an increase in hydrogen ion concentration of the solution, wherein an increase in hydrogen ion concentration indicates that the dNTP analogue has been incorporated into the primer to form a DNA extension product, and if so, determining from the identity of the incorporated dNTP analogue the identity of the nucleotide residue in the single-stranded DNA complementary thereto, thereby determining the identity of the nucleotide residue in the single-stranded DNA, and wherein no change in hydrogen ion concentration indicates that the dNTP analogue has not been incorporated into the primer in step (a);
(c) if no change in hydrogen ion concentration has been detected in step (b), iteratively performing steps (a) and (b), wherein in each iteration of step (a) for a given nucleotide residue, the identity of which is being determined, the dNTP analogue comprises a base which is a different type of base from the type of base of the dNTP analogues in every preceding iteration of step (a) for that nucleotide residue, until a dNTP analogue is incorporated into the primer to form a DNA extension product, and determining from the identity of the incorporated dNTP analogue the identity of the nucleotide residue in the single-stranded DNA complementary thereto, thereby determining the identity of the nucleotide residue in the single-stranded DNA;
(d) if an increase in hydrogen ion concentration has been detected and a dNTP analogue is incorporated, subsequently treating the incorporated dNTP nucleotide analogue so as to replace the R′ group thereof with an H atom thereby providing a 3′ OH group at the 3′ terminal of the DNA extension product; and
(e) iteratively performing steps (a) to (d), as necessary, for each nucleotide residue of the consecutive nucleotide residues of the single-stranded DNA to be sequenced, except that in each repeat of step (a) the dNTP analogue is (i) incorporated into the DNA extension product resulting from a preceding iteration of step (a) or step (c), and (ii) complementary to a nucleotide residue of the single-stranded DNA which is immediately 5′ to a nucleotide residue of the single-stranded DNA hybridized to the 3′ terminal nucleotide residue of the DNA extension product resulting from a preceding iteration of step (a) or step (c), so as to form a subsequent DNA extension product, with the proviso that for the last nucleotide residue to be sequenced step (d) is optional,
thereby determining the identity of each of the consecutive nucleotide residues of the single-stranded DNA so as to thereby determine the sequence of the consecutive nucleotide residues of the DNA.

The invention is further directed to a method for determining the identity of a nucleotide residue of a single-stranded RNA in a solution comprising:
(a) contacting the single-stranded RNA, having an RNA primer hybridized to a portion thereof, with a polymerase and a ribonucleotide triphosphate (rNTP) analogue under conditions permitting the polymerase to catalyze incorporation of the rNTP analogue into the RNA primer if it is complementary to the nucleotide residue of the single-stranded RNA which is immediately 5′ to a nucleotide residue of the single-stranded RNA hybridized to the 3′ terminal nucleotide residue of the primer, so as to form an RNA extension product, wherein (1) the rNTP analogue has the structure:

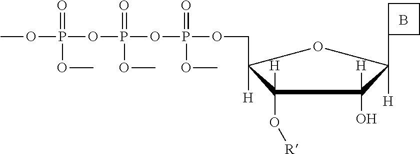

wherein B is a base and is adenine, guanine, cytosine, or uracil, and (2) R′ is (i) —CH₂N₃ or 2-nitrobenzyl, (ii) is a hydrocarbyl, or a substituted hydrocarbyl, having a mass of less than 300 daltons, or (iii) is a dithio moiety; and
(b) determining whether incorporation of the rNTP analogue into the RNA primer to form an RNA extension product has occurred in step (a) by determining if an increase in hydrogen ion concentration of the solution has occurred, wherein (i) if the rNTP analogue has been incorporated into the RNA primer, determining from the identity of the incorporated rNTP analogue the identity of the nucleotide residue in the single-stranded RNA complementary thereto, thereby determining the identity of the nucleotide residue in the single-stranded RNA, and (ii) if no change in hydrogen ion concentration has occurred, iteratively performing step (a), wherein in each iteration of step (a) the rNTP analogue comprises a base which is a different type of base from the type of base of the rNTP analogues in every preceding iteration of step (a), until an rNTP analogue is incorporated into the RNA primer to form an RNA extension product, and determining from the identity of the incorporated rNTP analogue the identity of the nucleotide residue in the single-stranded RNA complementary thereto, thereby determining the identity of the nucleotide residue in the single-stranded RNA.

The invention is further directed to a method for determining the sequence of consecutive nucleotide residues in a single-stranded RNA in a solution comprising:
(a) contacting the single-stranded RNA, having an RNA primer hybridized to a portion thereof, with a polymerase and a ribonucleotide triphosphate (rNTP) analogue under conditions permitting the polymerase to catalyze incorporation of the rNTP analogue into the RNA primer if it is complementary to the nucleotide residue of the single-stranded RNA which is immediately 5′ to a nucleotide residue of the single-stranded RNA hybridized to the 3′ terminal nucleotide residue of the RNA primer, so as to form an RNA extension product, wherein (1) the rNTP analogue has the structure:

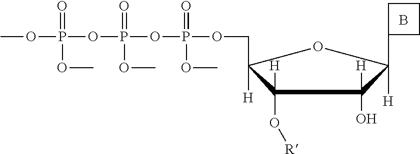

wherein B is a base and is adenine, guanine, cytosine, or uracil, and (2) R′ is (i) —CH₂N₃, or 2-nitrobenzyl, (ii) is a hydrocarbyl, or a substituted hydrocarbyl, having a mass of less than 300 daltons, or (iii) is a dithio moiety;
(b) determining whether incorporation of the rNTP analogue has occurred in step (a) by detecting an increase in hydrogen ion concentration of the solution, wherein an increase in hydrogen ion concentration indicates that the rNTP analogue has been incorporated into the RNA primer to form an RNA extension product, and if so, determining from the identity of the incorporated rNTP analogue the identity of the nucleotide residue in the single-stranded RNA complementary thereto, thereby determining the identity of the nucleotide residue in the single-stranded RNA, and wherein no change in hydrogen ion concentration indicates that the rNTP analogue has not been incorporated into the RNA primer in step (a);
(c) if no change in hydrogen ion concentration has been detected in step (b), iteratively performing steps (a) and (b), wherein in each iteration of step (a) for a given nucleotide residue, the identity of which is being determined, the rNTP analogue comprises a base which is a different type of base from the type of base of the rNTP analogues in every preceding iteration of step (a) for that nucleotide residue, until an rNTP analogue is incorporated into the primer to form an RNA extension product, and determining from the identity of the incorporated rNTP analogue the identity of the nucleotide residue in the single-stranded RNA complementary thereto, thereby determining the identity of the nucleotide residue in the single-stranded RNA;
(d) if an increase in hydrogen ion concentration has been detected and an rNTP analogue is incorporated, subsequently treating the incorporated rNTP nucleotide analogue so as to replace the R′ group thereof with an H atom thereby providing a 3′ OH group at the 3′ terminal of the RNA extension product; and
(e) iteratively performing steps (a) to (d), as necessary, for each nucleotide residue of the consecutive nucleotide residues of the single-stranded RNA to be sequenced, except that in each repeat of step (a) the rNTP analogue is (i) incorporated into the RNA extension product resulting from a preceding iteration of step (a) or step (c), and (ii) complementary to a nucleotide residue of the single-stranded RNA which is immediately 5′ to a nucleotide residue of the single-stranded RNA hybridized to the 3′ terminal nucleotide residue of the RNA extension product resulting from a preceding iteration of step (a) or step (c), so as to form a subsequent RNA extension product, with the proviso that for the last nucleotide residue to be sequenced step (d) is optional,
thereby determining the identity of each of the consecutive nucleotide residues of the single-stranded RNA so as to thereby determine the sequence of the consecutive nucleotide residues of the RNA.

The invention is further directed to a method for determining the identity of a nucleotide residue of a single-stranded RNA in a solution comprising:
(a) contacting the single-stranded RNA, having a DNA primer hybridized to a portion thereof, with a reverse transcriptase and a deoxyribonucleotide triphosphate (dNTP) analogue under conditions permitting the reverse transcriptase to catalyze incorporation of the dNTP analogue into the DNA primer if it is complementary to the nucleotide residue of the single-stranded RNA which is immediately 5′ to a nucleotide residue of the single-stranded RNA hybridized to the 3′ terminal nucleotide residue of the DNA primer, so as to form a DNA extension product, wherein (1) the dNTP analogue has the structure:

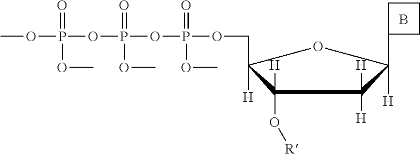

wherein B is a base and is adenine, guanine, cytosine, or thymine, and (2) R′ is (i) —CH₂N₃ or 2-nitrobenzyl, (ii) is a hydrocarbyl, or a substituted hydrocarbyl, having a mass of less than 300 daltons, or (iii) is a dithio moiety; and
(b) determining whether incorporation of the dNTP analogue into the DNA primer to form a DNA extension product has occurred in step (a) by determining if an increase in hydrogen ion concentration of the solution has occurred, wherein (i) if the dNTP analogue has been incorporated into the DNA primer, determining from the identity of the incorporated dNTP analogue the identity of the nucleotide residue in the single-stranded RNA complementary thereto, thereby determining the identity of the nucleotide residue in the single-stranded RNA, and (ii) if no change in hydrogen ion concentration has occurred, iteratively performing step (a), wherein in each iteration of step (a) the dNTP analogue comprises a base which is a different type of base from the type of base of the dNTP analogues in every preceding iteration of step (a), until a dNTP analogue is incorporated into the DNA primer to form a DNA extension product, and determining from the identity of the incorporated dNTP analogue the identity of the nucleotide residue in the single-stranded RNA complementary thereto, thereby determining the identity of the nucleotide residue in the single-stranded RNA.

The invention is further directed to a method for determining the sequence of consecutive nucleotide residues in a single-stranded RNA in a solution comprising:
(a) contacting the single-stranded RNA, having a DNA primer hybridized to a portion thereof, with a reverse transcriptase and a deoxyribonucleotide triphosphate (dNTP) analogue under conditions permitting the reverse transcriptase to catalyze incorporation of the dNTP analogue into the primer if it is complementary to the nucleotide residue of the single-stranded RNA which is immediately 5′ to a nucleotide residue of the single-stranded RNA hybridized to the 3′ terminal nucleotide residue of the DNA primer, so as to form a DNA extension product, wherein (1) the dNTP analogue has the structure:

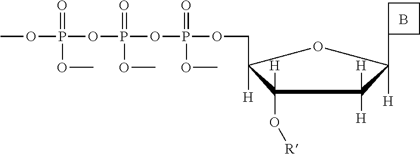

wherein B is a base and is adenine, guanine, cytosine, or thymine, and (2) R′ is (i) —CH₂N₃ or 2-nitrobenzyl, (ii) is a hydrocarbyl, or a substituted hydrocarbyl, having a mass of less than 300 daltons, or (iii) is a dithio moiety;
(b) determining whether incorporation of the dNTP analogue has occurred in step (a) by detecting an increase in hydrogen ion concentration of the solution, wherein an increase in hydrogen ion concentration indicates that the dNTP analogue has been incorporated into the DNA primer to form a DNA extension product, and if so, determining from the identity of the incorporated dNTP analogue the identity of the nucleotide residue in the single-stranded RNA complementary thereto, thereby determining the identity of the nucleotide residue in the single-stranded RNA, and wherein no change in hydrogen ion concentration indicates that the dNTP analogue has not been incorporated into the DNA primer in step (a);
(c) if no change in hydrogen ion concentration has been detected in step (b), iteratively performing steps (a) and (b), wherein in each iteration of step (a) for a given nucleotide residue, the identity of which is being determined, the dNTP analogue comprises a base which is a different type of base from the type of base of the dNTP analogues in every preceding iteration of step (a) for that nucleotide residue, until a dNTP analogue is incorporated into the DNA primer to form a DNA extension product, and determining from the identity of the incorporated dNTP analogue the identity of the nucleotide residue in the single-stranded RNA complementary thereto, thereby determining the identity of the nucleotide residue in the single-stranded RNA;
(d) if an increase in hydrogen ion concentration has been detected and a dNTP analogue is incorporated, subsequently treating the incorporated dNTP nucleotide analogue so as to replace the R′ group thereof with an H atom thereby providing a 3′ OH group at the 3′ terminal of the DNA extension product; and
(e) iteratively performing steps (a) to (d), as necessary, for each nucleotide residue of the consecutive nucleotide residues of the single-stranded RNA to be sequenced, except that in each repeat of step (a) the dNTP analogue is (i) incorporated into the DNA extension product resulting from a preceding iteration of step (a) or step (c), and (ii) complementary to a nucleotide residue of the single-stranded RNA which is immediately 5′ to a nucleotide residue of the single-stranded RNA hybridized to the 3′ terminal nucleotide residue of the DNA extension product resulting from a preceding iteration of step (a) or step (c), so as to form a subsequent DNA extension product, with the proviso that for the last nucleotide residue to be sequenced step (d) is optional,
thereby determining the identity of each of the consecutive nucleotide residues of the single-stranded RNA so as to thereby determine the sequence of the consecutive nucleotide residues of the RNA.

In one embodiment of any of the inventions described herein, R′ is —CH₂N₃.

In another embodiment of any of the inventions described herein, R′ is a substituted hydrocarbyl, and is a nitrobenzyl. In a further embodiment, R′ is a 2-nitrobenzyl.

In a further embodiment of any of the inventions described herein, R′ has the structure:

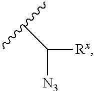

where R^{x} is, independently, a C₁-C₅ alkyl, a C₂-C₅ alkenyl, or a C₂-C₅ alkynyl, which is substituted or unsubstituted and which has a mass of less than 300 daltons.

In another embodiment, R′ has the structure:

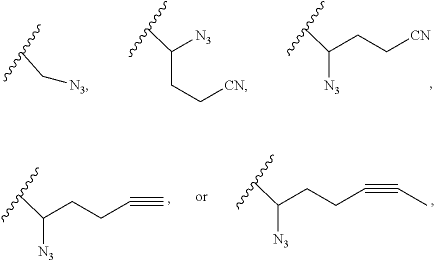

wherein the wavy line indicates the point of attachment to the 3′ oxygen atom.

In another embodiment of any of the inventions described herein, R′ is a hydrocarbyl, and is allyl (—CH₂—CH═CH₂).

In another embodiment of any of the inventions described herein, R′ is a dithio moiety.

In another embodiment of any of the inventions described herein, R′ is an alkyldithiomethyl moiety. In a further embodiment, each alkyldithiomethyl moiety has the structure:

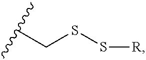

wherein R is the alkyl portion of the alkyldithiomethyl moiety and the wavy line represents the point of connection to the 3′-oxygen. In yet a further embodiment, R′ is an alkyldithiomethyl independently selected from the group consisting of methyldithiomethyl, ethyldithiomethyl, propyldithiomethyl, isopropyldithiomethyl, butyldithiomethyl, t-butyldithiomethyl, and phenyldithiomethyl. In a further embodiment, the alkyldithiomethyl moiety is a t-butyldithiomethyl moiety.

### Dithio Moiety

As used herein, and in all embodiments of the inventions disclosed, unless otherwise indicated, a deoxyribonucleotide triphosphate (dNTP) analogue or a ribonucleotide triphosphate (rNTP) analogue having an R′ which is a dithio moiety is an analogue having the structure:

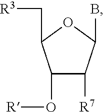

wherein, B is a base. R⁷ is H or OH. R³ is —OH, monophosphate, diphosphate, triphosphate, polyphosphate or a nucleic acid.

In some embodiments, R′ has the structure:

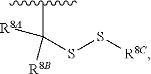

wherein each of R^{8A} R^{8B} is independently hydrogen, CH₃, —CX₃, —CHX₂, —CH₂X, —OCX₃, —OCH₂X, —OCHX₂, —CN, —OH, —SH, —NH₂, substituted (e.g., substituted with a substituent group, size-limited substituent group, or lower substituent group) or unsubstituted alkyl, substituted (e.g., substituted with a substituent group, size-limited substituent group, or lower substituent group) or unsubstituted heteroalkyl, substituted (e.g., substituted with a substituent group, size-limited substituent group, or lower substituent group) or unsubstituted cycloalkyl, substituted (e.g., substituted with a substituent group, size-limited substituent group, or lower substituent group) or unsubstituted heterocycloalkyl, substituted (e.g., substituted with a substituent group, size-limited substituent group, or lower substituent group) or unsubstituted aryl, or substituted (e.g., substituted with a substituent group, size-limited substituent group, or lower substituent group) or unsubstituted heteroaryl. R^{8C} is hydrogen, CH₃, —CX₃, —CHX₂, —CH₂X, —OCX₃, —OCH₂X, —OCHX₂, —CN, —OH, —SH, —NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{8C} is independently unsubstituted phenyl. In further embodiments, each of R^{8A} and R^{8B} is independently hydrogen, CH₃, —CX₃, —CHX₂, —CH₂X, —OCX₃, —OCH₂X, —OCHX₂, —CN, —OH, —SH, —NH₂, substituted (e.g., substituted with a substituent group, size-limited substituent group, or lower substituent group) or unsubstituted C₁-C₆ alkyl, substituted (e.g., substituted with a substituent group, size-limited substituent group, or lower substituent group) or unsubstituted 2 to 6 membered heteroalkyl, substituted (e.g., substituted with a substituent group, size-limited substituent group, or lower substituent group) or unsubstituted C₃-C₆ cycloalkyl, substituted (e.g., substituted with a substituent group, size-limited substituent group, or lower substituent group) or unsubstituted 3 to 6 membered heterocycloalkyl, substituted (e.g., substituted with a substituent group, size-limited substituent group, or lower substituent group) or unsubstituted phenyl, or substituted (e.g., substituted with a substituent group, size-limited substituent group, or lower substituent group) or unsubstituted 5 to 6 membered heteroaryl. The symbol X is independently halogen.

In further embodiments, R′ has the structure:

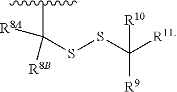

Wherein, R^{8A}, R^{8B}, R⁹, R¹⁰, and R¹¹ are each independently hydrogen, CH₃, —CX₃, —CHX₂, —CH₂X, —OCX₃, —OCH₂X, —OCHX₂, —CN, —OH, —SH, —NH₂, substituted (e.g., substituted with a substituent group, size-limited substituent group, or lower substituent group) or unsubstituted alkyl, substituted (e.g., substituted with a substituent group, size-limited substituent group, or lower substituent group) or unsubstituted heteroalkyl, substituted (e.g., substituted with a substituent group, size-limited substituent group, or lower substituent group) or unsubstituted cycloalkyl, substituted (e.g., substituted with a substituent group, size-limited substituent group, or lower substituent group) or unsubstituted heterocycloalkyl, substituted (e.g., substituted with a substituent group, size-limited substituent group, or lower substituent group) or unsubstituted aryl, or substituted (e.g., substituted with a substituent group, size-limited substituent group, or lower substituent group) or unsubstituted heteroaryl. The symbol X is independently halogen.

In further embodiments, R^{8A}, R^{8B}, R⁹, R¹⁰, and R¹¹ are each independently hydrogen, CH₃, —CX₃, —CHX₂, —CH₂X, —OCX₃, —OCH₂X, —OCHX₂, —CN, —OH, —SH, —NH₂, substituted (e.g., substituted with a substituent group, size-limited substituent group, or lower substituent group) or unsubstituted C₁-C₆ alkyl, substituted (e.g., substituted with a substituent group, size-limited substituent group, or lower substituent group) or unsubstituted 2 to 6 membered heteroalkyl, substituted (e.g., substituted with a substituent group, size-limited substituent group, or lower substituent group) or unsubstituted C₃-C₆ cycloalkyl, substituted (e.g., substituted with a substituent group, size-limited substituent group, or lower substituent group) or unsubstituted 3 to 6 membered heterocycloalkyl, substituted (e.g., substituted with a substituent group, size-limited substituent group, or lower substituent group) or unsubstituted phenyl, or substituted (e.g., substituted with a substituent group, size-limited substituent group, or lower substituent group) or unsubstituted 5 to 6 membered heteroaryl. The symbol X is independently halogen.

In further embodiments, R⁹, R¹⁰, and R¹¹ are independently unsubstituted alkyl or unsubstituted heteroalkyl. In embodiments, R⁹, R¹⁰, and R¹¹ are independently unsubstituted C₁-C₆ alkyl or unsubstituted 2 to 4 membered heteroalkyl. In embodiments, R⁹, R¹⁰, and R¹¹ are independently unsubstituted C₁-C₆ alkyl or unsubstituted 2 to 4 membered heteroalkyl. In embodiments, R⁹, R¹⁰, and R¹¹ are independently unsubstituted methyl or unsubstituted methoxy. In embodiments, R^{8A}, R^{8B}, R⁹, R¹⁰, and R¹¹ are independently hydrogen or unsubstituted methyl.

In embodiments, R^{8A} and R^{8B} are hydrogen and R⁹, R¹⁰, and R¹¹ are unsubstituted methyl.

In further embodiments, R^{8A}, R^{8B}, R⁹, R¹⁰, and R¹¹ are each independently hydrogen, deuterium, —C(CH₃)₃, —CH(CH₃)₂, —CH₂CH₂CH₃, —CH₂CH₃, —CH₃, OC(CH₃)₃, —OCH(CH₃)₂, —OCH₂CH₂CH₃, —OCH₂CH₃, —OCH₃, —SC(CH₃)₃, —SCH(CH₃)₂, —SCH₂CH₂CH₃, —SCH₂CH₃, —SCH₃, —NHC(CH₃)₃, —NHCH(CH₃)₂, —NHCH₂CH₂CH₃, —NHCH₂CH₃, —NHCH₃, —CN, or -Ph.

In further embodiments, R^{8A}, R^{8B}, R⁹, R¹⁰, and R¹¹ are each independently hydrogen, —CH₃, —CX₃, —CHX₂, —CH₂X, —CN, —Ph. The symbol X is independently halogen.

In further embodiments, R8^{A} and R8^{B} are hydrogen, and R′ has the structure:

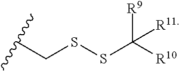

In further embodiments, R^{8A} and R^{8B} are independently hydrogen or unsubstituted alkyl; R⁹, R¹⁰, and R¹¹ are independently unsubstituted alkyl or unsubstituted heteroalkyl. In further embodiments, R^{8A} and R^{8B} are independently hydrogen or unsubstituted C₁-C₄ alkyl; and R⁹, R¹⁰, and R¹¹ are independently unsubstituted C₁-C₆ alkyl or unsubstituted 2 to 4 membered heteroalkyl.

In further embodiments, R^{8A} and R^{8B} are independently hydrogen; and R⁹, R¹⁰, and R¹¹ are independently unsubstituted C₁-C₆ alkyl or unsubstituted 2 to 4 membered heteroalkyl.

In further embodiments, R^{8A} and R^{8B} are independently hydrogen; and R⁹, R¹⁰, and R¹¹ are independently unsubstituted methyl or unsubstituted methoxy.

In further embodiments, R′ has the structure:

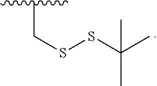 [SMILES: *CSSC(C)(C)C]

In embodiments, B is a divalent cytosine or a derivative thereof, divalent guanine or a derivative thereof, divalent adenine or a derivative thereof, divalent thymine or a derivative thereof, divalent uracil or a derivative thereof, divalent hypoxanthine or a derivative thereof, divalent xanthine or a derivative thereof, deaza-adenine or a derivative thereof, deaza-guanine or a derivative thereof, deaza-hypoxanthine or a derivative thereof divalent 7-methylguanine or a derivative thereof, divalent 5,6-dihydrouracil or a derivative thereof, divalent 5-methylcytosine or a derivative thereof, or divalent 5-hydroxymethylcytosine or a derivative thereof.

In embodiments, B is a divalent cytosine, divalent guanine, divalent adenine, divalent thymine, divalent uracil, divalent hypoxanthine, divalent xanthine, deaza-adenine, deaza-guanine, deaza-hypoxanthine or a derivative thereof divalent 7-methylguanine, divalent 5,6-dihydrouracil, divalent 5-methylcytosine, or divalent 5-hydroxymethylcytosine. In embodiments, B is a divalent cytosine. In embodiments, B is a divalent guanine. In embodiments, B is a divalent adenine. In embodiments, B is a divalent thymine. In embodiments, B is a divalent uracil. In embodiments, B is a divalent hypoxanthine. In embodiments, B is a divalent xanthine. In embodiments, B is a deaza-adenine. In embodiments, B is a deaza-guanine. In embodiments, B is a deaza-hypoxanthine or a derivative thereof divalent 7-methylguanine. In embodiments, B is a divalent 5,6-dihydrouracil. In embodiments, B is a divalent 5-methylcytosine. In embodiments, B is a divalent 5-hydroxymethylcytosine.

In embodiments, B is a divalent cytosine or a derivative thereof. In embodiments, B is a divalent guanine or a derivative thereof. In embodiments, B is a divalent adenine or a derivative thereof. In embodiments, B is a divalent thymine or a derivative thereof. In embodiments, B is a divalent uracil or a derivative thereof. In embodiments, B is a divalent hypoxanthine or a derivative thereof. In embodiments, B is a divalent xanthine or a derivative thereof. In embodiments, B is a deaza-adenine or a derivative thereof. In embodiments, B is a deaza-guanine or a derivative thereof. In embodiments, B is a deaza-hypoxanthine or a derivative thereof divalent 7-methylguanine or a derivative thereof. In embodiments, B is a divalent 5,6-dihydrouracil or a derivative thereof. In embodiments, B is a divalent 5-methylcytosine or a derivative thereof. In embodiments, B is a divalent 5-hydroxymethylcytosine or a derivative thereof.

In embodiments, B is

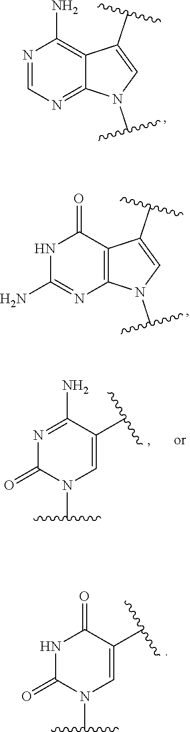 [SMILES: *c1cn(*)c2ncnc(N)c12 ; *c1cn(*)c2nc(N)[nH]c(=O)c12 ; *c1cn(*)c(=O)nc1N ; *c1cn(*)c(=O)[nH]c1=O]

In embodiments, B is

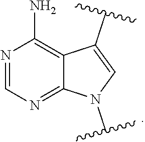 [SMILES: *c1cn(*)c2ncnc(N)c12]

In embodiments, B is

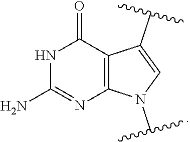 [SMILES: *c1cn(*)c2nc(N)[nH]c(=O)c12]

In embodiments, B is

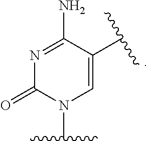 [SMILES: *c1cn(*)c(=O)nc1N]

In embodiments, B is v

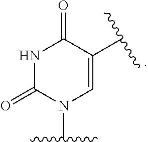 [SMILES: *c1cn(*)c(=O)[nH]c1=O]

In a further embodiment of any of the inventions described herein, the dNTP analogue or rNTP analogue has the structure:

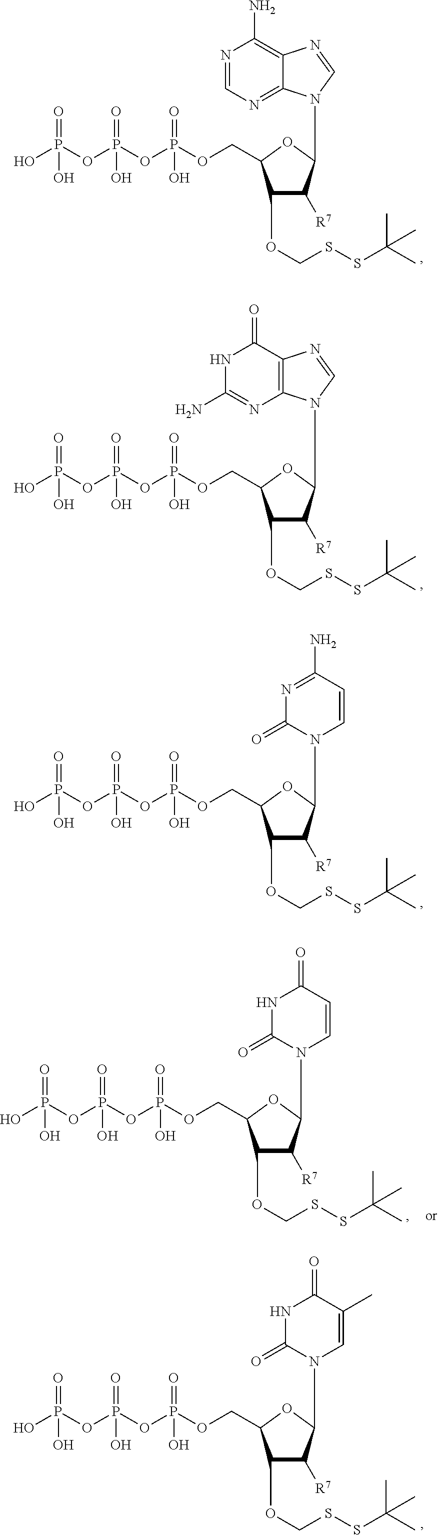

wherein R′ is H or OH.

In one embodiment of any of the inventions described herein, the DNA or RNA is in a solution in a reaction chamber disposed on a sensor which is (i) formed in a semiconductor substrate and (ii) comprises a field-effect transistor or chemical field-effect transistor configured to provide at least one output signal in response to an increase in hydrogen ion concentration of the solution resulting from the formation of a phosphodiester bond between a nucleotide triphosphate or nucleotide triphosphate analogue and a primer or a DNA or RNA extension product.

In another embodiment of any of the inventions described herein, the reaction chamber is one of a plurality of reaction chambers disposed on a sensor array formed in a semiconductor substrate and comprised of a plurality of sensors, each reaction chamber being disposed on at least one sensor and each sensor of the array comprising a field-effect transistor configured to provide at least one output signal in response to an increase in hydrogen ion concentration of the solution resulting from the formation of a phosphodiester bond between a nucleotide triphosphate or nucleotide triphosphate analogue and a primer or a DNA or RNA extension product.

In another embodiment of any of the inventions described herein, the reaction chamber is one of a plurality of reaction chambers disposed on a sensor array formed in a semiconductor substrate and comprised of a plurality of sensors, each reaction chamber being disposed on at least one sensor and each sensor of the array comprising a chemical field-effect transistor configured to provide at least one output electrical signal in response to an increase in hydrogen ion concentration of the solution resulting from the formation of a phosphodiester bond between a nucleotide triphosphate or nucleotide triphosphate analogue and a primer or a DNA or RNA extension product. In another embodiment, said sensors of said array each occupy an area of 100 μm or less and have a pitch of 10 μm or less and wherein each of said reaction chambers has a volume in the range of from 1 μm³ to 1500 μm³. In another embodiment, each of said reaction chambers contains at least 105 copies of the single-stranded DNA or RNA in the solution. In another embodiment, said plurality of said reaction chambers and said plurality of said sensors are each greater in number than 256,000.

In another embodiment of any of the inventions described herein, single-stranded DNA(s) or RNA(s) in the solution are attached to a solid substrate. In an embodiment, the single-stranded DNA or RNA or primer is attached to a solid substrate via a polyethylene glycol molecule. In a further embodiment, the solid substrate is azide-functionalized. In an embodiment, the DNA or RNA or primer is attached to a solid substrate via an azido linkage, an alkynyl linkage, or biotin-streptavidin interaction. In an embodiment, the DNA or RNA or primer is alkyne-labeled.

In another embodiment of any of the inventions described herein, the DNA or RNA or primer is attached to a solid substrate which is in the form of a chip, a bead, a well, a capillary tube, a slide, a wafer, a filter, a fiber, a porous media, a matrix, a porous nanotube, or a column. In another embodiment, the DNA or RNA or primer is attached to a solid substrate which is a metal, gold, silver, quartz, silica, a plastic, polypropylene, a glass, nylon, or diamond. In another embodiment, the DNA or RNA or primer is attached to a solid substrate which is a porous non-metal substance to which is attached or impregnated a metal or combination of metals. In another embodiment, the DNA or RNA or primer is attached to a solid substrate which is in turn attached to a second solid substrate. In a further embodiment, the second solid substrate is a chip.

In another embodiment of any of the inventions described herein, 1×10⁹ or fewer copies of the DNA or RNA or primer are attached to the solid substrate. In further embodiments, 1×10⁸ or fewer, 2×10⁷ or fewer, 1×10⁷ or fewer, 1×10⁶ or fewer, 1×10⁴ or fewer, or 1,000 or fewer copies of the DNA or RNA or primer are attached to the solid substrate.

In another embodiment of any of the inventions described herein, 10,000 or more copies of the DNA or RNA or primer are attached to the solid substrate. In further embodiments, 1×10⁷ or more, 1×10⁸ or more, or 1×10⁹ or more copies of the DNA or RNA or primer are attached to the solid substrate.

In another embodiment of any of the inventions described herein, the DNA or RNA or primer are separated in discrete compartments, wells, or depressions on a solid surface.

In one embodiment, the method is performed in parallel on a plurality of single-stranded DNAs or RNAs. In another embodiment, the single-stranded DNAs or RNAs are templates having the same sequence. In another embodiment, the method further comprises contacting the plurality of single-stranded DNAs or RNAs or templates after the residue of the nucleotide residue has been determined in step (b), or (c), as appropriate, with a dideoxynucleotide triphosphate which is complementary to the nucleotide residue which has been identified, so as to thereby permanently cap any unextended primers or unextended DNA or RNA extension products.

In an embodiment of any of the methods described herein, the single-stranded DNA or RNA is amplified from a sample of DNA or RNA prior to step (a). In a further embodiment the single-stranded DNA or RNA is amplified by reverse transcriptase polymerase chain reaction.

In an embodiment of any of the inventions described herein, UV light is used to treat the R′ group of a dNTP analogue incorporated into a primer or DNA or RNA extension product so as to photochemically cleave the moiety attached to the 3′-O so as to replace the 3‘-O-R’ with a 3′-OH. In a further embodiment, the moiety is a 2-nitrobenzyl moiety.

In an embodiment of any of the inventions described herein, tris-(2-carboxyethyl)phosphine (TCEP) or tris(hydroxypropyl)phosphine (THP) is used to treat the R′ group of a dNTP or rNTP analogue incorporated into a primer or DNA or RNA extension product, so as to cleave the moiety attached to the 3′-O so as to replace the 3‘-O-R’ with a 3′-OH. In a further embodiment, the moiety is a dithio moiety. In yet a further embodiment, the dithio moiety is an alkyldithiomethyl moiety. In yet a further embodiment, the alkyldithiomethyl moiety is independently selected from the group consisting of methyldithiomethyl, ethyldithiomethyl, propyldithiomethyl, isopropyldithiomethyl, butyldithiomethyl, t-butyldithiomethyl, and phenyldithiomethyl. In one embodiment of the invention, the alkyldithiomethyl moiety is a t-butyldithiomethyl moiety.

Examples of attaching nucleic acids to solid substrates, or immobilization of nucleic acids, are described in Immobilization of DNA on Chips II, edited by Christine Wittmann (2005), Springer Verlag, Berlin, which is hereby incorporated by reference. Ion sensitive field effect transistors (FET) and methods and apparatus for measuring H⁺ generated by sequencing by synthesis reactions using large scale FET arrays are known in the art and described in U.S. Patent Application Publication Nos. US 20100035252, US 20100137143, US 20100188073, US 20100197507, US 20090026082, US 20090127589, US 20100282617, US 20100159461, US20080265985, US 20100151479, US 20100255595, U.S. Pat. Nos. 7,686,929 and 7,649,358, and PCT International Publication Nos. WO/2009/158006 A3, WO/2008/076406 A2, WO/2010/008480 A2, WO/2010/008480 A3, WO/2010/016937 A2, WO/2010/047804 A1, and WO/2010/016937 A3, the contents of each of which are hereby incorporated by reference in their entirety.

As used herein, “hydrocarbon” refers to a compound containing hydrogen and carbon. A “hydrocarbyl” refers to a hydrocarbon which has had one hydrogen removed. Hydrocarbyls may be unsubstituted or substituted. For example, hydrocarbyls may include alkyls (such as methyl or ethyl), alkenyls (such as ethenyl and propenyl), alkynyls (such as ethynyl and propynyl), and phenyls (such as benzyl).

As used herein, “alkyl” includes both branched and straight-chain saturated aliphatic hydrocarbon groups having the specified number of carbon atoms and may be unsubstituted or substituted. Thus, C₁-Cn as in “C₁-Cn alkyl” is defined to include groups having 1, 2, . . . , n−1 or n carbons in a linear or branched arrangement. For example, a “C₁-C₅ alkyl” is defined to include groups having 1, 2, 3, 4, or 5 carbons in a linear or branched arrangement, and specifically includes methyl, ethyl, n-propyl, isopropyl, n-butyl, t-butyl, and pentyl. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

As used herein, “alkenyl” refers to a non-aromatic hydrocarbon radical, straight or branched, containing at least 1 carbon to carbon double bond, and up to the maximum possible number of non-aromatic carbon-carbon double bonds may be present, and may be unsubstituted or substituted. For example, “C₂-C₅ alkenyl” means an alkenyl radical having 2, 3, 4, or 5, carbon atoms, and up to 1, 2, 3, or 4, carbon-carbon double bonds respectively. Alkenyl groups include ethenyl, propenyl, and butenyl.

As used herein, “alkynyl” refers to a hydrocarbon radical straight or branched, containing at least 1 carbon to carbon triple bond, and up to the maximum possible number of non-aromatic carbon-carbon triple bonds may be present, and may be unsubstituted or substituted. Thus, “C₂-C₅ alkynyl” means an alkynyl radical having 2 or 3 carbon atoms and 1 carbon-carbon triple bond, or having 4 or 5 carbon atoms and up to 2 carbon-carbon triple bonds. Alkynyl groups include ethynyl, propynyl and butynyl.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N-OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl” by itself or in combination with another term, means, unless otherwise stated, a heteroalkyl including at least one triple bond. A heteroalkynyl may optionally include more than one triple bond and/or one or more double bonds in additional to the one or more triple bonds.

The symbol “ [symbol] ” denotes the point of attachment of a chemical moiety to the remainder of a molecule or chemical formula.

“Alkyldithiomethyl” refers to a compound, or portion thereof, comprising a dithio group, where one of the sulfurs is directly connected to a methyl group and the other sulfur is directly connected to an alkyl group. An example is the structure

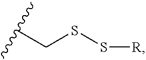

wherein R is an alkyl group and the wavy line represents a point of connection to another portion of the compound. In some cases, the alkyldithiomethyl is methyldithiomethyl, ethyldithiomethyl, propyldithiomethyl, isopropyldithiomethyl, butyldithiomethyl, t-butyldithiomethyl, and phenyldithiomethyl.

As used herein, “substituted” refers to a functional group as described above such as an alkyl, or a hydrocarbyl, in which at least one bond to a hydrogen atom contained therein is replaced by a bond to non-hydrogen or non-carbon atom, provided that normal valencies are maintained and that the substitution(s) result(s) in a stable compound. Substituted groups also include groups in which one or more bonds to a carbon(s) or hydrogen(s) atom are replaced by one or more bonds, including double or triple bonds, to a heteroatom. Non-limiting examples of substituents include the functional groups described above, —NO₂, and, for example, N, e.g. so as to form —CN.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl. A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkelyene (e.g., alkylene, alkenylene, or alkynylene) is a substituted or unsubstituted C₁-C₂₀ alkylene, each substituted or unsubstituted heteroalkelyene is a substituted or unsubstituted 2 to 20 membered heteroalkylene, each substituted or unsubstituted cycloalkelyene is a substituted or unsubstituted C₃-C₈ cycloalkylene, each substituted or unsubstituted heterocycloalkelyene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkelyene (e.g., alkylene, alkenylene, or alkynylene) is a substituted or unsubstituted C₁-C₈ alkylene, each substituted or unsubstituted heteroalkelyene is a substituted or unsubstituted 2 to 8 membered heteroalkylene, each substituted or unsubstituted cycloalkelyene is a substituted or unsubstituted C₃-C₇ cycloalkylene, each substituted or unsubstituted heterocycloalkelyene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

As disclosed herein, and unless stated otherwise, each of the following terms shall have the definition set forth below.

### A—Adenine;

### C—Cytosine;

DNA—Deoxyribonucleic acid;

### G—Guanine;

RNA—Ribonucleic acid;

### T—Thymine;

### U—Uracil; and

### NRT—Nucleotide Reversible Terminator.

“Nucleic acid” shall mean, unless otherwise specified, any nucleic acid molecule, including, without limitation, DNA, RNA and hybrids thereof. In an embodiment the nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA). In an embodiment the DNA or RNA is not modified. In an embodiment the DNA or RNA is modified only insofar as it is attached to a surface, such as a solid surface.

“Solid substrate” or “solid support” shall mean any suitable medium present in the solid phase to which a nucleic acid or an agent may be affixed. Non-limiting examples include chips, beads, nanopore structures and columns. In an embodiment the solid substrate or solid support can be present in a solution, including an aqueous solution, a gel, or a fluid.

“Hybridize” shall mean the annealing of one single-stranded nucleic acid to another nucleic acid based on the well-understood principle of sequence complementarity. In an embodiment the other nucleic acid is a single-stranded nucleic acid. The propensity for hybridization between nucleic acids depends on the temperature and ionic strength of their milieu, the length of the nucleic acids and the degree of complementarity. The effect of these parameters on hybridization is well known in the art (see Sambrook J, Fritsch E F, Maniatis T. 1989. Molecular cloning: a laboratory manual. Cold Spring Harbor Laboratory Press, New York.). As used herein, hybridization of a primer sequence, or of a DNA extension product, to another nucleic acid shall mean annealing sufficient such that the primer, or DNA extension product, respectively, is extendable by creation of a phosphodiester bond with an available nucleotide or nucleotide analogue capable of forming a phosphodiester bond.

As used herein, unless otherwise specified, a base of a nucleotide or nucleotide analogue which is a “different type of base from the type of base” (of a reference) means the base has a different chemical structure from the other/reference base or bases. For example, a base that is “different from” adenine would include a base that is guanine, a base that is uracil, a base that is cytosine, and a base that is thymine. For example, a base that is “different from” adenine, thymine, and cytosine would include a base that is guanine and a base that is uracil.

As used herein, “primer” (a primer sequence) is a short, often chemically synthesized, oligonucleotide of appropriate length, for example about 18-24 bases, sufficient to hybridize to a target nucleic acid (e.g. a single-stranded nucleic acid) and permit the addition of a nucleotide residue thereto, or oligonucleotide or polynucleotide synthesis therefrom, under suitable conditions well-known in the art. The target nucleic acid may be self-priming. In an embodiment the primer is a DNA primer, i.e. a primer consisting of, or largely consisting of deoxyribonucleotide residues. In another embodiment the primer is an RNA primer, i.e. a primer consisting of, or largely consisting of ribonucleotide residues. The primers are designed to have a sequence which is the reverse complement of a region of template/target DNA or RNA to which the primer hybridizes. The addition of a nucleotide residue to the 3′ end of a DNA primer by formation of a phosphodiester bond results in the primer becoming a “DNA extension product.” The addition of a nucleotide residue to the 3′ end of the DNA extension product by formation of a phosphodiester bond results in a further DNA extension product. The addition of a nucleotide residue to the 3′ end of an RNA primer by formation of a phosphodiester bond results in the primer becoming an “RNA extension product.” The addition of a nucleotide residue to the 3′ end of the RNA extension product by formation of a phosphodiester bond results in a further RNA extension product. A “probe” is a primer with a detectable label or attachment.

As used herein a nucleic acid, such as a single-stranded DNA or RNA, “in a solution” means the nucleic acid is submerged in an appropriate solution. The nucleic acid in the solution may be attached to a surface, including a solid surface. Thus, as used herein, “in a solution”, unless context indicates otherwise, encompasses, for example, both a DNA free in a solution and a DNA in a solution wherein the DNA is tethered to a solid surface.

A “nucleotide residue” is a single nucleotide in the state it exists after being incorporated into, and thereby becoming a monomer of, a polynucleotide. Thus, a nucleotide residue is a nucleotide monomer of a polynucleotide, e.g. DNA, which is bound to an adjacent nucleotide monomer of the polynucleotide through a phosphodiester bond at the 3′ position of its sugar and is bound to a second adjacent nucleotide monomer through its phosphate group, with the exceptions that (i) a 3′ terminal nucleotide residue is only bound to one adjacent nucleotide monomer of the polynucleotide by a phosphodiester bond from its phosphate group, and (ii) a 5′ terminal nucleotide residue is only bound to one adjacent nucleotide monomer of the polynucleotide by a phosphodiester bond from the 3′ position of its sugar.

Because of well-understood base-pairing rules, determination of which dNTP or rNTP analogue is incorporated into a primer or DNA or RNA extension product thereby reveals the identity of the complementary nucleotide residue in the single-stranded polynucleotide that the primer or DNA or RNA extension product is hybridized to. Thus, if the dNTP analogue that was incorporated comprises an adenine, a thymine, a cytosine, or a guanine, then the complementary nucleotide residue in the single-stranded DNA is identified as a thymine, an adenine, a guanine or a cytosine, respectively. The purine adenine (A) pairs with the pyrimidine thymine (T). The pyrimidine cytosine (C) pairs with the purine guanine (G). Similarly, with regard to RNA, where the RNA is hybridized to an RNA primer, if the rNTP analogue that was incorporated comprises an adenine, a uracil, a cytosine, or a guanine, then the complementary nucleotide residue in the single-stranded RNA is identified as a uracil, an adenine, a guanine or a cytosine, respectively. Where the RNA is hybridized to a DNA primer, if the dNTP analogue that was incorporated comprises an adenine, a thymine, a cytosine, or a guanine, then the complementary nucleotide residue in the single-stranded RNA is identified as a uracil, an adenine, a guanine or a cytosine, respectively.

Incorporation into an oligonucleotide or polynucleotide (such as a primer or DNA or RNA extension strand) of a dNTP or rNTP analogue means the formation of a phosphodiester bond between the 3′ carbon atom of the 3′ terminal nucleotide residue of the polynucleotide and the 5′ carbon atom of the dNTP or rNTP analogue resulting in the loss of pyrophosphate from the dNTP or rNTP analogue.

As used herein, a deoxyribonucleotide triphosphate (dNTP) analogue, unless otherwise indicated, is a dNTP having substituted in the 3′—OH group of the sugar thereof, in place of the H atom of the 3′—OH group, or connected via a linker to the base thereof, a chemical group which is —CH₂N₃, or is a hydrocarbyl, or a substituted hydrocarbyl, having a mass of less than 300 daltons, or a dithio moiety, and which does not prevent the dNTP analogue from being incorporated into a polynucleotide, such as DNA, by formation of a phosphodiester bond. Similarly, a deoxyribonucleotide analogue residue is a deoxyribonucleotide analogue which has been incorporated into a polynucleotide and which still comprises its chemical group which is —CH₂N₃, or is a hydrocarbyl, or a substituted hydrocarbyl, having a mass of less than 300 daltons, or is a dithio moiety. In a preferred embodiment of the deoxyribonucleotide triphosphate analogue, the chemical group is substituted in the 3′—OH group of the sugar thereof, in place of the H atom of the 3′—OH group. In a preferred embodiment of the deoxyribonucleotide analogue residue, the chemical group is substituted in the 3′—OH group of the sugar thereof, in place of the H atom of the 3′—OH group.

As used herein, a ribonucleotide triphosphate (rNTP) analogue, unless otherwise indicated, is a rNTP having substituted in the 3′—OH group of the sugar thereof, in place of the H atom of the 3′—OH group, or connected via a linker to the base thereof, a chemical group which is —CH₂N₃, or is a hydrocarbyl, or a substituted hydrocarbyl, having a mass of less than 300 daltons, or is a dithio moiety, and which does not prevent the rNTP analogue from being incorporated into a polynucleotide, such as RNA, by formation of a phosphodiester bond. Similarly, a ribonucleotide analogue residue is a ribonucleotide analogue which has been incorporated into a polynucleotide and which still comprises its chemical group that is —CH₂N₃, or is a hydrocarbyl, or a substituted hydrocarbyl, having a mass of less than 300 daltons, or is a dithio moiety. In a preferred embodiment of the ribonucleotide triphosphate analogue, the chemical group is substituted in the 3′—OH group of the sugar thereof, in place of the H atom of the 3′—OH group. In a preferred embodiment of the ribonucleotide analogue residue, the chemical group is substituted in the 3′—OH group of the sugar thereof, in place of the H atom of the 3′—OH group.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing the compounds of the present invention, one of ordinary skill in the art will recognize that the various substituents, i.e. R₁, Rₓ, etc. are to be chosen in conformity with well-known principles of chemical structure connectivity.

It is understood that where radicals are represented herein by structure, the point of attachment to the main structure is represented by a wavy line.

In the compound structures depicted herein, hydrogen atoms, except on ribose and deoxyribose sugars, are generally not shown. However, it is understood that sufficient hydrogen atoms exist on the represented carbon atoms to satisfy the octet rule.

Where a range of values is provided, unless the context clearly dictates otherwise, it is understood that each intervening integer of the value, and each tenth of each intervening integer of the value, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range.

Where the stated range includes one or both of the limits, ranges excluding (i) either or (ii) both of those included limits are also included in the invention.

All combinations of the various elements described herein are within the scope of the invention. All sub-combinations of the various elements described herein are also within the scope of the invention.

This invention will be better understood by reference to the Experimental Details which follow, but those skilled in the art will readily appreciate that the specific experiments detailed are only illustrative of the invention as described more fully in the claims which follow thereafter.

### Experimental Details

There are a number of innovative aspects to the present invention. For example, the combination of the ion sensing strategy and the sequencing-by-synthesis approach using NRTs (Ju et al. 2003; Li et al. 2003; Ruparel et al. 2005; Seo et al. 2005; Ju et al. 2006) is a novel use of disparate sequencing paradigms to produce a hybrid approach that is very low cost, has good sensitivity, avoids false positive signals caused by spontaneous NTP depyrophosphorylation, and at the same time is as accurate as any of the available sequencing strategies.

Here it is disclosed that NRTs can be exploited for ion sensing SBS because: (1) NRTs display specificity and good processivity in polymerase extension; (2) NRTs permit the ion-sensing step to address single base incorporation, overcoming the complications of multiple base incorporation in homopolymer runs of different lengths; (3) synthesis of several alternative sets of NRTs with assorted blocking groups on the 3′-OH and elsewhere in the deoxyribose allows selection of the best NRTs with regard to speed and specificity of incorporation and ease of removal of the blocking group, while maintaining compatibility with DNA stability and ion sensing requirements (Li et al. 2003; Ruparel et al. 2005; Seo et al. 2005; Ju et al. 2006); (4) NRTs provide modified nucleotides that are identical to normal nucleotides after blocking group cleavage, thus allowing longer reads to be achieved; and (5) absence of fluorescent tags on the modified nucleotides increases polymerase incorporation efficiency, greatly lowering the cost of their synthesis, and removing the need to account for background fluorescence.

In the past, high-throughput DNA sequencing was accomplished by taking advantage of the automation possibilities afforded by the Sanger sequencing approach, relative to the competing chemical sequencing strategy (Sanger et al. 1977). Although use of 4-color fluorescent tags and capillary instruments enabled quite high throughput (Ju et al. 1995; Smith et al. 1986), up to >600-base reads every couple of hours per instrument, the DNA preparation procedures needed for whole genome sequencing were economically prohibitive, often necessitating DNA cloning and clone storage. Recent strategies utilizing either sequencing by synthesis (Roche pyrosequencing and Illumina instruments) or sequencing by hybridization and ligation (ABI's SOLID™ platform) have overcome this obstacle by taking advantage of variations on polony PCR (on beads or directly on sequencing chips) (Wheeler et al. 2008; Bentley et al. 2008; McKernan et al. 2009), and at the same time taken advantage of miniaturization strategies to allow millions of reads at the same time, dwarfing essentially all the advantages of the Sanger approach except its ability to generate fairly long reads. Still newer strategies endorsed by Helicos and Pacific Sciences have approached single-molecule sequencing, though at some cost to accuracy (Harris et al. 2008; Eid et al. 2008). Other options such as the use of nanopores to discriminate released nucleotides or the sequence of intact DNA chains are still being assessed (Branton et al. 2008).

For the sequencing by synthesis strategies, there are two general schemes that depend on the nature of the detection strategy. With detection of a single signal (light, a fluorescent dye, or a pH change in the case of Roche 454, Helicos, and Ion Torrent, respectively) upon the incorporation of each nucleotide, it is necessary to add each base one by one, and score the incorporation based on whether an output signal was generated. Such methods can reduce reagent cost and simplify the instrument design, but have lower overall accuracy. In contrast, methods that utilize multiple output signals (e.g. 4 fluorescent dyes, one for each of the bases of DNA), while involving more expensive reagents, can increase accuracy, particularly if background signals are reduced or computationally subtracted. Several of these methods, especially those of the first design, utilize standard dNTPs for incorporation and measure byproducts of the formation of the phosphodiester bond. A downside of this approach is difficulty in interpreting signals in homopolymer stretches. Even if only one of the dNTPs is added at a time, one must take into account the fact that if its complementary base is present at the next several positions, it would be important but difficult to determine exactly how many of the nucleotides were added in a row. The current protocols usually take additive measures of the signal, but beyond about 3 or 4 bases, it becomes difficult to distinguish base counts.

Here, it is disclosed that the use of 3′-O-modified nucleotide reversible terminators (NRTs) overcomes these problems.

Ion Sensing During Sequencing by Synthesis:

Recently, Ion Torrent, Inc. has introduced a sequencing method that leverages the enormous progress in the semiconductor field over the past decades. The method is based on the release of a H⁺ ion upon creation of the phosphodiester bond in the polymerase reaction. Reactions take place in a series of wells built into a chip, and a detection layer is attached to a semiconductor chip to directly convert the resulting pH change, a chemical signal, into digital data. This technology is rapid, inexpensive, highly scalable, and uses natural nucleotides. Because there is a single signal regardless of the nucleotide that gets incorporated, it is necessary to add the four nucleotides one at a time. This can lead to difficulty in interpreting signals in homopolymer stretches, places where a nucleotide will be incorporated multiple times in the same round of the reaction. This problem is solved herein by using specific NRTs, which have been successfully used as outlined hereinbelow.

Sequencing by Synthesis with Reversible Terminators:

A series of nucleotide reversible terminators (NRTs) to accomplish sequencing by synthesis has been described in numerous publications (Ju et al. 2006; Wu et al. 2007; Guo et al. 2008). In essence, this process involves the use of nucleotide analogues that have blocking groups at the 3′-OH position, which, once incorporated into DNA, prevent addition of the subsequent nucleotide. DNA templates are bound to a surface and primers are hybridized to these templates. One can then measure the incorporation of a particular NRT onto the priming strand, due to its complementarity to a nucleotide on the template strand, by virtue of specific fluorophores attached to each base. These blocking groups and fluorophores can be easily removed using chemical or photo-cleavage reactions that do not damage the DNA template or primer. In this way, additional rounds of incorporation, detection and cleavage can take place. These SBS reactions are accurate, show no dephasing (reading ahead or lagging), and have relatively low background due to misincorporated nucleotides or incomplete removal of dyes.

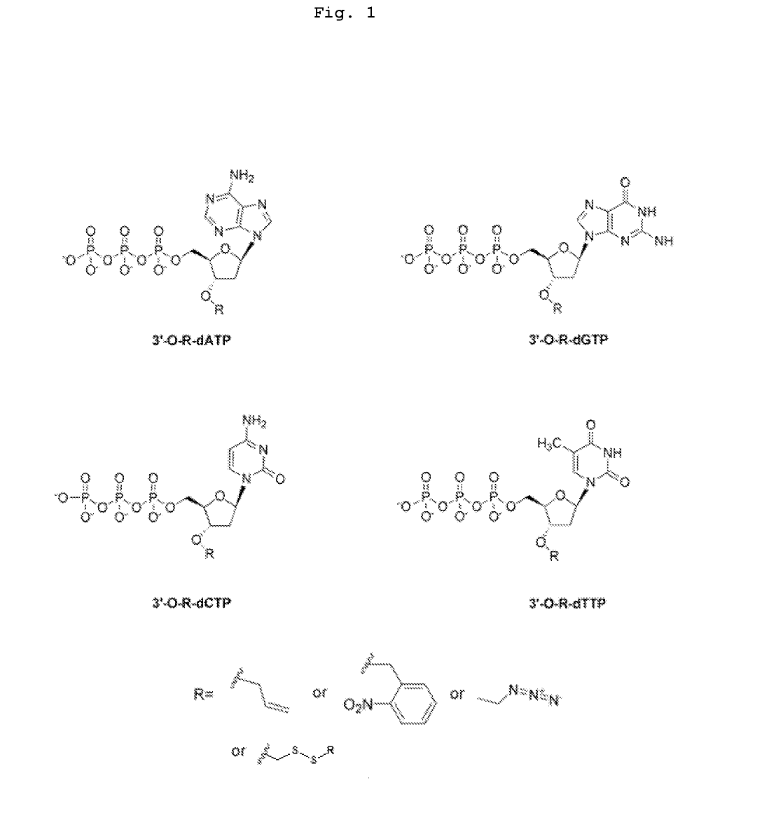

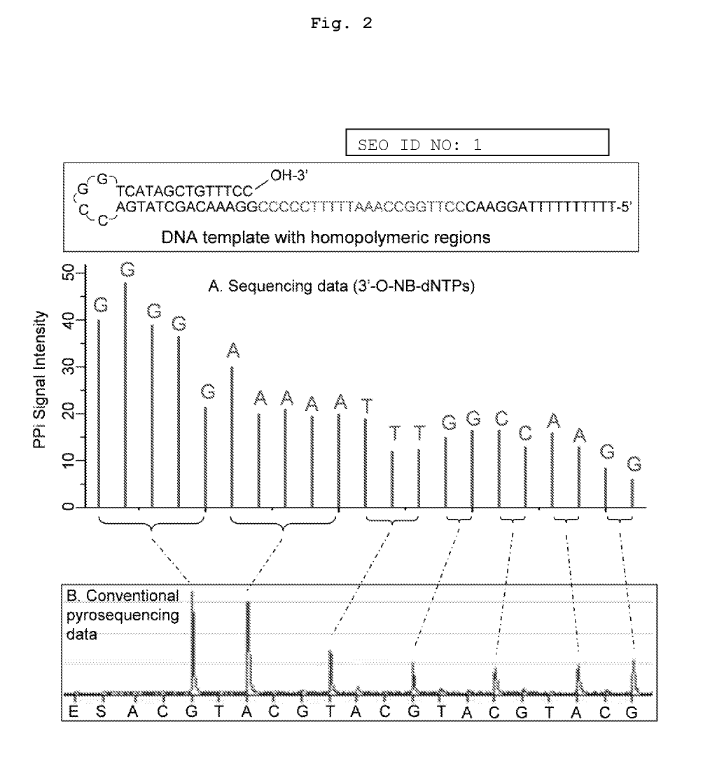

Four different sets of 4 NRTs (FIG. 1), bearing either an allyl, azidomethyl, dithiomethyl, or 2-nitrobenzyl group at the 3′-OH position, were synthesized and used to conduct pyrosequencing. While the 2-nitrobenzyl group could be cleaved by light (355 nm irradiation), simple chemicals were required to remove the allyl group (Na₂PdCl₄ plus trisodium triphenylphosphinetrisulfonate) or the azidomethyl group (Tris(2-carboxyethyl) phosphine) (Ju et al. 2006; Wu et al. 2007; Guo et al. 2008). Pyrosequencing was accomplished using each of these NRTs. Templates containing homopolymeric regions were immobilized on Sepharose beads, and extension-signal detection-deprotection cycles were conducted using the NRTs. As an example, pyrosequencing data using the NRTs modified by the photocleavable 2-nitrobenzyl group are shown in FIG. 2, and compared with conventional pyrosequencing using natural nucleotides. As can be seen, multiple-base signals that could not be easily discriminated by conventional pyrosequencing were easily resolved using the NRTs.

It is disclosed here that 3′-O-(2-nitrobenzyl) nucleotides are particularly useful for ion sensor measurement. They are quickly and efficiently incorporated, and photo-cleaved under conditions that do not require the presence of salts which could interfere with subsequent rounds of ion sensing. However, other modified bases are also useful. The 3′-O-azidomethyl group is particularly attractive. Not only is it efficiently incorporated, but it regenerates the natural base upon cleavage, thus does not impede subsequent nucleotide incorporation, resulting in long sequence reads (Guo et al. 2008).

Similarly, the 3′-O-dithiomethyl (3′-O-DTM) group is particularly attractive. It is disclosed herein that these nucleotide analogues are good terminators and substrates for DNA polymerase in a solution-phase DNA extension reaction and that the 3′-O-DTM group can be removed with high efficiency in a single step in aqueous solution. Moreover, the relatively small size of the 3′-O-DTM groups disclosed herein means that nucleotide analogues having these group are better polymerase substrates than other nucleotide analogues having bulky 3′-O-capping groups. The new DTM based linker after cleavage with THP or TCEP (tris(2-carboxyethyl)phosphine) does not require capping of the resulting free SH group as the cleaved product instantaneously collapses to the stable OH group. This is advantageous as cleavage of the disclosed 3′-O-DTM nucleotide analogues can occur efficiently under conditions compatible for polymerase reactions compatable for sequencing by synthesis.

Among the 3′-O-DTM nucleotide analogues disclosed herein are various nucleotide analogues having 3′-O-alkyldithiomethyl or 3′-O-t-butyldithiomethyl modifications. The utility of these types of molecules with a 3′-O-alkyldithiomethyl or 3′-O-t-butyldithiomethyl modification in Ion Sensor Sequencing by Synthesis has not been reported, but is herein disclosed. It is also disclosed herein that nucleotide polymerases will readily incorporate nucleotide analogues having 3′-O-alkyldithiomethyl or 3′-O-t-butyldithiomethyl modifications into a growing oligonucleotide during sequencing by synthesis, and reversibly terminate synthesis.

Preparation of a Library of NRTs and their Evaluation in SBS Polymerase and NRT Conditions Compatible with Ion Sensing.

Preparation of Full Sets of NRTs Sufficient for all Studies in this Application:

Established methods are used to synthesize the NRTs for ion-sensing SBS evaluation (Ju et al. 2003; Ju et al. 2006; Wu et al. 2007; Guo et al. 2008).

Characterization of Utility of NRTs for Ion Sensing:

The ion dependence for 9° N, Therminator II and Therminator III polymerases (all available from New England Biolabs, Ipswich, Mass.) that support incorporation of the NRTs are determined, initially using dideoxynucleotide triphosphates (ddNTPs) for single base extension reactions. Tests are performed in solution using synthetic template/primer systems, and cleaned-up extension products subjected to MALDI-TOF mass spectroscopy (MS) to quantify product yield. A series of monovalent and divalent cation, and monovalent anion concentrations, are tested. Once the basic parameters are established with dNTPs and ddNTPs, similar assays are performed using 3′-O-(2-nitrobenzyl), 3′-O-azidomethyl, 3′-O-DTM, and 3′-O-allyl nucleotides, utilizing enzymes that are best able to incorporate each of these modified nucleotides. Relevant time points are used to assess the salt dependence. While the salt-independent photo-cleavage of the 2-nitrobenzyl group may have advantages for the Ion Torrent-type system, automating chemical cleavage with azidomethyl, dithiomethyl, or allyl derivatives is also possible. Tris-(2-carboxyethyl)phosphine or tris(hydroxypropyl)phosphine may chemically cleave the dithio bond in dithiomethyl derivatives.

To test polymerase specificity in the low salt buffer systems, all four ddNTPs or ddNTP analogues are combined in the reactions. In a synthetic template-primer system it is already known which of the 4 bases should be added next, and these can each be distinguished as well-separated peaks in the mass spectra. By including two or more of the same base in a row, these spectra are examined to confirm that reactions are terminated completely after the first base. Next, the buffer system used is tested with each of the preferred polymerase/nucleotide reversible terminator combinations. Reduction of the salt concentration to low enough amounts to permit subsequent ion sensing is also tested.

NRTs Tested in Ion Sensing Platform.

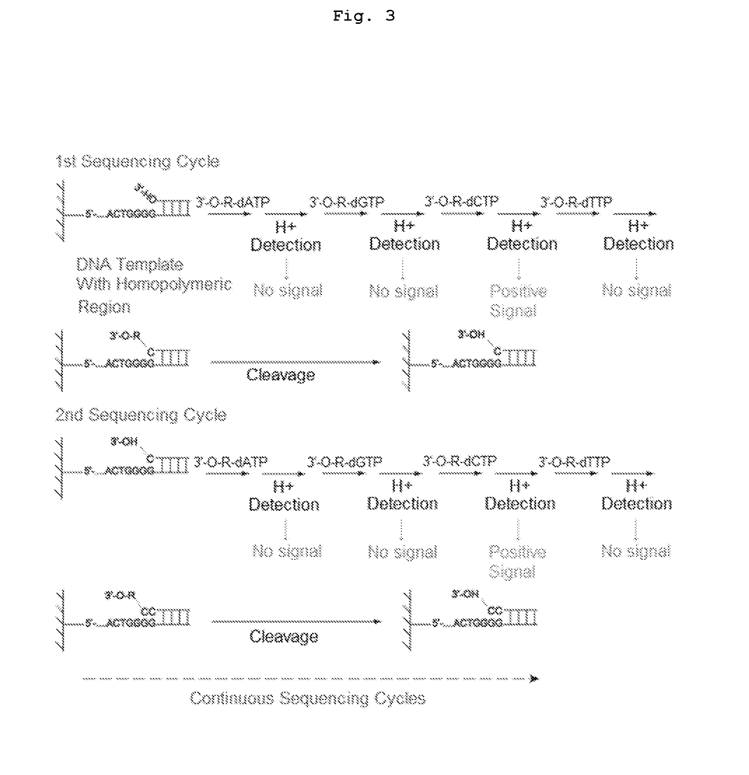

When enzyme/NRT/low ion buffer systems are established, short runs of 2 or 3 base extensions are conducted on an H⁺ sensitive ion sensing system, such as the Ion Torrent, Inc. platform, as outlined in FIG. 3. There is great flexibility in the number of samples that can be processed. Initially just a few different synthetic templates are employed. A range of the best buffer/salt conditions are used to maximize yields for ample detection by the ion sensor. Longer runs requiring larger amounts of NRTs are carried out under conditions giving the best results for the short runs. Templates can be attached to beads or directly to wells, and appropriate adapters are ligated if necessary to permit this. Artificial templates can be designed to test for specificity, dephasing (incomplete reactions or read-ahead), and ability to deal with long homopolymer sequences.

Ion Sensor SBS with NRTs.

After confirmation that the ion sensing system handles a set of NRTs with good efficiency, a biological sample (a known viral or a bacterial genome) is sequenced using the combined SBS-ion sensing approach. Sequences are assembled and searched for the presence of polymorphisms or sequence errors. For example, pathogenic and non-pathogenic Legionella species can be used and a comparative analysis performed, with gene annotation as necessary.

The accuracy for homopolymer runs of more than a few bases is near perfect with the NRTs, but much lower with standard nucleotides. The need for cycles of incorporation, detection and cleavage adds additional time, but with automation and maximized efficiencies of both incorporation and deprotection, this does not outweigh the gain in accuracy. A ddNTP synchronization step can be included optionally in each or every other cycle. A sequence is assembled de novo for a low-repeat bacterial sequence. With appropriate long-range mate-pair library preparation methods, de novo and re-sequencing of eukaryotic genomes is also possible. Both long and short sequence reads are usable and the method can be employed for conducting comparative sequence analysis, genome assembly, annotation, and pathway analysis for prokaryotic and eukaryotic species.

Rationale, Survey, Synthesis, and Use of 3′-O-Alkyldithiomethyl Analogues.

Various 3′-O-alkyldithiomethyl based modifications on nucleosides have been reported (Kwiatkowski 2007; Muller et al. 2011; Semenyuk et al. 2006) for the synthesis of oligonucleotides but their utility in DNA sequencing applications have not been reported.

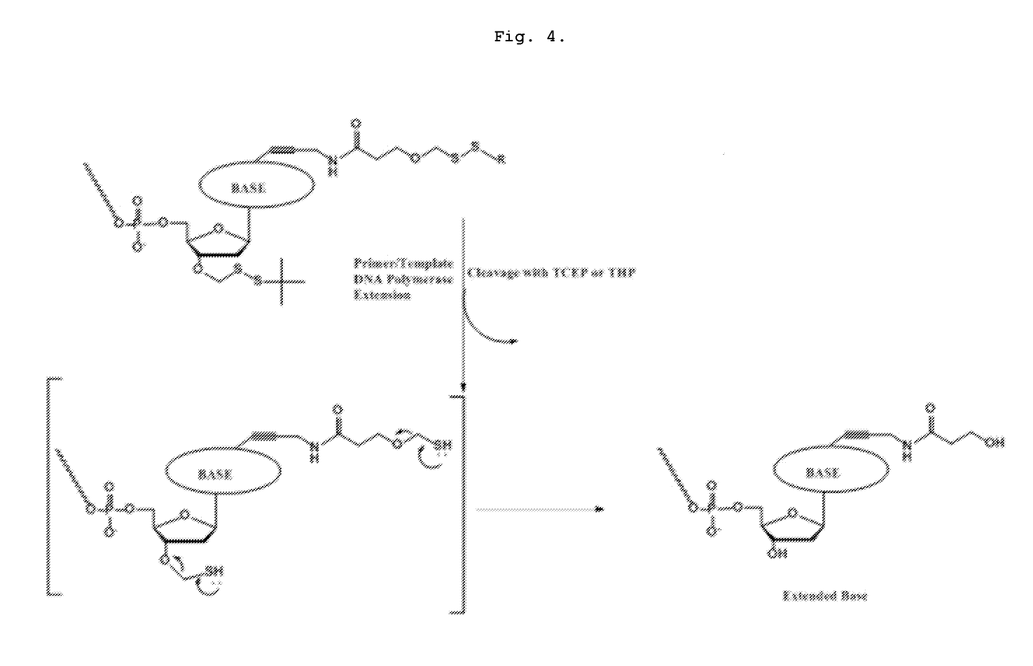

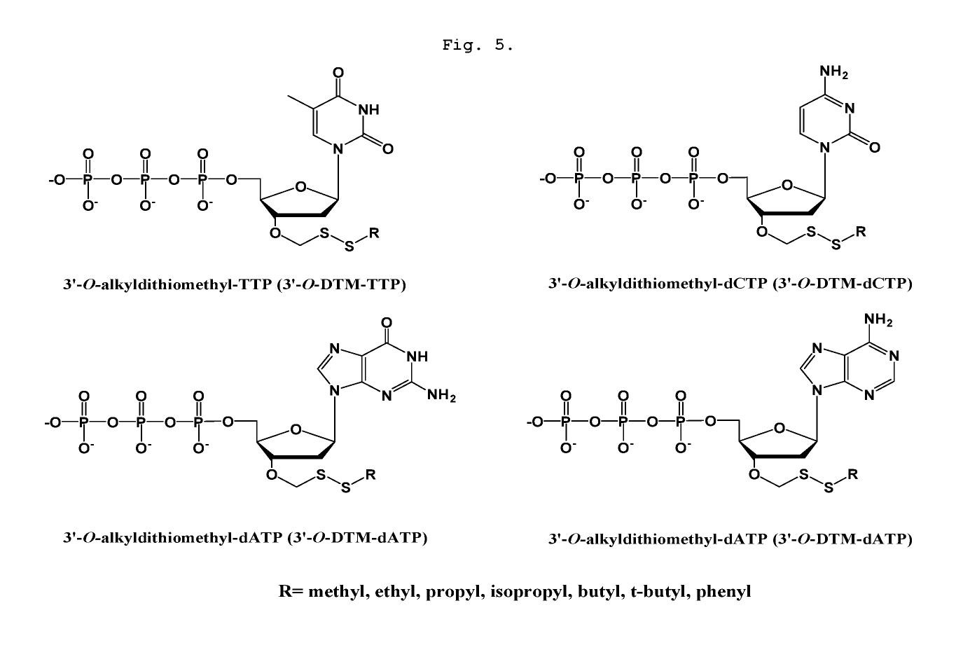

The design and synthesis of four chemically cleavable nucleotide analogues as reversible terminators for SBS is reported. Each of the nucleotide analogues contains a 3′-O-DTM group. It is disclosed herein that these nucleotide analogues are good terminators and substrates for DNA polymerase in a solution-phase DNA extension reaction and that the 3′-O-DTM group can be removed with high efficiency in a single step in aqueous solution. The new DTM based linker after cleavage with THP does not require capping of the resulting free SH group as the cleaved product instantaneously collapses to the stable OH group. This mechanism is shown in FIG. 4. Four 3′-O-alkyldithiomethyl-dNTPs are shown in FIG. 5.

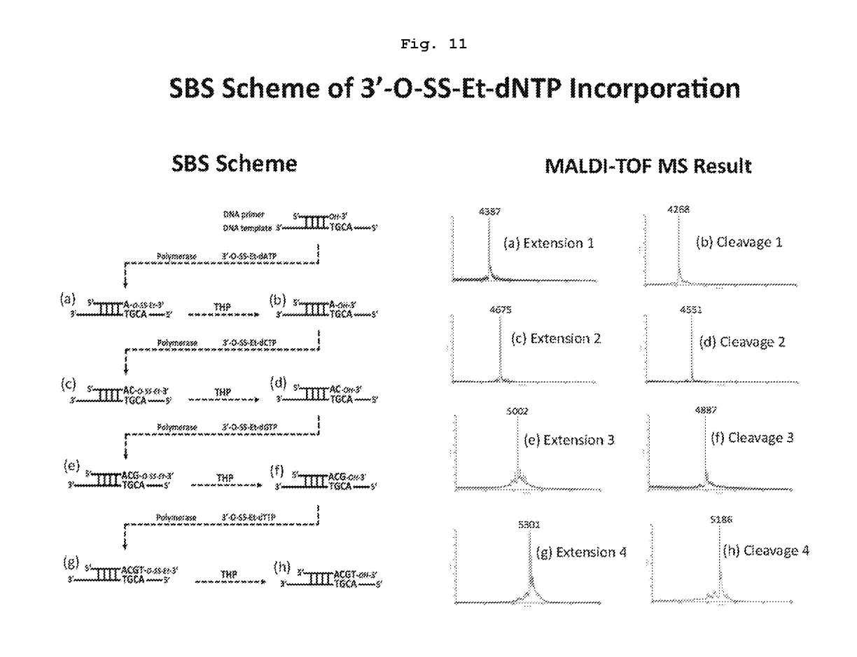

### Continuous Polymerase Extension Using 3′-O-Et-Dithiomethyl-dNTPs and Characterization by MALDI-TOF Mass Spectrometry (FIG. 11)

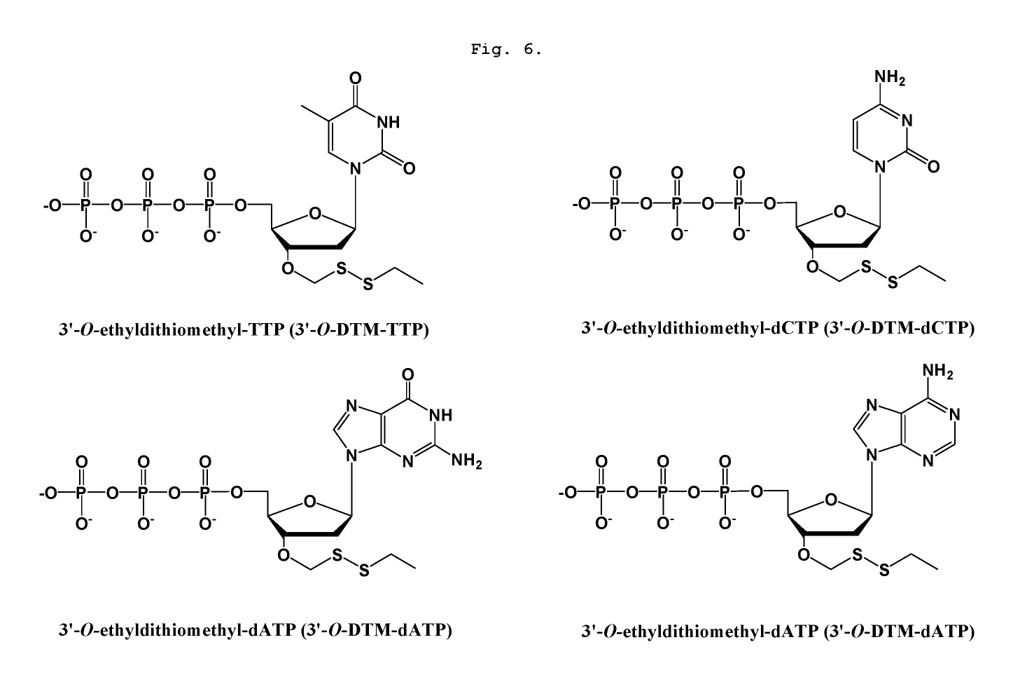

Continuous DNA sequencing by synthesis (FIG. 11, left) using four 3′-O-Et-dithiomethyl-dNTPs reversible terminators (3′-O-SS-Et-dNTPs or 3′-O-DTM-dNTPs)(Structures in FIG. 6) and MALDI-TOF MS spectra (right) obtained from each step of extension and cleavage. THP=(tris(hydroxypropyl)phosphine). The masses of the expected extension products are 4381, 4670, 4995, and 5295 Da respectively. The masses of the expected cleavage products are 4272, 4561, 4888, and 5186 Da. The measured masses shown (FIG. 11, right) are within the resolution of MALDI-TOF MS.

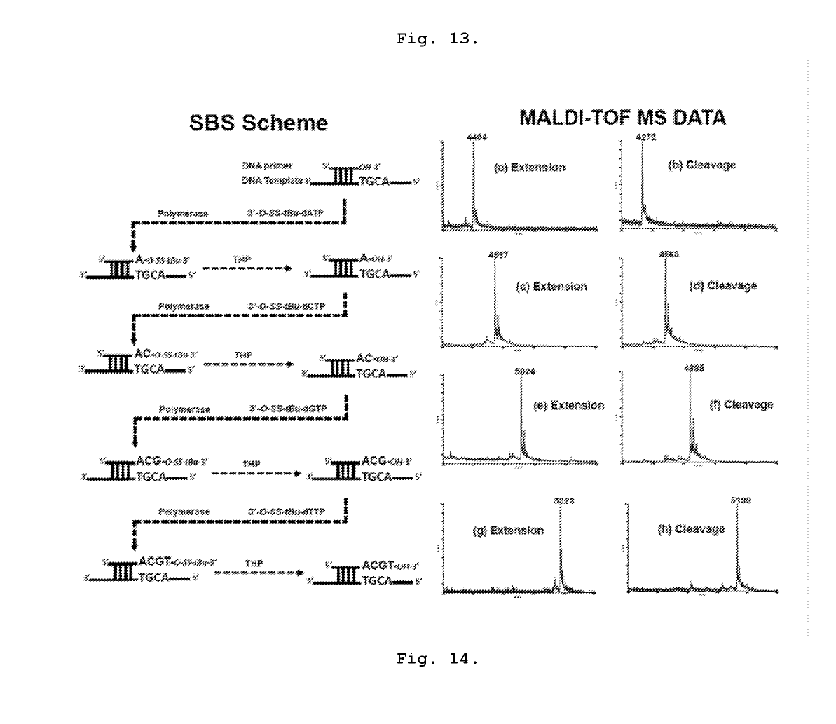

### Continuous Polymerase Extension Using 3′-O-t-Butyl-SS-dNTPs and Characterization by MALDI-TOF Mass Spectrometry (FIG. 13)

To verify that nucleotide analogues having 3′-O-DTM-dNTPs are incorporated accurately in a base-specific manner in the polymerase reaction, four consecutive DNA extension and cleavage reactions were carried out in solution with 3′-O-DTM-dNTPs as substrates. This allowed the isolation of the DNA product at each step for detailed molecular structure characterization.

A complete consecutive 4-step SBS reaction was performed, which involved incorporation of each complementary 3′-O-DTM-dNTP, followed by MALDI-TOF MS analysis for sequence determination, and cleavage of the 3′-O-DTM blocking group from the DNA extension product to yield a free 3′—OH group for incorporating the next nucleotide analogue. A template-primer combination was designed in which the next four nucleotides to be added were A, C, G and T. As shown in FIG. 13, the SBS reaction was initiated with the 13-mer primer annealed to a DNA template. When the first complementary nucleotide, 3′-O-t-Butyl-SS-dATP (3′-O-DTM-dATP), was used in the polymerase reaction, it was incorporated into the primer to form a DNA extension product with a molecular weight of 4404 Daltons (Da) as confirmed by MALDI-TOF MS with the appearance of a single peak (FIG. 13, Top left). These results indicated that the 3′-O-DTM-dATP was quantitatively incorporated into the 13-mer DNA primer. After THP treatment to remove the DTM group from the DNA product and HPLC purification, the cleavage was confirmed by the presence of a single MS peak at 4272 Da, corresponding to the DNA product with the 3′-O-DTM group removed (FIG. 13, Top right). The newly formed DNA extension product with a free 3′—OH group was then used in a second polymerase reaction to incorporate a 3′-O-tButyl-SS-dCTP (3′-O-DTM-dCTP) which gave a single MS peak at 4697 Da (FIG. 13), indicating incorporation of a 3′-O-DTM-dCTP into the growing DNA strand in this cycle. After THP treatment, a single MS peak of the cleaved DNA product appeared at 4563 Da (FIG. 13), which demonstrated the complete removal of the DTM group from the DNA extension product.

The third incorporation was with 3′-O-t-Butyl-SS-dGTP (3′-O-DTM-dGTP); accurate masses of the corresponding DNA products were obtained by MALDI-TOF MS for the third nucleotide incorporation (5024 Da, FIG. 13, and cleavage reaction (4888 Da, FIG. 13). Finally, 3′-O-t-Butyl-SS-dTTP (3′-O-DTM-dTTP) incorporation in the fourth cycle and a final removal of the DTM group by THP was verified, as appropriate masses for the corresponding DNA products were obtained by MALDI-TOF MS for the fourth nucleotide incorporation (5328 Da, FIG. 13) and cleavage reaction (5199 Da, FIG. 13). These results demonstrate that all four 3′-O-DTM-dNTPs are efficiently incorporated base-specifically as reversible terminators into the growing DNA strand in a continuous polymerase reaction, and that the 3′-OH capping group on the DNA extension products is quantitatively cleaved by THP.

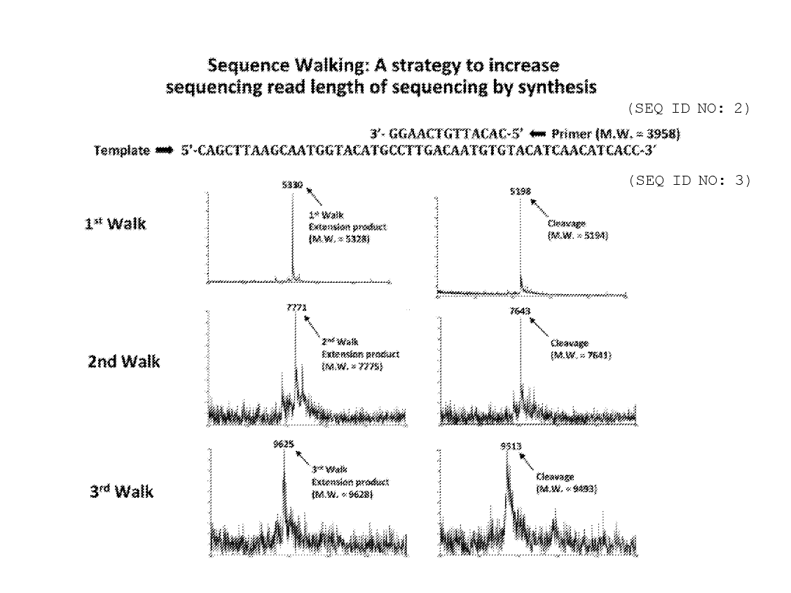

### Experiment Demonstrating Walking in Solution Using Three Natural dNTPs (dATP, dCTP and dTTP) and One 3′-O-t-Butyl-SS-dNTP (3′-O-DTM-dGTP) (FIG. 14)

We carried out a series of 3 walking steps using dATP, dCTP, dTTP and 3′-O-t-butyl-SS-dGTP. The results are presented in FIG. 14. WT49G (SEQ ID NO: 3) (5′-CAGCTTAAGCAATGGTACA TGCCTTGACAATGTGTACATCAACATCACC-3′) was designed as template for a 1st walk extension of 4 bases on the primer (SEQ ID NO: 2)(13mer, 5′-CACATTGTCAAGG-3′), 8 base extension in the 2^{nd} walk and 6 base extension in the 3^{rd} walk; in each case, the reaction will stop at the first corresponding C on the template (shown in red from right to left in the template). The WT49G template and 13mer primer were designed for efficient characterization of walking by MALDI-TOF mass spectrometry.

The reaction (50 μl) was carried out using 1 μmol of reversible terminator, 1 μmol of dATP, dCTP and dTTP, 500 μmol of primer (M.W. 3939), 5 units of Therminator IX DNA Polymerase (NEB), 300 μmol of WT49G in a 5 μl buffer containing 20 mM Tris-HCl, 10 mM (NH₄)₂SO₄, 10 mM KCl, 2 mM MgSO₄, 0.1% Triton X-100, pH 8.8 @ 25° C., and 100 μmol MnCl₂. The reactions were conducted in an ABI GeneAmp PCR System 9700 with initial incubation at 65° C. for 30 seconds, followed by 38 cycles of 65° C./30 sec, 45° C./30 sec, 65° C./30 sec. the reaction mixtures were desalted using Oligo Clean & Concentrator™ (ZYMO Research) and analyzed by MALDI-TOF MS (ABI Voyager DE). The cleavage reaction was carried out using THP at a final concentration of 5 mM incubated at 65° C. for 5 minutes, then the reaction mixtures were desalted using oligo Clean & Concentrator™ (ZYMO Research) and analyzed by MALDI-TOF MS. The results of each individual extension and cleavage are shown in FIG. 14.

After the first walk, the primer was extended to the point of the next C in the template (rightmost C highlighted in red in the template strand). The size of the extension product was 5330 Daltons (5328 Da expected) as shown in the top left MALDI-TOF MS trace. After cleavage with THP, the 5198 Da product shown at the top right was observed (5194 Da expected). A second walk was performed using this extended and cleaved primer, again using Therminator IX DNA polymerase, dATP, dCTP, dTTP and 3′-O-t-butyl-dGTP, to obtain the product shown in the middle left trace (7771 Da observed, 7775 Da expected to reach the middle C highlighted in red). After cleavage, a product of 7643 Da was obtained (expected 7641 Da). Finally a third walk and cleavage using the previously extended and cleaved primer were performed, giving products of 9625 Da (9628 Da expected to extend to the leftmost red highlighted C) and 9513 Da (9493 Da expected), respectively. The amount of nucleotides was adjusted in each walk according to extension length (2 μmol in 2^{nd} walk, 1.5 μmol in 3^{rd} walk) This demonstrates the ability to use a 3′-O-t-butyl nucleotide as a terminator for walking reactions.

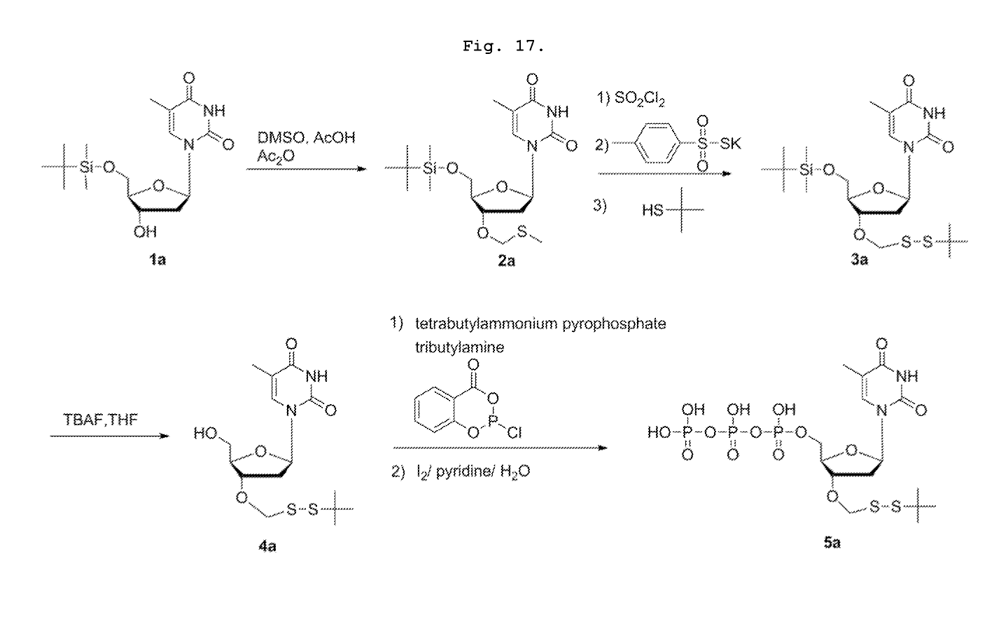

### Synthesis of 3′-O-tert-butyldithiomethyl-dTTP (FIG. 17)

### 3′-O-methylthiomethyl-5′-O-tert-butyldimethylsilyl thymidine (2a)

To a stirring solution of the 5′-O-tert-butyldimethylsilyl thymidine (1a, 1.07 g, 3 mmol) in DMSO (10 mL) was added acetic acid (2.6 mL, 45 mmol) and acetic anhydride (8.6 mL, 90 mmol). The reaction mixture was stirred overnight at room temperature. Then the mixture was added slowly to a saturated solution of sodium bicarbonate under vigorous stirring and extracted with ethyl acetate (3×30 mL). The combined organic layers were dried over Na₂SO₄ and filtered. The filtrate was concentrated to dryness under reduced pressure and the compound was purified by silica gel column chromatography (ethyl acetate/hexane: 1:2) to give pure product 2a (0.97 g, 74%). 1H NMR (400 MHz, CDCl₃) δ: 8.16 (s, 1H), 7.48 (s, 1H), 6.28 (m, 1H), 4.62 (m, 2H), 4.46 (m, 1H), 4.10 (m, 1H), 3.78-3.90 (m, 2H), 2.39 (m, 1H), 2.14 (s, 3H), 1.97 (m, 1H), 1.92 (s, 3H), 0.93 (s, 9H), 0.13 (s, 3H); HRMS (FAB+) calc'd for C₁₈H₃₃N₂O₅SSi [(M+H)+]: 417.1879, found: 417.1890.

### 3′-O-tert-butyldithiomethyl-5′-O-tert-butyldimethylsilyl thymidine (3a)

3′-O-methylthiomethyl-5′-O-tert-butyldimethylsilyl thymidine (2a, 420 mg, 1 mmol) was dissolved in anhydrous dichloromethane (20 mL), followed by addition of triethylamine (0.18 mL, 1.31 mmol, 1.2 eq.) and molecular sieve (3 A, 2 g). The mixture was cooled in an ice bath after stirring at room temperature for 30 min and then a solution of sulfuryl chloride (redistilled, 0.1 mL, 1.31 mmol, 1.2 eq.) in anhydrous dichloromethane (3 mL) was added dropwise over 2 minutes. The ice bath was removed and the reaction mixture was stirred further for 30 min. Then potassium p-toluenethiosulfonate (375 mg, 1.65 mmol) in anhydrous DMF (2 mL) was added to the mixture. Stirring was continued at room temperature for an additional hour followed by addition of tert-butyl mercaptan (1 mL). The reaction mixture was stirred at room temperature for 30 min and quickly filtered through celite. The filter was washed with dichloromethane and the organic fraction was concentrated to give crude product 3a.

### 3′-O-tert-butyldithiomethyl-thymidine (4a)

Without isolation, the crude compound 3a was dissolved in THF (10 mL) and a THF solution of tetrabutylammonium fluoride (1.0M, 1.04 mL, 1.04 mmol) was added. The reaction mixture was stirred at room temperature for 4 hours. The reaction mixture was concentrated in vacuo, saturated NaHCO₃ solution (50 mL) was added and the mixture was extracted with dichloromethane (3×20 mL). The organic layer was dried over anhydrous Na₂SO₄, filtered, concentrated and the obtained crude mixture was purified by flash column chromatography (dichloromethane/methanol: 20:1) to give 3′-O-tert-butyldithiomethyl-thymidine 4a (132 mg, 35% from compound 2a). ¹H NMR (300 MHz, CDCl₃) δ: 7.41 (q, J=1.2 Hz, 1H), 6.15 (dd, J=7.4, 6.5 Hz, 1H), 4.89-4.82 (m, 2H), 4.62-4.54 (m, 1H), 4.15 (q, J=3.0 Hz, 1H), 3.97-3.86 (m, 2H), 2.42 (ddd, J=7.5, 4.8, 2.5 Hz, 2H), 1.95 (d, J=1.2 Hz, 3H), 1.36 (s, 8H).

### 3′-O-tert-butyldithiomethyl-dTTP (5a)

3′-O-tert-butyldithiomethyl-thymidine (4a, 50 mg, 0.13 mmol), tetrabutylammonium pyrophosphate (197 mg, 0.36 mmol) and 2-chloro-4-H-1,3,2-benzodioxaphosphorin-4-one (44 mg, 0.22 mmol) were dried separately overnight under high vacuum at ambient temperature. The tetrabutylammonium pyrophosphate was dissolved in dimethylformamide (DMF, 1 mL) under argon followed by addition of tributylamine (1 mL). This mixture was injected into the solution of 2-chloro-4-H-1,3,2-benzodioxaphosphorin-4-one in (DMF, 2 mL) under argon. After stirring for 1 h, the reaction mixture was added to the solution of 3′-O-tert-butyldithiomethyl-thymidine and stirred further for 1 hour at room temperature. Iodine solution (0.02 M iodine/pyridine/water) was then injected into the reaction mixture until a permanent brown color was observed. After 10 min, water (30 mL) was added and the reaction mixture was stirred at room temperature for an additional 2 hours. The resulting solution was extracted with ethyl acetate (2×30 mL). The aqueous layer was concentrated under vacuum and the residue was diluted with 5 ml of water. The crude mixture was then purified with anion exchange chromatography on DEAE-Sephadex A-25 at 4° C. using a gradient of TEAB (pH 8.0; 0.1-1.0 M). The crude product was further purified by reverse-phase HPLC to afford 5a, which was characterized by MALDI-TOF MS: calc'd for C₁₅H₂₇N₂O₁₄P₃S₂: 616.4, found: 615.4.

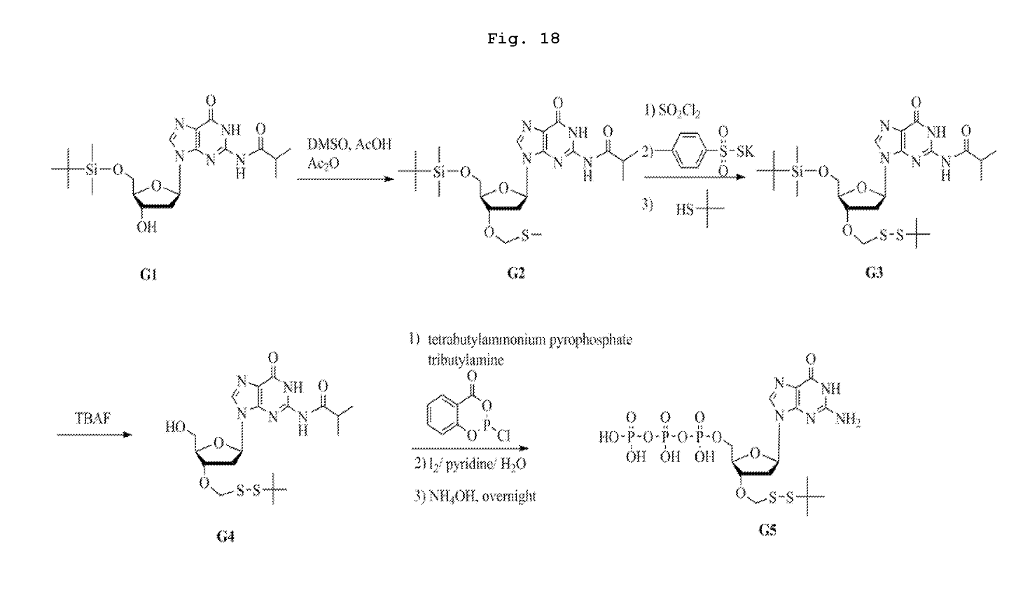

### Synthesis of 3′-O-tert-butyldithiomethyl-dGTP (FIG. 18)

### N²-isobutyryl-3′-O-methylthiomethyl-5′-O-tert-butyldimethylsilyl-2′-deoxyguanosine (G2)

To a stirring solution of N²-isobutyryl-5′-O-tert-butyldimethylsilyl-2′-deoxyguanosine (G1, 1.31 g, 3 mmol) in DMSO (10 mL) was added acetic acid (2.6 mL, 45 mmol) and acetic anhydride (8.6 mL, 90 mmol). The reaction mixture was stirred at room temperature until the reaction was complete, which was monitored by TLC. Then the mixture was added slowly to a saturated solution of sodium bicarbonate under vigorous stirring and extracted with ethyl acetate (3×30 mL). The combined organic layers were dried over Na₂SO₄ and filtered. The filtrate was concentrated to dryness under reduced pressure and the compound was purified by silica gel column chromatography (DCM/methanol: 20:1) to give pure product G2 (75%, 1.15 g). ¹H NMR (400 MHz, CDCl₃) δ 12.10 (d, J=2.9 Hz, 1H), 9.17 (d, J=3.0 Hz, 1H), 8.03 (m, 1H), 6.18 (td, J=6.9, 2.9 Hz, 1H), 4.74-4.60 (m, 3H), 4.13 (dq, J=6.8, 3.3 Hz, 1H), 3.84-3.75 (m, 2H), 2.78 (m, 1H), 2.54 (m, 2H), 2.16 (s, 3H), 1.33-1.22 (m, 6H), 0.96-0.87 (m, 9H), 0.09 (dd, J=6.7, 3.8 Hz, 6H).

### N²-isobutyryl-3′-O-tert-butyldithiomethyl-5′-O-tert-butyldimethylsilyl-2′-deoxyguanosine (G3)

N²-isobutyryl-3′-O-methylthiomethyl-5′-O-tert-butyldimethylsilyl-2′-deoxyguanosine (G2, 511 mg, 1.0 mmol) was dissolved in anhydrous dichloromethane (20 mL), followed by addition of triethylamine (0.17 mL, 1.2 mmol) and molecular sieve (3 A, 2 g). The mixture was cooled in an ice-bath after stirring at room temperature for 30 min and then a solution of sulfuryl chloride (0.095 mL, 1.2 mmol) in anhydrous dichloromethane (3 mL) was added dropwise over 2 minutes. The ice-bath was removed and the reaction mixture was stirred further for 30 min. Then potassium 4-toluenethiosulfonate (341 mg, 1.5 mmol) in anhydrous DMF (2 mL) was added to the mixture. Stirring was continued at room temperature for an additional hour followed by addition of tert-butyl mercaptan (1 mL). The reaction mixture was stirred at room temperature for 30 min and quickly filtered through celite. The filter was washed with dichloromethane and the organic fraction was concentrated to give crude product G3.

### N²-isobutyryl-3′-O-tert-butyldithiomethyl-2′-deoxyguanosine (G4)

Without isolation, the crude compound G3 was dissolved in THF (10 mL) and a THF solution of tetrabutylammonium fluoride (1.0M, 1.04 mL, 1.04 mmol) was added. The reaction mixture was stirred at room temperature for 4 hours. The reaction mixture was concentrated in vacuo, saturated NaHCO₃ solution (50 mL) was added and the mixture was extracted with dichloromethane (3×20 mL). The organic layer was dried over anhydrous Na₂SO₄, filtered, concentrated and the obtained crude mixture was purified by flash column chromatography (dichloromethane/methanol: 20:1) to give N²-isobutyryl-3′-O-tert-butyldithiomethyl-2′-deoxyguanosine G4 (155 mg, 33% from compound G2). ¹H NMR (400 MHz, CDCl₃) δ 12.19 (s, 1H), 9.44 (s, 1H), 7.97 (s, 1H), 6.17 (dd, J=8.4, 5.9 Hz, 1H), 5.04 (s, 1H), 4.92-4.80 (m, 2H), 4.76-4.64 (m, 1H), 4.26 (q, J=2.6 Hz, 1H), 3.98 (dd, J=12.2, 2.8 Hz, 1H), 3.80 (d, J=12.3 Hz, 1H), 2.91-2.73 (m, 2H), 2.49 (m, 1H), 1.35 (s, 9H), 1.36-1.22 (m, 6H). ¹³C NMR (75 MHz, CDCl₃) δ 179.60, 155.80, 148.10, 147.96, 139.11, 122.30, 86.29, 81.22, 78.96, 63.21, 48.07, 38.18, 36.64, 30.29, 19.39, 19.34.

### 3′-O-tert-butyldithiomethyl-dGTP (G5)

N²-isobutyryl-3′-O-tert-butyldithiomethyl-2′-deoxyguanosine (G4, 50 mg, 0.11 mmol), tetrabutylammonium pyrophosphate (180 mg, 0.33 mmol) and 2-chloro-4-H-1,3,2-benzodioxaphosphorin-4-one (44 mg, 0.22 mmol) were dried separately overnight under high vacuum at ambient temperature. The tetrabutylammonium pyrophosphate was dissolved in dimethylformamide (DMF, 1 mL) under argon followed by addition of tributylamine (1 mL). This mixture was injected into the solution of 2-chloro-4-H-1,3,2-benzodioxaphosphorin-4-one in (DMF, 2 mL) under argon. After stirring for 1 h, the reaction mixture was added to the solution of N²-isobutyryl-3′-O-tert-butyldithiomethyl-2′-deoxyguanosine and stirred further for 1 hour at room temperature. Iodine solution (0.02 M iodine/pyridine/water) was then injected into the reaction mixture until a permanent brown color was observed. After 10 min, water (30 mL) was added and the reaction mixture was stirred at room temperature for an additional 2 hours. The resulting solution was extracted with ethyl acetate. The aqueous layer was concentrated in vacuo to approximately 20 mL, then concentrated NH₄OH (20 ml) was added and the mixture stirred overnight at room temperature. The resulting mixture was concentrated under vacuum and the residue was diluted with 5 ml of water. The crude mixture was then purified with anion exchange chromatography on DEAE-Sephadex A-25 at 4° C. using a gradient of TEAB (pH 8.0; 0.1-1.0 M). The crude product was further purified by reverse-phase HPLC to afford G5. HRMS (ESI) calc'd for C₁₅H₂₅N₅O₁₃P₃S₂ [(M−H)⁻]640.0103, found: 640.0148.

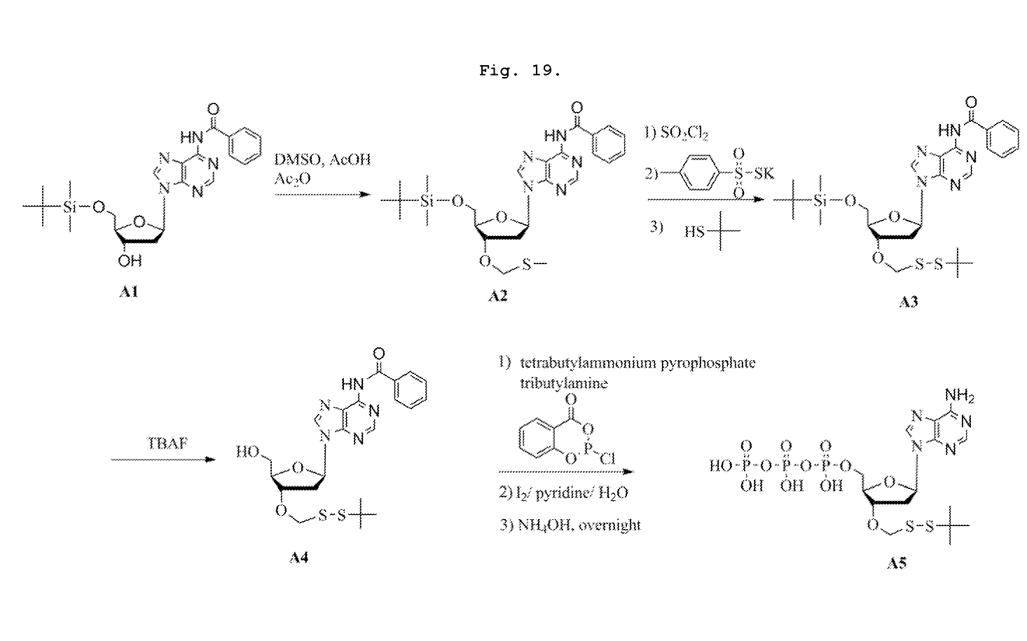

### Synthesis of 3′-O-tert-butyldithiomethyl-dATP (FIG. 19)

N⁶-Benzoyl-5′-O-tert-butyldimethylsilyl-3′-O-methylthiomethyl-2′-deoxyadenosine (A2). To a stirring solution of the N⁶-Benzoyl-5′-O-tert-butyldimethylsilyl-2′-deoxyadenosine (A1, 1.41 g, 3 mmol) in DMSO (10 mL) was added acetic acid (3 mL) and acetic anhydride (9 mL). The reaction mixture was stirred at room temperature until the reaction was complete, which was monitored by TLC. Then the mixture was added slowly to a solution of sodium bicarbonate under vigorous stirring and extracted with ethyl acetate (3×30 mL). The combined organic layers were dried over Na₂SO₄ and filtered. The filtrate was concentrated to dryness under reduced pressure and the residue of the desired compound was purified by silica gel column chromatography (dichloromethane/methanol: 30:1) to give pure product A2 (1.39 g, 88%). ¹H NMR (400 MHz, CDCl₃) δ 9.12 (s, 1H), 8.81 (s, 1H), 8.35 (s, 1H), 8.10-8.01 (m, 2H), 7.68 (m, 1H), 7.49 (m, 2H), 6.53 (dd, J=7.5, 6.0 Hz, 1H), 4.78-4.65 (m, 3H), 4.24 (dt, J=4.3, 3.1 Hz, 1H), 3.98-3.81 (m, 2H), 2.80-2.60 (m, 2H), 2.21 (s, 3H), 0.94 (s, 10H), 0.13 (s, 6H); MS (APCI⁺) calc'd for C₂₆H₃₆N₄O₄SSi: 528.74, found: 529.4 [M+H]⁺.

### N⁶—Benzoyl-5′-O-tert-butyldimethylsilyl-3′-O-tert-butyldithiomethyl-2′-deoxyadenosine (A3)

N⁶—Benzoyl-5′-O-tert-butyldimethylsilyl-3′-O-methylthiomethyl-2′-deoxyadenosine (A2, 529 mg, 1.0 mmol) was dissolved in anhydrous dichloromethane (20 mL), followed by addition of triethylamine (0.17 mL, 1.2 mmol) and molecular sieve (3 Å, 2 g). The mixture was cooled in an ice bath after stirring at room temperature for 30 min and then a solution of sulfuryl chloride (0.095 mL, 1.2 mmol) in anhydrous dichloromethane (3 mL) was added dropwise over 2 minutes. The ice bath was removed and the reaction mixture was stirred further for 30 min. Then potassium 4-toluenethiosulfonate (341 mg, 1.5 mmol) in anhydrous DMF (2 mL) was added to the mixture. Stirring was continued at room temperature for an additional hour followed by addition of tert-butyl mercaptan (1 mL). The reaction mixture was stirred at room temperature for 30 min and quickly filtered through celite. The filter was washed with dichloromethane and the organic fraction was concentrated to give crude product A3.

### N⁶-Benzoyl-3′-O-tert-butyldithiomethyl-2′-deoxyadenosine (A4)

Without isolation, the crude compound A3 was dissolved in THF (10 mL) and a THF solution of tetrabutylammonium fluoride (1.0M, 1.04 mL, 1.04 mmol) was added. The reaction mixture was stirred at room temperature for 4 hours. The reaction mixture was concentrated in vacuo, saturated NaHCO₃ solution (50 mL) was added and the mixture was extracted with dichloromethane (3×20 mL). The organic layer was dried over anhydrous Na₂SO₄, filtered, concentrated and the obtained crude mixture was purified by flash column chromatography (dichloromethane/methanol: 20:1) to give N⁶-Benzoyl-3′-O-tert-butyldithiomethyl-2′-deoxyadenosine A4 (128 mg, 26% from compound A2). ¹H NMR (400 MHz, DMSO-d₆) δ 11.18 (s, 1H), 8.77 (s, 1H), 8.71 (s, 1H), 8.10-8.02 (m, 2H), 7.66 (t, J=7.6 Hz, 1H), 7.56 (t, J=7.6 Hz 2H), 6.47 (dd, J=8.0, 6.0 Hz, 1H), 5.15 (t, J=5.5 Hz, 1H), 5.00 (s, 2H), 4.65 (dt, J=5.4, 2.4 Hz, 1H), 4.12 (td, J=4.7, 2.2 Hz, 1H), 3.02-2.88 (m, 1H), 2.84 (q, J=7.3 Hz, 2H), 2.61 (m, 1H), 1.35 (s, 9H).

### 3′-O-tert-butyldithiomethyl-dATP (A5)

N⁶—Benzoyl-3′-O-tert-butyldithiomethyl-2′-deoxyadenosine (A4, 50 mg, 0.10 mmol), tetrabutylammonium pyrophosphate (180 mg, 0.33 mmol) and 2-chloro-4-H-1,3,2-benzodioxaphosphorin-4-one (44 mg, 0.22 mmol) were dried separately overnight under high vacuum at ambient temperature. The tetrabutylammonium pyrophosphate was dissolved in dimethylformamide (DMF, 1 mL) under argon followed by addition of tributylamine (1 mL). This mixture was injected into the solution of 2-chloro-4-H-1,3,2-benzodioxaphosphorin-4-one in (DMF, 2 mL) under argon. After stirring for 1 h, the reaction mixture was added to the solution of N⁶-Benzoyl-3′-O-tert-butyldithiomethyl-2′-deoxyadenosine and stirred further for 1 hour at room temperature. Iodine solution (0.02 M iodine/pyridine/water) was then injected into the reaction mixture until a permanent brown color was observed. After 10 min, water (30 mL) was added and the reaction mixture was stirred at room temperature for additional 2 hours. The resulting solution was extracted with ethyl acetate. The aqueous layer was concentrated in vacuo to approximately 20 mL, then concentrated NH₄OH (20 ml) was added and stirring continued overnight at room temperature. The resulting mixture was concentrated under vacuum and the residue was diluted with 5 ml of water. The crude mixture was then purified by anion exchange chromatography on DEAE-Sephadex A-25 at 4° C. using a gradient of TEAB (pH 8.0; 0.1-1.0 M). The crude product was further purified by reverse-phase HPLC to afford A5, which was characterized by MALDI-TOF MS calc'd for C₁₅H₂₆N₅O₁₂P₃S₂: 625.4, found: 625.0.

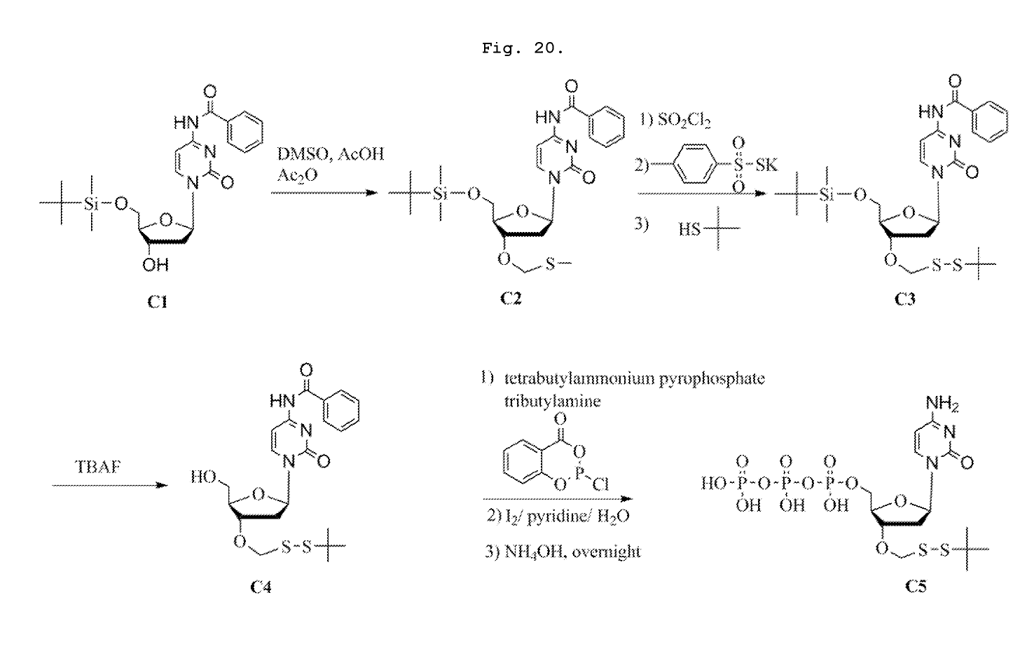

### Synthesis of 3′-O-tert-butyldithiomethyl-dCTP (FIG. 20)

### N⁴-Benzoyl-3′-O-methylthiomethyl-5′-O-tert-butyldimethylsilyl-2′-deoxycytidine (C2)

To a stirring solution of N⁴-Benzoyl-5′-O-tert-butyldimethylsilyl-2′-deoxycytidine (C1, 1.5 g, 3.4 mmol) in DMSO (6.5 mL) was added acetic acid (2.91 mL) and acetic anhydride (9.29 mL). The reaction mixture was stirred at room temperature for 2 days. Then the reaction mixture was added dropwise to solution of sodium bicarbonate and extracted by ethyl acetate (50 ml×3). The obtained crude product was purified by column chromatography (ethyl acetate/hexane: 8:2) to give pure product C2 (1.26 g, 74%) as a white solid. 1H NMR (400 MHz, CDCl₃) 8.43 (d, J=7.4 Hz, 1H), 7.92 (d, J=7.6 Hz, 2H), 7.69-7.50 (m, 4H), 6.31 (t, J=6.1 Hz, 1H), 4.75-4.59 (m, 2H), 4.51 (dt, J=6.2, 3.9 Hz, 1H), 4.20 (dt, J=3.7, 2.6 Hz, 1H), 4.01 (dd, J=11.4, 2.9 Hz, 1H), 3.86 (dd, J=11.4, 2.4 Hz, 1H), 2.72 (ddd, J=13.8, 6.2, 4.1 Hz, 1H), 2.18 (s, 4H), 0.97 (s, 9H), 0.17 (d, J=3.9 Hz, 6H). HRMS (ESI⁺) calc'd for C₂₄H₃₅N₃O₅SSi [(M+H)⁺]: 506.2145, found: 506.2146.

### N⁴-Benzoyl-3′-O-tert-butyldithiomethyl-5′-O-tert-butyldimethylsilyl-2′-deoxycytidine (C3)

N⁴—Benzoyl-3′-O-methylthiomethyl-5′-O-tert-butyldimethylsilyl-2′-deoxycytidine (C2, 1.01 g, 2 mmol) was dissolved in anhydrous dichloromethane (8 mL), followed by addition of triethylamine (278 μL, 2 mmol) and molecular sieves (3 Å, 1 g). The mixture was cooled in an ice bath after stirring at room temperature for 0.5 hour and then a solution of sulfuryl chloride (161 μL, 2.2 mmol) in anhydrous dichloromethane (8 mL) was added dropwise. The ice bath was removed and the reaction mixture was stirred further for 0.5 hour. Then potassium p-toluenethiosulfonate (678 mg, 3 mmol) in anhydrous DMF (1 mL) was added to the mixture. Stirring was continued at room temperature for an additional 1 hour followed by addition of tert-butyl mercaptan (1 mL). The reaction mixture was stirred at room temperature for 0.5 hour and quickly filtered. The solvent was removed under reduced pressure and the residue was dissolved in ethyl acetate and washed in brine (3×50 mL). The combined organic layers were dried over Na₂SO₄ and filtered. The filtrate was concentrated to dryness under reduced pressure and the residue of the desired compound was purified by silica gel column chromatography using a gradient of ethyl acetate-hexane from 3:7 (v/v) to 5:5 (v/v), yielding 959 mg (83%) C3 as a white foam. ¹H NMR (400 MHz, CDCl₃) δ 8.43 (d, J=7.4 Hz, 1H), 7.92 (d, J=7.6 Hz, 2H), 7.69-7.50 (m, 4H), 6.31 (t, J=6.1 Hz, 1H), 4.75-4.59 (m, 2H), 4.51 (dt, J=6.2, 3.9 Hz, 1H), 4.20 (dt, J=3.7, 2.6 Hz, 1H), 4.01 (dd, J=11.4, 2.9 Hz, 1H), 3.86 (dd, J=11.4, 2.4 Hz, 1H), 2.72 (ddd, J=13.8, 6.2, 4.1 Hz, 1H), 2.18 (s, 4H), 0.97 (s, 9H), 0.17 (d, J=3.9 Hz, 6H), 0.10 (s, 2H). HRMS (ESI⁺) calc'd for: C₂₇H₄₁N₃O₅S₂Si [(M+Na)⁺]: 602.2155, found: 602.2147.

### N⁴-Benzoyl-3′-O-tert-butyldithiomethyl-2′-deoxycytidine (C4)

To a stirred solution of N⁴-Benzoyl-3′-O-tert-butyldithiomethyl-5′-O-tert-butyldimethylsilyl-2′-deoxycytidine (C3, 958 mg, 1.66 mmol) in a mixture of tetrahydrofuran (24 ml), tetrabutylammonium fluoride (1.0M, 2.48 mL) was added in small portions, and stirred at room temperature for 3 hours. The reaction mixture was poured into a saturated sodium bicarbonate solution (50 mL) and extracted with ethyl acetate (3×50 mL). The combined organic layers were dried over Na₂SO₄ and filtered. The filtrate was concentrated to dryness under reduced pressure and the residue of the desired compound was purified by silica gel column chromatography using a gradient of ethyl acetate-hexane from 5:5 (v/v), affording 435 mg (56%) C4 as a solid white powder. 1H NMR (400 MHz, Methanol-d₄) δ 8.52 (d, J=7.5 Hz, 1H), 8.04-7.96 (m, 2H), 7.71-7.60 (m, 2H), 7.61-7.51 (m, 2H), 6.28-6.19 (m, 1H), 4.95-4.86 (m, 2H), 4.54 (dt, J=6.0, 3.0 Hz, 1H), 4.23 (q, J=3.4 Hz, 1H), 3.92-3.76 (m, 2H), 2.70 (ddd, J=13.9, 6.0, 2.9 Hz, 1H), 2.25 (ddd, J=13.6, 7.2, 6.2 Hz, 1H), 1.37 (s, 9H). HRMS (ESI⁺) calc'd for C₂₁H₂₇N₃O₅S₂ [(M+Na)⁺]: 488.1290, found: 488.1297.

### 3′-O-tert-butyldithiomethyl-dCTP (C5)

N⁴—Benzoyl-3′-O-tert-butyldithiomethyl-2′-deoxycytidine (C4, 50 mg, 0.11 mmol), tetrabutylammonium pyrophosphate (180 mg, 0.33 mmol) and 2-chloro-4-H-1,3,2-benzodioxaphosphorin-4-one (44 mg, 0.22 mmol) were dried separately overnight under high vacuum at ambient temperature. The tetrabutylammonium pyrophosphate was dissolved in dimethylformamide (DMF, 1 mL) under argon followed by addition of tributylamine (1 mL). This mixture was injected into the solution of 2-chloro-4-H-1,3,2-benzodioxaphosphorin-4-one in (DMF, 2 mL) under argon. After stirring for 1 h, the reaction mixture was added to the solution of N⁴-benzoyl-3′-O-tert-butyldithiomethyl-2′-deoxycytidine and stirred further for 1 hour at room temperature. Iodine solution (0.02 M iodine/pyridine/water) was then injected into the reaction mixture until a permanent brown color was observed. After 10 min, water (30 mL) was added and the reaction mixture was stirred at room temperature for an additional 2 hours. The resulting solution was extracted with ethyl acetate. The aqueous layer was concentrated in vacuo to approximately 20 mL, then concentrated NH₄OH (20 ml) was added and the mixture stirred overnight at room temperature. The resulting mixture was concentrated under vacuum and the residue was diluted with 5 ml of water. The crude mixture was then purified by anion exchange chromatography on DEAE-Sephadex A-25 at 4° C. using a gradient of TEAB (pH 8.0; 0.1-1.0 M). The crude product was further purified by reverse-phase HPLC to afford C5. HRMS (ESI−) calc'd for C₁₄H₂₅N₃O₁₃P₃S₂[(M−H)⁻]: 600.0042, found: 600.0033.

### Synthesis of 3′-O-ethyldithiomethyl-2′-deoxynucleoside-5′-triphosphates (3′-O-DTM-dNTPs, FIG. 6)

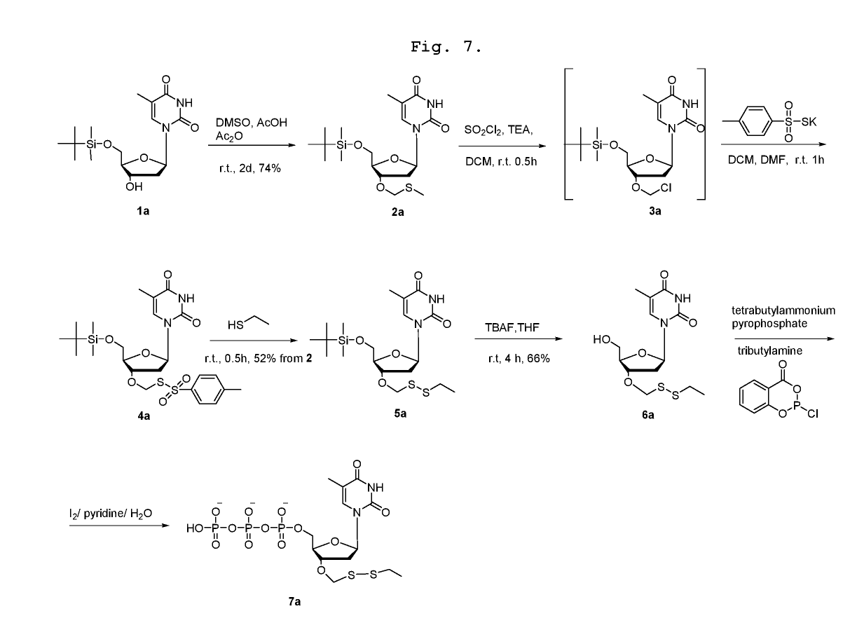

### Synthesis of 3′-O-ethyldithiomethyl-dTTP (7a) (FIG. 7)

### 3′-O-methylthiomethyl-5′-O-tert-butyldimethylsilyl thymidine (2a)

To a stirring solution of the 5′-O-tert-butyldimethylsilyl thymidine (1a, 1.07 g, 3 mmol) in DMSO (10 mL) was added acetic acid (2.6 mL, 45 mmol) and acetic anhydride (8.6 mL, 90 mmol). The reaction mixture was stirred at room temperature until the reaction was complete (48 h), which was monitored by TLC. Then the mixture was added slowly to a saturated solution of sodium bicarbonate under vigorous stirring and extracted with ethyl acetate (3×30 mL). The combined organic layers were dried over Na₂SO₄ and filtered. The filtrate was concentrated to dryness under reduced pressure and the compound was purified by silica gel column chromatography (ethyl acetate/hexane: 1:2) to give pure product 2a (0.97 g, 74%). 1H NMR (400 MHz, CDCl₃) δ: 8.16 (s, 1H), 7.48 (s, 1H), 6.28 (m, 1H), 4.62 (m, 2H), 4.46 (m, 1H), 4.10 (m, 1H), 3.78-3.90 (m, 2H), 2.39 (m, 1H), 2.14 (s, 3H), 1.97 (m, 1H), 1.92 (s, 3H), 0.93 (s, 9H), 0.13 (s, 3H); HRMS (FAB⁺) calc'd for C₁₈H₃₃N₂O₅SSi [(M+H)+]: 417.1879, found: 417.1890.

### 3′-O-ethyldithiomethyl-5′-O-tert-butyldimethylsilyl thymidine (5a)

3′-O-methylthiomethyl-5′-O-tert-butyldimethylsilyl thymidine (2a, 453 mg, 1.09 mmol) was dissolved in anhydrous dichloromethane (20 mL), followed by addition of triethylamine (0.18 mL, 1.31 mmol, 1.2 eq.) and molecular sieve (3 Å, 2 g). The mixture was cooled in an ice-bath after stirring at room temperature for 30 min and then a solution of sulfuryl chloride (redistilled, 0.1 mL, 1.31 mmol, 1.2 eq.) in anhydrous dichloromethane (3 mL) was added dropwise over 2 minutes. The ice-bath was removed and the reaction mixture was stirred further for 30 min. Then potassium p-toluenethiosulfonate (375 mg, 1.65 mmol, 1.5 eq.) in anhydrous DMF (2 mL) was added to the mixture. Stirring was continued at room temperature for additional hour followed by addition of ethanethiol (0.17 mL, 2.2 mmol, 2 eq.). The reaction mixture was stirred at room temperature for 30 min and quickly filtered through celite. The filter was washed with dichloromethane and the organic fraction was concentrated. The residue was purified by Flash column chromatography (ethyl acetate/hexane: 2:1) to give pure product 5a (261 mg, 52%). ¹H NMR (400 MHz, CDCl₃) δ: 8.66 (br. s, 1H), 7.49 (s, 1H), 6.30 (dd, J=7.2, 11.2 Hz, 1H), 4.83 (dd, J=15.2, 37.2 Hz, 2H), 4.49 (d, J=8.0 Hz, 1H), 4.14 (d, J=3.2 Hz, 1H), 3.80 (m, 2H), 2.77 (dd, J=10.0, 19.6 Hz, 2H), 2.47 (m, 1H), 2.03 (m, 1H), 1.93 (s, 3H), 1.35 (t, J=8.8 Hz, 2H), 0.95 (s, 9H), 0.14 (s, 6H). ¹³C NMR (75 MHz, CDCl₃): δ 164.00, 150.59, 135.61, 111.35, 85.33, 79.76, 77.98, 77.81, 63.89, 38.10, 33.64, 26.33, 18.74, 14.84, 12.89, −4.85, −5.03.

### 3′-O-ethyldithiomethyl thymidine (3′-O-DTM-T, 6a)

3′-O-ethyldithiomethyl-5′-O-tert-butyldimethylsilyl thymidine (5a, 240 mg, 0.52 mmol) was dissolved in anhydrous THF (10 mL) and a THF solution of tetrabutylammonium fluoride (1.0 M, 1.04 mL, 1.04 mmol, 1.5 eq.) was added. The reaction mixture was stirred at room temperature for 4 hours. The reaction mixture was concentrated in vacuo, saturated NaHCO₃ solution (50 mL) was added and the mixture was extracted with dichloromethane (3×20 mL). The organic layer was dried over anhydrous Na₂SO₄, filtered, concentrated and the obtained crude mixture was purified by flash column chromatography (dichloromethane/methanol: 20/1) to give 3′-O-ethyldithiomethyl thymidine 6a (119 mg, 66%). ¹H NMR (300 MHz, CDCl₃) δ: 7.44 (s, 1H), 6.15 (t, J=8.8 Hz, 1H), 4.83 (dd, J=11.4, 23.4 Hz, 2H), 4.46 (m, 1H), 4.12 (m, 2H), 3.80 (m, 2H), 2.77 (dd, J=7.5, 14.7 Hz, 2H), 2.34 (m, 2H), 2.04 (s, 1H), 1.90 (s, 3H), 1.34 (t, J=7.5 Hz, 3H). ¹³C NMR (75 MHz, CDCl₃): δ 164.37, 150.88, 137.26, 111.53, 87.20, 85.29, 78.52, 62.82, 37.49, 33.59, 14.85, 12.89. HRMS (ESI⁺) calc'd for C₁₃H₂₀N₂O₅S₂Na [(M+Na)⁺]: 371.0711, found: 371.0716.

### 3′-O-ethyldithiomethyl-dTTP (3′-O-DTM-TTP 7a)

3′-O-ethyldithiomethyl thymidine (6a, 50 mg, 0.14 mmol), tetrabutylammonium pyrophosphate (197 mg, 0.36 mmol, 2.5 eq.) and 2-chloro-4-H-1,3,2-benzodioxaphosphorin-4-one (44 mg, 0.22 mmol, 1.5 eq) were dried separately overnight under high vacuum at ambient temperature. The tetrabutylammonium pyrophosphate was dissolved in dimethylformamide (DMF, 1 mL) under argon followed by addition of tributylamine (1 mL). This mixture was injected into the solution of 2-chloro-4-H-1,3,2-benzodioxaphosphorin-4-one in (DMF, 2 mL) under argon. After stirring for 1 h, the reaction mixture was added to the solution of 3′-O-ethyldithiomethyl thymidine and stirred further for 1 hour at room temperature. Iodine solution (0.02 M iodine/pyridine/water) was then injected into the reaction mixture until a permanent brown color was observed. After 10 min, water (30 mL) was added and the reaction mixture was stirred at room temperature for additional 2 hours. The resulting solution was extracted with ethyl acetate (2×30 mL). The aqueous layer was concentrated in vacuo to approximately 20 mL, and transferred to two centrifuge tubes (50 mL). Brine (1.5 mL) and absolute ethanol (35 mL) were added to each tube, followed by vigorous shaking. After being placed at −80° C. for 2 h, the tube was centrifuged (10 min at 4200 rpm) to afford the crude product as a white precipitate. The supernatant was poured out, the white precipitate was diluted with 5 ml of water and purified by ion exchange chromatography on DEAE-Sephadex® A-25 at 4° C. using a gradient of TEAB (pH 8.0; 0.1-1.0 M). The crude product was further purified by reverse-phase HPLC to afford 7a. HRMS (ESI calc'd for C₁₃H₂₂N₂O₁₄S₂P₃ [(M−H)⁻]: 586.9725, found: 586.9727. ³¹P-NMR (121.4 MHz, D₂0): 6-10.83 (s, 1P), −10.98 (s, 1P), −20.53 (t, J=21 Hz, 1P).

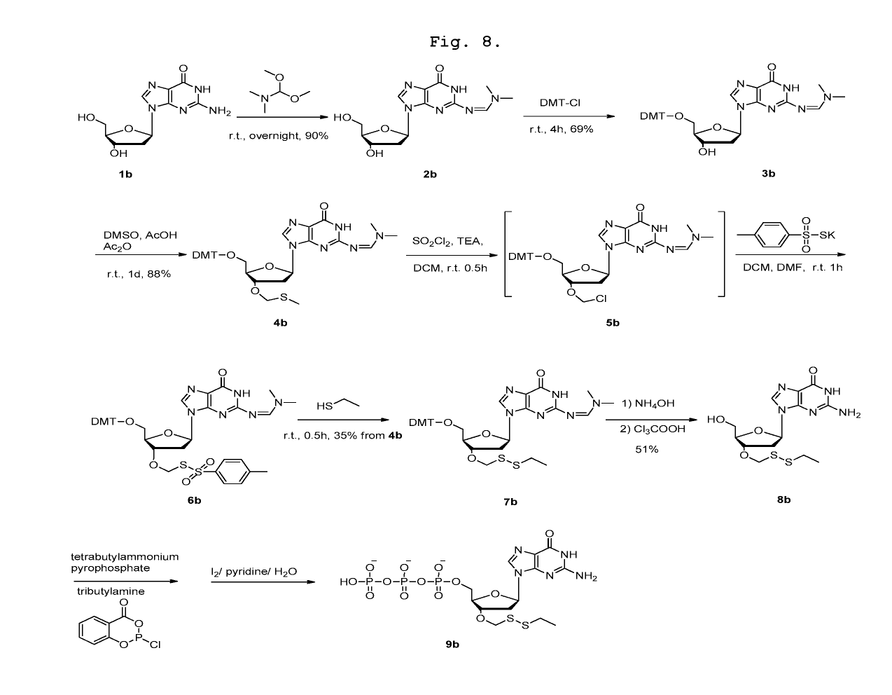

### Synthesis of 3′-O-ethyldithiomethyl-dGTP (9b) (FIG. 8)

### N²-Dimethylformamidino-2′-deoxyguanosine (2b)

To a suspension of 2′-deoxyguanosine (1b, 1.33 g, 5 mmol) in dry DMF (20 mL) was added N, N-dimethylformamide dimethyl acetal (1.5 mL, 11 mmol) and the reaction mixture was stirred at room temperature overnight. The solvent was removed and the residue triturated with methanol and filtered. The solid was washed with methanol to give a white solid 2b (90%, 1.44 g). ¹H NMR (400 MHz, DMSO-d₆) δ 11.28 (s, 1H), 8.57 (s, 1H), 8.04 (s, 1H), 6.26 (dd, J=7.9, 6.1 Hz, 1H), 5.30 (d, J=3.8 Hz, 1H), 4.93 (t, J=5.5 Hz, 1H), 4.40 (dt, J=5.8, 2.8 Hz, 1H), 3.85 (td, J=4.5, 2.5 Hz, 1H), 3.56 (m, 2H), 3.17 (s, 3H), 3.04 (s, 3H), 2.60 (m, 1H), 2.25 (m, 1H).

### N²-Dimethylformamidino-5′-O-DMT-2′-deoxyguanosine (3b)

N²-DMF-2′-deoxyguanosine (2b, 1.38 g, 4.3 mmol, 1 eq.) was dissolved in anhydrous pyridine (30 mL), and 4, 4′-dimethoxytrityl chloride (1.74 g, 5.2 mmol, 1.2 eq.) was added. After stirring at room temperature for 4 hours, the reaction mixture was poured into saturated sodium bicarbonate solution (200 mL) and the precipitate was collected by suction filtration, washed with water and hexane. The obtained crude produce was purified by silica gel column chromatography (dichloromethane/methanol: 30:1) to give N²-DMF-5′-O-DMT-2′-deoxyguanosine 3b (1.84 g, 69%) as a white solid. ¹H NMR (400 MHz, CDCl₃) δ 9.13 (s, 1H), 8.57 (s, 1H), 7.71 (s, 1H), 7.3 (m, 2H), 7.34-7.20 (m, 6H), 7.18 (t, J=2.8 Hz, 1H), 6.90-6.72 (m, 4H), 6.40 (t, J=6.6 Hz, 1H), 4.64 (m, 1H), 4.15 (m, 1H), 3.81 (m, 1H), 3.78 (m, 6H), 3.43 (dd, J=10.1, 4.8 Hz, 1H), 3.32 (dd, J=10.1, 5.0 Hz, 1H), 3.11 (s, 3H), 3.06 (s, 3H), 2.65-2.48 (m, 2H).

### N²-Dimethylformamidino-3′-O-methylthiomethyl-5′-O-DMT-2′-deoxyguanosine (4b)

To a stirred solution of the N²-DMF-5′-O-DMT-2′-deoxyguanosine (1.33 g, 2.1 mmol) in DMSO (10 mL) was added acetic acid (2.1 mL, 36 mmol) and acetic anhydride (5.4 mL, 56 mmol). The reaction mixture was stirred at room temperature until the reaction was complete (24 h), which was monitored by TLC. Then the mixture was added slowly to a solution of sodium bicarbonate under vigorous stirring and extracted with ethyl acetate (3×30 mL). The combined organic layers were dried over Na₂SO₄ and filtered. The filtrate was concentrated to dryness under reduced pressure and the desired compound was purified by silica gel column chromatography (ethyl acetate/hexane: 1:2) to give pure product 4b (1.27 g, 88%) as a white solid. ¹H NMR (400 MHz, CDCl₃) δ 9.73 (s, 1H), 8.58 (s, 1H), 7.73 (s, 1H), 7.47-7.38 (m, 2H), 7.37-7.17 (m, 7H), 6.87-6.77 (m, 4H), 6.33 (dd, J=7.7, 6.1 Hz, 1H), 4.72-4.63 (m, 3H), 4.25-4.18 (m, 1H), 3.80 (s, 6H), 3.34 (m, 2H), 3.14 (s, 3H), 3.09 (s, 3H), 2.64-2.48 (m, 2H), 2.13 (s, 3H); ¹³C NMR (75 MHz, CDCl₃): δ 158.96, 158.69, 158.50, 150.61, 144.88, 136.19, 136.02, 130.41, 128.49, 128.33, 127.35, 120.85, 113.61, 86.96, 84.19, 83.64, 74.01, 64.05, 55.65, 41.74, 38.31, 35.61, 14.26.

### N²-Dimethylformamidino-3′-O-ethyldithiomethyl-5′-O-DMT-2′-deoxyguanosine (7b)

N²-DMF-3′-O-methylthiomethyl-5′-O-DMT-2′-deoxyguanosine (684 mg, 1.0 mmol) was dissolved in anhydrous dichloromethane (20 mL), followed by addition of triethylamine (0.17 mL, 1.2 mmol, 1.2 eq.) and molecular sieve (3 Å, 2 g). The mixture was cooled in an ice-bath after stirring at room temperature for 30 min and then a solution of sulfuryl chloride (0.095 mL, 1.2 mmol, 1.2 eq.) in anhydrous dichloromethane (3 mL) was added dropwise over 2 minutes. The ice-bath was removed and the reaction mixture was stirred further for 30 min. Then potassium 4-toluenethiosulfonate (341 mg, 1.5 mmol, 1.5 eq.) in anhydrous DMF (2 mL) was added to the mixture. Stirring was continued at room temperature for an additional hour followed by addition of ethanethiol (0.16 mL, 2.0 mmol, 2 eq.). The reaction mixture was stirred at room temperature for 30 min and quickly filtered through celite. The filter was washed with dichloromethane and the organic fraction was concentrated. The residue was purified by silica gel column chromatography (ethyl acetate/hexane: 2:1) to give pure product 7b (255 mg, 35%). ¹H NMR (400 MHz, CDCl₃) δ 9.55 (s, 1H), 8.58 (s, 1H), 7.73 (s, 1H), 7.47-7.38 (m, 2H), 7.37-7.27 (m, 6H), 7.27-7.18 (m, 1H), 6.88-6.79 (m, 4H), 6.34 (t, J=7.0 Hz, 1H), 4.86 (s, 2H), 4.65 (m, 1H), 4.25 (m, 1H), 3.80 (d, J=0.9 Hz, 6H), 3.44-3.28 (m, 2H), 3.16-3.07 (s, 3H), 3.10 (s, 3H), 2.75 (qd, J=7.4, 0.7 Hz, 2H), 2.62-2.54 (m, 2H), 1.29 (t, J=13.5, 4H). ¹³C NMR (75 MHz, CDCl₃): δ 158.99, 158.50, 157.30, 150.57, 144.84, 136.06, 135.95, 130.41, 128.47, 128.36, 127.38, 120.88, 113.65, 87.04, 84.12, 83.61, 79.68, 78.48, 64.02, 55.65, 41.74, 38.34, 35.60, 33.60, 14.87, 14.59

### 3′-O-ethyldithiomethyl-2′-deoxyguanosine (8b)

The mixture of N²-DMF-3′-ethyldithiomethyl-5′-O-DMT-2′-deoxyguanosine (280 mg, 0.38 mmol), ammonium hydroxide (10 mL) and methanol (10 mL) was stirred at room temperature until the reaction was complete (4 h), which was monitored by TLC. After evaporation of the solvent under reduced pressure, the crude solid was treated with 3% trichloroacetic acid solution in dichloromethane for 10 min. Then the mixture was added slowly to the solution of sodium bicarbonate under vigorous stirring and extracted with ethyl acetate (3×30 mL). The combined organic layers were dried over Na₂SO₄ and filtered. The filtrate was concentrated to dryness under reduced pressure and the desired compound was purified by silica gel column chromatography (dichloromethane/methanol: 20/1) to give 3′-ethyldithiomethyl-2′-deoxyguanosine 8b (72 mg, 51%). ¹H NMR (300 MHz, DMSO-d₆) δ 10.61 (s, 1H), 7.93 (s, 1H), 6.45 (bs, 2H), 6.07 (dd, J=8.5, 5.7 Hz, 1H), 5.06 (bs, 1H), 4.95 (s, 2H), 4.51 (d, J=5.3 Hz, 1H), 3.99 (m, 1H), 3.55 (d, J=4.3 Hz, 2H), 2.80 (q, J=7.3 Hz, 2H), 2.72-2.56 (m, 1H), 2.43-2.39 (m, 1H), 1.28 (t, J=7.3 Hz, 3H). HRMS (ESI⁺) calc'd for C₁₃H₁₉N₅O₄S₂Na [(M+Na)⁺]: 396.0776, found: 396.0770.

### 3′-O-ethyldithiomethyl-dGTP (9b)

The preparation procedure was similar to the synthesis of 7a. 3′-ethyldithiomethyl-2′-deoxyguanosine (8b, 64 mg, 0.17 mmol), tetrabutylammonium pyrophosphate (238 mg, 0.44 mmol, 2.5 eq.) and 2-chloro-4-H-1,3,2-benzodioxaphosphorin-4-one (53 mg, 0.27 mmol, 1.5 eq) were dried separately over night under high vacuum at ambient temperature in three round bottom flasks. The tetrabutylammonium pyrophosphate was dissolved in dimethylformamide (DMF, 1 mL) under argon followed by addition of tributylamine (1 mL). The mixture was injected into the solution of 2-chloro-4-H-1,3,2-benzodioxaphosphorin-4-one in (DMF, 2 mL) under argon. After stirring for 1 h, the reaction mixture was added to the solution of 3′-O-ethyldithiomethyl thymidine and stirred further for 1 hour at room temperature. Iodine solution (0.02 M iodine/pyridine/water) was then injected into the reaction mixture until a permanent brown color was observed. After 10 min, water (30 mL) was added and the reaction mixture was stirred at room temperature for an additional 2 hours. The resulting solution was extracted with ethyl acetate (2×30 mL). The aqueous layer was concentrated in vacuo to approximately 20 mL, and transferred to two centrifuge tubes (50 mL). Brine (1.5 mL) and absolute ethanol (35 mL) were added to each tube, followed by vigorous shaking. After being placed at −80° C. for 2 h, the tube was centrifuged (10 min at 4200 rpm) to offer the crude product as a white precipitate. The supernatant was poured out, the white precipitate was diluted with 5 ml of water and purified with anion exchange chromatography on DEAE-Sephadex® A-25 at 4° C. using a gradient of TEAB (pH 8.0; 0.1-1.0 M). The crude product was further purified by reverse-phase HPLC to afford 9b.

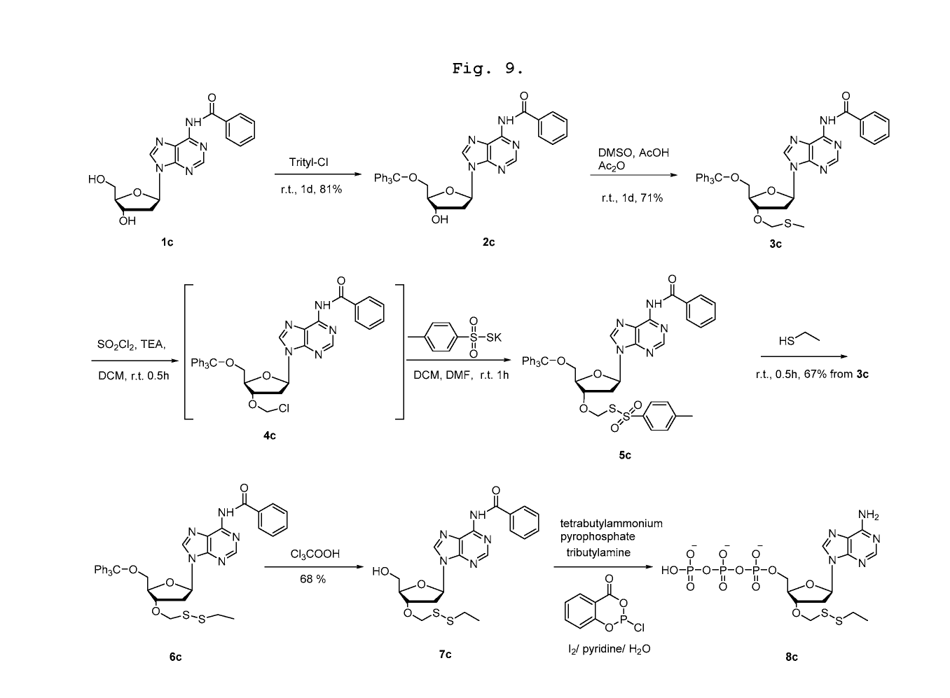

### Synthesis of 3′-O-ethyldithiomethyl-dATP (8c) (FIG. 9)

### N⁶-Benzoyl-5′-O-trityl-2′-deoxyadenosine (2c)

N⁶—Benzoyl-2′-deoxyadenosine (1c, 1.07 g, 3.0 mmol, 1 eq.) was dissolved in anhydrous pyridine (30 mL), and trityl chloride (1.00 g, 3.6 mmol, 1.2 eq.) was added. After stirring at room temperature for 1 day, the reaction mixture was poured into saturated sodium bicarbonate solution (200 mL) and the precipitate was collected by suction filtration, washed with water and hexane. The obtained crude product was purified by silica gel column chromatography (dichloromethane/methanol: 30:1) to give N⁶-Benzoyl-5′-O-trityl-2′-deoxygadenosine 2c (1.45 g, 81%) as a white solid. ¹H NMR (400 MHz, CDCl₃) δ 9.12 (s, 1H), 8.74 (s, 1H), 8.15 (s, 1H), 8.08-8.00 (m, 2H), 7.62 (m, 1H), 7.52 (m, 2H), 7.46-7.38 (m, 6H), 7.34-7.20 (m, 9H), 6.50 (t, J=6.5 Hz, 1H), 4.74 (d, J=4.7 Hz, 1H), 4.19 (td, J=4.8, 3.5 Hz, 1H), 3.49-3.42 (m, 2H), 2.90 (m, 1H), 2.58 (m, 1H).

### N⁶-Benzoyl-3′-O-methylthiomethyl-5′-O-trityl-2′-deoxyadenosine (3c)

To a stirred solution of the N⁶-Benzoyl-5′-O-trityl-2′-deoxyadenosine (1.72 g, 2.93 mmol) in DMSO (10 mL) was added acetic acid (2.8 mL, 48 mmol) and acetic anhydride (72 mL, 75 mmol). The reaction mixture was stirred at room temperature until the reaction was complete (24 h), which was monitored by TLC. Then the mixture was added slowly to a solution of sodium bicarbonate under vigorous stirring and extracted with ethyl acetate (3×30 mL). The combined organic layers were dried over Na₂SO₄ and filtered. The filtrate was concentrated to dryness under reduced pressure and the desired compound was purified by silica gel column chromatography (ethyl acetate/hexane: 1:2) to give pure product 3c (1.35 g, 71%) as a white solid. ¹H NMR (400 MHz, CDCl₃) δ 9.07 (s, 1H), 8.74 (s, 1H), 8.19 (s, 1H), 8.05 (dt, J=7.2, 1.4 Hz, 2H), 7.67-7.49 (m, 3H), 7.49-7.39 (m, 6H), 7.36-7.22 (m, 9H), 6.48 (dd, J=7.6, 6.0 Hz, 1H), 4.79 (m, 1H), 4.66 (m, 2H), 4.31 (td, J=4.8, 2.7 Hz, 1H), 3.51-3.38 (m, 2H), 2.89 (m, 1H), 2.64 (m, 1H), 2.15 (s, 3H). ¹³C NMR (75 MHz, CDCl₃): δ 165.03, 153.03, 151.82, 149.88, 143.87, 141.78, 134.05, 133.19, 129.27, 129.02, 128.35, 128.28, 127.67, 123.83, 87.52, 85.43, 85.59, 76.85, 74.05, 63.98, 37.94, 30.13, 14.27.

### N⁶-Benzoyl-3′-O-ethylthyldithiomethyl-5′-O-trityl-2′-deoxyadenosine (6c)

3′-O-methylthiomethyl-5′-O-Trityl-2′-deoxyadenosine (3c, 861 mg, 1.31 mmol) was dissolved in anhydrous dichloromethane (20 mL), followed by addition of triethylamine (0.19 mL, 1.5 mmol, 1.2 eq.) and molecular sieve (3 Å, 2 g). The mixture was cooled in an ice-bath after stirring at room temperature for 0.5 hour and then a solution of sulfuryl chloride (0.11 mL, 1.5 mmol, 1.2 eq.) in anhydrous dichloromethane (3 mL) was added dropwise during 2 minutes. The ice-bath was removed and the reaction mixture was stirred further for 30 min. Then potassium p-toluenethiosulfonate (595 mg, 2.62 mmol, 1.5 eq.) in anhydrous DMF (3 mL) was added to the mixture. Stirring was continued at room temperature for an additional hour followed by addition of ethanethiol (0.47 mL, 6.55 mmol, 2 eq.). The reaction mixture was stirred at room temperature for 30 min and quickly filtered through celite. The filter was washed with dichloromethane and the organic fraction was concentrated. The residue was purified by silica gel column chromatography (ethyl acetate/hexane: 2:1) to give pure product 6c (615 mg, 67%). ¹H NMR (400 MHz, CDCl₃) δ 9.04 (s, 1H), 8.74 (s, 1H), 8.18 (s, 1H), 8.05 (d, J=7.2 Hz, 2H), 7.67-7.59 (m, 1H), 7.59-7.50 (m, 2H), 7.50-7.38 (m, 6H), 7.36-7.21 (m, 9H), 6.47 (dd, J=7.8, 5.9 Hz, 1H), 4.90 (s, 2H), 4.75 (dt, J=5.4, 2.5 Hz, 1H), 4.35 (td, J=4.9, 2.5 Hz, 1H), 3.45 (m, 2H), 3.00-2.86 (m, 1H), 2.85-2.71 (m, 2H), 2.68 (m, 1H), 1.33 (t, J=7.4, 3H).

### N⁶-Benzoyl-3′-O-ethyldithiomethyl-2′-deoxyadenosine (7c)

N⁶-Benzoyl-3′-ethyldithiomethyl-5′-O-trityl-2′-deoxyadenosine (6c), 381 mg, 0.54 mmol) was treated with 3% trichloroacetic acid solution in dichloromethane at room temperature for 10 min. Then the mixture was added slowly to a solution of sodium bicarbonate under vigorous stirring and extracted with ethyl acetate (3×30 mL). The combined organic layers were dried over Na₂SO₄ and filtered. The filtrate was concentrated to dryness under reduced pressure and the residue of the desired compound was purified by silica gel column chromatography (dichloromethane/methanol: 20/1) to give 7c (169 mg, 68%). ¹H NMR (400 MHz, DMSO-d₆) δ 11.18 (s, 1H), 8.77 (s, 1H), 8.71 (s, 1H), 8.10-8.02 (m, 2H), 7.66 (t, J=7.6 Hz, 1H), 7.56 (t, J=7.6 Hz 2H), 6.47 (dd, J=8.0, 6.0 Hz, 1H), 5.15 (t, J=5.5 Hz, 1H), 5.00 (s, 2H), 4.65 (dt, J=5.4, 2.4 Hz, 1H), 4.12 (td, J=4.7, 2.2 Hz, 1H), 3.72-3.55 (m, 2H), 3.02-2.88 (m, 1H), 2.84 (q, J=7.3 Hz, 2H), 2.61 (m, 1H), 1.40-1.15 (m, 3H). ¹³C NMR (75 MHz, DMSO-d₆): δ 166.47, 152.83, 152.47, 151.27, 143.87, 134.22, 133.30, 129.33, 126.78, 86.18, 84.79, 79.35, 78.80, 62.37, 36.93, 33.04, 15.21.

### 3′-O-ethyldithiomethyl-dATP (8c)

Compound 7c (100 mg, 0.22 mmol) and proton sponge (60 mg, 0.28 mmol) were dried in a vacuum desiccator over P₂O₅ overnight and dissolved in trimethyl phosphate (2 ml). Freshly distillated POCl₃ (30 μL, 0.32 mmol) was added dropwise and the mixture was stirred for 2 h at 0° C. Tributylammonium pyrophosphate (452 mg, 0.82 mmol) and tributylamine (450 μL, 1.90 mmol) in anhydrous DMF (1.9 mL) was added in one portion at room temperature and the solution stirred for additional 30 min. Triethylammonium bicarbonate solution (TEAB, 0.1 M; pH 8.0; 10 mL) was added and the mixture was stirred for 1 h at room temperature. Then concentrated NH₄OH (10 mL) was added and stirring continued for 3 h at room temperature. The mixture was concentrated under vacuum and the crude product was purified by anion exchange chromatography on DEAE-Sephadex® A-25 at 4° C. using a gradient of TEAB (pH 8.0; 0.1-1.0 M), followed by a further purification by reverse-phase HPLC to afford 8c.

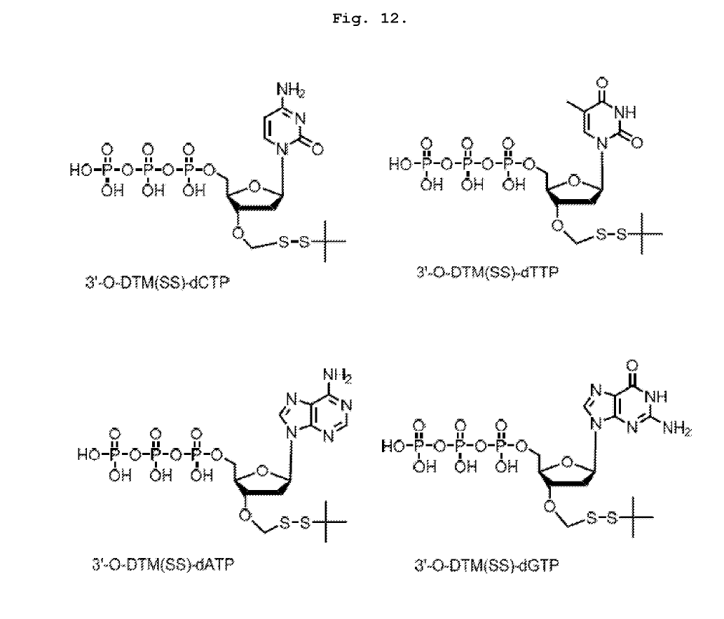

### Synthesis of 3′-O-ethyldithiomethyl-dCTP (7d) (FIG. 12)

### N⁴-Benzoyl-3′-O-methylthiomethyl-5′-O-tert-butyldimethylsilyl-2′-deoxycytidine (2d)

To a stirred solution of N⁴-Benzoyl-5′-O-tert-butyldimethylsilyl-2′-deoxycytidine (1.5 g, 3.37 mmol) in any DMSO (6.5 ml) was added acetic acid (2.9 ml) and acetic anhydride (9.3 ml). The mixture was stirred at room temperature for 2 days, and then quenched by adding saturated NaHCO₃ solution (50 ml). The reaction mixture was extracted with ethyl acetate (50 mL×3) and the combined organic layers dried over anhydrous Na₂SO₄. The crude product after concentration was purified by flash column chromatography (ethyl acetate/hexane: 8:2) to give a white powder (1.26 g, 74%). ¹H NMR (400 MHz, Methanol-d₄) δ 8.50 (d, J=7.5 Hz, 1H), 8.05-7.97 (m, 2H), 7.72-7.61 (m, 2H), 7.61-7.52 (m, 2H), 6.23 (t, J=6.3 Hz, 1H), 4.81-4.71 (m, 2H), 4.58 (dt, J=6.4, 3.3 Hz, 1H), 4.24 (q, J=3.1 Hz, 1H), 4.02 (dd, J=11.5, 3.3 Hz, 1H), 3.91 (dd, J=11.5, 2.8 Hz, 1H), 2.75-2.59 (m, 1H), 2.24 (dt, J=13.9, 6.3 Hz, 1H), 2.18 (s, 3H), 0.98 (s, 9H), 0.19 (d, J=3.3 Hz, 6H). HRMS (APCI⁺) calc'd for C₂₄H₃₅N₃O₅SSi [(M+H)⁺]: 506.2145, found: 506.2124.

### N⁴-Benzoyl-3′-O-ethyldithiomethyl-5′-O-tert-butyldimethylsilyl-2′-deoxycytidine (5d)

To a stirred solution of 2d (612 mg, 1.21 mmol) in anhydrous dichloromethane (10 ml), triethylamine (168 μL, 1.21 mmol) and 4 A molecular sieve (1 g) were added. The reaction mixture was stirred at room temperature for 30 minutes and then cooled in an ice-bath. SO₂Cl₂ (98 μL, 1.21 mmol) dissolved in anhydrous dichloromethane (5 ml) was added dropwise to the mixture. Then the ice bath was removed, and the reaction mixture was stirred for at room temperature for 30 minutes. Potassium p-toluenethiosulfonate (425 mg, 1.9 mmol) dissolved in anhydrous DMF (625 μL) was added into the reaction mixture, and after being stirred for additional 30 minutes, ethanethiol (174 μL, 2.4 mmol) was added and stirring continued at room temperature for an additional 30 minutes. The reaction mixture was filtered, concentrated, and then extracted with saturated sodium bicarbonate and dichloromethane (3×50 mL). The organic phase was dried over Na₂SO₄, concentrated, and purified by flash column chromatography using a gradient of ethyl acetate-hexane from 5:5 (v/v) to 8:2 (v/v), yielding 563.2 mg (84%) white foam. ¹H NMR (400 MHz, Methanol-d₄) δ 8.55-8.42 (m, 1H), 8.00 (dt, J=8.4, 1.1 Hz, 2H), 7.70-7.45 (m, 4H), 6.23 (q, J=6.9, 6.4 Hz, 1H), 5.01-4.88 (m, 2H), 4.56 (tt, J=6.5, 3.1 Hz, 1H), 4.30-4.19 (m, 1H), 4.00 (m, J=11.4, 3.2, 0.8 Hz, 1H), 3.94-3.76 (m, 1H), 2.81 (qd, J=7.3, 0.9 Hz, 2H), 2.76-2.68 (m, 1H), 2.31-2.17 (m, 1H), 1.40-1.25 (m, 3H), 1.00-0.85 (m, 9H), 0.21-0.03 (m, 6H). HRMS (APCI⁺) calc'd for C₂₅H₃₇N₃O₅S₂Si [(M+Na)^{+]}: 574.1841, found: 574.1826.

### N⁴—Benzoyl-3′-O-ethyldithiomethyl-2′-deoxycytidine (6d)

To a stirred solution of 5d (526 mg, 0.95 mmol) in a mixture of tetrahydrofuran (3 ml) and methanol (9 ml), NH₄F (1.8 g) powder was added in small portions and stirred at room temperature for 3 days. The crude product was concentrated and purified by flash column chromatography using a gradient of ethyl acetate-hexane from 2:8 (v/v) to 7:3 (v/v), affording a white solid powder (233 mg, 56%). ¹H NMR (400 MHz, Methanol-d₄) 1H NMR (400 MHz, Methanol-d4) δ 8.54 (d, J=7.5 Hz, 1H), 8.04-7.97 (m, 2H), 7.71-7.43 (m, 4H), 6.25 (t, 1H), 5.01-4.89 (m, 2H), 4.56 (dt, J=6.0, 3.0 Hz, 1H), 4.23 (q, J=3.4 Hz, 1H), 3.92-3.76 (m, 2H), 2.84 (q, J=7.3 Hz, 2H), 2.71 (m, J=13.9, 5.9, 2.9 Hz, 1H), 2.31-2.19 (m, 1H), 1.36 (t, J=7.3 Hz, 3H). HRMS (APCI⁺) calc'd for C₁₉H₂₃N₃O₅S₂[(M+H)⁺]: 438.1157, found: 438.1136.

### 3′-O-ethyldithiomethyl-dCTP (7d)

Compound 6d (60 mg, 0.14 mmol) and proton sponge (40 mg, 0.19 mmol) were dried in a vacuum desiccator over P₂O₅ overnight, dissolved in trimethyl phosphate (1 ml) and cooled in an ice-bath. Freshly distillated POCl₃ (19 μL, 0.2 mmol) was added dropwise and stirred for 2 h at 0° C. Tributylammonium pyrophosphate (255 mg, 0.47 mmol) and tributylamine (27.6 μL, 0.12 mmol) in anhydrous DMF (1.5 mL) was added in one portion at room temperature followed by an additional stirring for 30 min. Triethylammonium bicarbonate solution (TEAB) (0.1 M; pH 8.0; 7.5 mL) was added and the mixture was stirred for 1 h at room temperature. Then concentrated NH₄OH (7.5 mL) was added and stirring continued overnight at room temperature. The mixture was concentrated under vacuum and the crude product was purified by anion exchange chromatography on DEAE-Sephadex® A-25 at 4° C. using a gradient of TEAB (pH 8.0; 0.1-1.0 M), followed by a further purification by reverse-phase HPLC to afford 7d.

### REFERENCES

Anderson, E. P. et al. (2008) A system for multiplexed direct electrical detection of DNA synthesis. Sens Actuators B Chem 129:78-86.
Bentley, D. R. et al. (2008) Accurate whole human genome sequencing using reversible terminator chemistry. Nature 456:53-59.
Bowers, J. et al. (2009) Virtual terminator nucleotides for next-generation DNA sequencing. Nature Methods, 6:593-595.
Branton, D. et al. (2008) The potential and challenges of nanopore sequencing. Nat Biotechnol 26:1146-1153.
Edwards, J. R. et al. (2001) DNA sequencing using biotinylated dideoxynucleotides and mass spectrometry. Nucleic Acids Res, 29:E104.
Eid, J. et al. (2008) Real-time DNA sequencing from single polymerase molecule. Science 323:133-138.
Fuller, C. W. et al. (2009) The challenges of sequencing by synthesis. Nat Biotechnol 27:1013-1023.
Gerstein, A. S., ed. Molecular Biology Problem Solver: A Laboratory Guide, Ch. 10, “Nucleotides, Oligonucleotides, and Polynucleotides” (2001).
Guo, J. et al. (2008) Four-color DNA sequencing with 3′-O-modified nucleotide reversible terminators and chemically cleavable fluorescent dideoxynucleotides. Proc Natl Acad Sci USA 105:9145-9150.
Haghighi, F. et al. (2008) Genetic architecture of the human tryptophan hydroxylase 2 gene: existence of neural isoforms and relevance for major depression. Mol Psychiatry. 13:813-820.
Harris, T. D. et al. (2008) Single-molecule DNA sequencing of a viral genome. Science, 320:106-109.
Hawkins, R. D. et al. (2010) Next-generation genomics: an integrative approach. Nat. Rev. Genet. 11:476-486.
Hutter D. et al. (2010) Labeled nucleoside triphosphates with reversibly terminating aminoalkoxy groups. Nucleosides Nucleotides & Nucleic Acids 29:879-895.
Ju, J. et al. (1995) Energy transfer fluorescent dye-labeled primers for DNA sequencing and analysis. Proc Natl Acad Sci USA 92:4347-4351.
Ju, J. et al. (2003) Massive parallel method for decoding DNA and RNA. U.S. Pat. No. 6,664,079.
Ju, J. et al. (2006) Four-color DNA sequencing by synthesis using cleavable fluorescent nucleotide reversible terminators. Proc Natl Acad Sci USA 103:19635-19640.
Knapp D. C. et al. (2011) Fluoride-Cleavable, Fluorescently Labelled Reversible Terminators: Synthesis and Use in Primer Extension. Chem. Eur. J., 17, 2903-2915.
Kwiatkowski M. (2007) Compounds for protecting hydroxyls and methods for their use. U.S. Pat. No. 7,279,563, granted Oct. 9, 2007.
Landgraf, P. et al. (2007) A mammalian microRNA expression atlas based on small library RNA sequencing. Cell 129:1401-1414.
Li, Z. et al. (2003) A Photocleavable Fluorescent Nucleotide for DNA Sequencing and Analysis. Proc Natl Acad Sci USA 100:414-419.
Marti, A. A. et al. (2007) Design and characterization of two-dye and three-dye binary fluorescent probes for mRNA detection. Tetrahedron 63:3591-3600.
McKernan, K. J. et al. (2009) Sequence and structural variation in a human genome uncovered by short-read, massively parallel ligation sequencing using two base encoding. Genome Research 19:1527-1541.
Metzker M. L. et al. (1994) Termination of DNA synthesis by novel 3′-modified-deoxyribonucleoside 5′-triphosphates. Nucleic Acids Res. 22, 4259-4267.
Morozova, O. et al. (2009) Applications of new sequencing technologies for transcriptome analysis. Annu Rev Genomics Hum Genet 10:135-51.
Muller S. et al. (2011) Method for producing trinucleotides. PCT International Patent Application Publication No. WO 2011/061114, published May 26, 2011.
Ng, S. B. et al. (2010) Massively parallel sequencing and rare disease. Hum Mol Genet 19:R19-R24.
Park, P. J. (2009) ChIP-seq: advantages and challenges of a maturing technology. Nat Rev Genet 10:669-680.
Pelletier H. et al. (1994) Structures of ternary complexes of rat DNA polymerase beta, a DNA template-primer, and ddCTP. Science 264:1891-1903.
Ronaghi, M et al. (1998) A sequencing method based on real-time pyrophosphate. Science, 281:364-365.
Ronaghi, M. (2001) Pyrosequencing sheds light on DNA sequencing. Genome Res., 11:3-11.
Rothberg, J. M. et al. (2011) An integrated semiconductor device enabling non-optical genome sequencing. Nature 475:348-352.
Ruparel, H. et al. (2004) Digital detection of genetic mutations using SPC-sequencing. Genome Res. 14:296-300.
Ruparel, H. et al. (2005) Design and Synthesis of a 3′-O-allyl Photocleavable Fluorescent Nucleotide as a Reversible Terminator for DNA Sequencing By Synthesis. Proc Natl Acad Sci USA 102:5932-5937.
Sanger, F. et al. (1977) DNA sequencing with chain-terminating inhibitors. Proc Natl Acad Sci USA 74:5463-5467.
Semenyuk A. (2006) Synthesis of RNA using 2′-O-DTM protection. JACS, 128, 12356-12357.
Seo, T. S et al. (2005) Four-Color DNA Sequencing by Synthesis on Chip Using Photocleavable Fluorescent Nucleotide Analogues. Proc Natl Acad Sci USA 102:5926-5931.
Shen, Y. et al. (2010) A SNP discovery method to assess variant allele probability from next-generation resequencing data. Genome Res 20:273-280.
Smith, L. M. et al. (1986) Fluorescence detection in automated DNA sequencing analysis. Nature 321:674-679.
Strug, L. J. et al. (2009) Centrotemporal sharp wave EEG trait in rolandic epilepsy maps to Elongator Protein Complex 4 (ELP4) Eur J Human Genet 17:1171-1181.
Welch M. B. et al. (1999) Synthesis of fluorescent, photolabile 3′-O-protected nucleoside triphosphates for the base addition sequencing scheme. Nucleosides Nucleotides 18, 197-201.
Wheeler, D. A. et al. (2008) The complete genome of an individual by massively parallel DNA sequencing. Nature 452:872-877.
Wu, J. et al. (2007) 3′-O-modified Nucleotides as Reversible Terminators for Pyrosequencing. Proc Natl Acad Sci USA 104:16462-16467.

What is claimed:

1. A method for determining the identity of a nucleotide residue of a single-stranded DNA in a solution comprising:
(a) contacting the single-stranded DNA, having a primer hybridized to a portion thereof, with a DNA polymerase and a deoxyribonucleotide triphosphate (dNTP) analogue under conditions permitting the DNA polymerase to catalyze incorporation of the dNTP analogue into the primer if it is complementary to the nucleotide residue of the single-stranded DNA which is immediately 5′ to a nucleotide residue of the single-stranded DNA hybridized to the 3′ terminal nucleotide residue of the primer, so as to form a DNA extension product, wherein (1) the dNTP analogue has the structure: 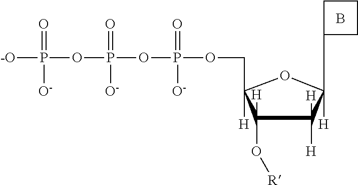
wherein B is a base and is adenine, guanine, cytosine, or thymine, and (2) R′ is (i) —CH₂N₃ or 2-nitrobenzyl, (ii) is a hydrocarbyl, or a substituted hydrocarbyl, having a mass of less than 300 daltons, or (iii) is an dithiol moiety; and
(b) determining whether incorporation of the dNTP analogue into the primer to form a DNA extension product has occurred in step (a) by determining if an increase in hydrogen ion concentration of the solution has occurred, wherein (i) if the dNTP analogue has been incorporated into the primer, determining from the identity of the incorporated dNTP analogue the identity of the nucleotide residue in the single-stranded DNA complementary thereto, thereby determining the identity of the nucleotide residue in the single-stranded DNA, and (ii) if no change in hydrogen ion concentration has occurred, iteratively performing step (a), wherein in each iteration of step (a) the dNTP analogue comprises a base which is a different type of base from the type of base of the dNTP analogues in every preceding iteration of step (a), until a dNTP analogue is incorporated into the primer to form a DNA extension product, and determining from the identity of the incorporated dNTP analogue the identity of the nucleotide residue in the single-stranded DNA complementary thereto, thereby determining the identity of the nucleotide residue in the single-stranded DNA.

2. A method for determining the sequence of consecutive nucleotide residues in a single-stranded DNA in a solution comprising:
(a) contacting the single-stranded DNA, having a primer hybridized to a portion thereof, with a DNA polymerase and a deoxyribonucleotide triphosphate (dNTP) analogue under conditions permitting the DNA polymerase to catalyze incorporation of the dNTP analogue into the primer if it is complementary to the nucleotide residue of the single-stranded DNA which is immediately 5′ to a nucleotide residue of the single-stranded DNA hybridized to the 3′ terminal nucleotide residue of the primer, so as to form a DNA extension product, wherein (1) the dNTP analogue has the structure: 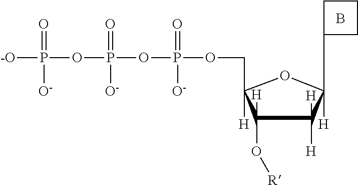
wherein B is a base and is adenine, guanine, cytosine, or thymine, and (2) R′ is (i) —CH₂N₃, or 2-nitrobenzyl, (ii) is a hydrocarbyl, or a substituted hydrocarbyl, having a mass of less than 300 daltons, or (iii) is an dithio moiety;
(b) determining whether incorporation of the dNTP analogue has occurred in step (a) by detecting an increase in hydrogen ion concentration of the solution, wherein an increase in hydrogen ion concentration indicates that the dNTP analogue has been incorporated into the primer to form a DNA extension product, and if so, determining from the identity of the incorporated dNTP analogue the identity of the nucleotide residue in the single-stranded DNA complementary thereto, thereby determining the identity of the nucleotide residue in the single-stranded DNA, and wherein no change in hydrogen ion concentration indicates that the dNTP analogue has not been incorporated into the primer in step (a);
(c) if no change in hydrogen ion concentration has been detected in step (b), iteratively performing steps (a) and (b), wherein in each iteration of step (a) for a given nucleotide residue, the identity of which is being determined, the dNTP analogue comprises a base which is a different type of base from the type of base of the dNTP analogues in every preceding iteration of step (a) for that nucleotide residue, until a dNTP analogue is incorporated into the primer to form a DNA extension product, and determining from the identity of the incorporated dNTP analogue the identity of the nucleotide residue in the single-stranded DNA complementary thereto, thereby determining the identity of the nucleotide residue in the single-stranded DNA;
(d) if an increase in hydrogen ion concentration has been detected and a dNTP analogue is incorporated, subsequently treating the incorporated dNTP nucleotide analogue so as to replace the R′ group thereof with an H atom thereby providing a 3′ OH group at the 3′ terminal of the DNA extension product; and
(e) iteratively performing steps (a) to (d), as necessary, for each nucleotide residue of the consecutive nucleotide residues of the single-stranded DNA to be sequenced, except that in each repeat of step (a) the dNTP analogue is (i) incorporated into the DNA extension product resulting from a preceding iteration of step (a) or step (c), and (ii) complementary to a nucleotide residue of the single-stranded DNA which is immediately 5′ to a nucleotide residue of the single-stranded DNA hybridized to the 3′ terminal nucleotide residue of the DNA extension product resulting from a preceding iteration of step (a) or step (c), so as to form a subsequent DNA extension product, with the proviso that for the last nucleotide residue to be sequenced step (d) is optional,
thereby determining the identity of each of the consecutive nucleotide residues of the single-stranded DNA so as to thereby determine the sequence of the consecutive nucleotide residues of the DNA.

3. A method for determining the identity of a nucleotide residue of a single-stranded RNA in a solution comprising:
(a) contacting the single-stranded RNA, having an RNA primer hybridized to a portion thereof, with a polymerase and a ribonucleotide triphosphate (rNTP) analogue under conditions permitting the polymerase to catalyze incorporation of the rNTP analogue into the RNA primer if it is complementary to the nucleotide residue of the single-stranded RNA which is immediately 5′ to a nucleotide residue of the single-stranded RNA hybridized to the 3′ terminal nucleotide residue of the RNA primer, so as to form an RNA extension product, wherein (1) the rNTP analogue has the structure: 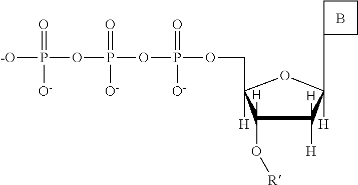
wherein B is a base and is adenine, guanine, cytosine, or uracil, and (2) R′ is (i) —CH₂N₃ or 2-nitrobenzyl, (ii) is a hydrocarbyl, or a substituted hydrocarbyl, having a mass of less than 300 daltons, or (iii) is an dithio moiety; and
(b) determining whether incorporation of the rNTP analogue into the RNA primer to form an RNA extension product has occurred in step (a) by determining if an increase in hydrogen ion concentration of the solution has occurred, wherein (i) if the rNTP analogue has been incorporated into the RNA primer, determining from the identity of the incorporated rNTP analogue the identity of the nucleotide residue in the single-stranded RNA complementary thereto, thereby determining the identity of the nucleotide residue in the single-stranded RNA, and (ii) if no change in hydrogen ion concentration has occurred, iteratively performing step (a), wherein in each iteration of step (a) the rNTP analogue comprises a base which is a different type of base from the type of base of the rNTP analogues in every preceding iteration of step (a), until an rNTP analogue is incorporated into the RNA primer to form an RNA extension product, and determining from the identity of the incorporated rNTP analogue the identity of the nucleotide residue in the single-stranded RNA complementary thereto, thereby determining the identity of the nucleotide residue in the single-stranded RNA.

4. A method for determining the sequence of consecutive nucleotide residues in a single-stranded RNA in a solution comprising:
(a) contacting the single-stranded RNA, having an RNA primer hybridized to a portion thereof, with a RNA polymerase and a ribonucleotide triphosphate (rNTP) analogue under conditions permitting the RNA polymerase to catalyze incorporation of the rNTP analogue into the RNA primer if it is complementary to the nucleotide residue of the single-stranded RNA which is immediately 5′ to a nucleotide residue of the single-stranded RNA hybridized to the 3′ terminal nucleotide residue of the RNA primer, so as to form an RNA extension product, wherein (1) the rNTP analogue has the structure: 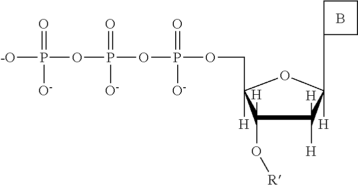
wherein B is a base and is adenine, guanine, cytosine, or uracil, and (2) R′ is (i) —CH₂N₃ or 2-nitrobenzyl, (ii) is a hydrocarbyl, or a substituted hydrocarbyl, having a mass of less than 300 daltons, or (iii) is an dithio moiety;
(b) determining whether incorporation of the rNTP analogue has occurred in step (a) by detecting an increase in hydrogen ion concentration of the solution, wherein an increase in hydrogen ion concentration indicates that the rNTP analogue has been incorporated into the RNA primer to form an RNA extension product, and if so, determining from the identity of the incorporated rNTP analogue the identity of the nucleotide residue in the single-stranded RNA complementary thereto, thereby determining the identity of the nucleotide residue in the single-stranded RNA, and wherein no change in hydrogen ion concentration indicates that the rNTP analogue has not been incorporated into the RNA primer in step (a);
(c) if no change in hydrogen ion concentration has been detected in step (b), iteratively performing steps (a) and (b), wherein in each iteration of step (a) for a given nucleotide residue, the identity of which is being determined, the rNTP analogue comprises a base which is a different type of base from the type of base of the rNTP analogues in every preceding iteration of step (a) for that nucleotide residue, until an rNTP analogue is incorporated into the RNA primer to form an RNA extension product, and determining from the identity of the incorporated rNTP analogue the identity of the nucleotide residue in the single-stranded RNA complementary thereto, thereby determining the identity of the nucleotide residue in the single-stranded RNA;
(d) if an increase in hydrogen ion concentration has been detected and an rNTP analogue is incorporated, subsequently treating the incorporated rNTP nucleotide analogue so as to replace the R′ group thereof with an H atom thereby providing a 3′ OH group at the 3′ terminal of the RNA extension product; and
(e) iteratively performing steps (a) to (d), as necessary, for each nucleotide residue of the consecutive nucleotide residues of the single-stranded RNA to be sequenced, except that in each repeat of step (a) the rNTP analogue is (i) incorporated into the RNA extension product resulting from a preceding iteration of step (a) or step (c), and (ii) complementary to a nucleotide residue of the single-stranded RNA which is immediately 5′ to a nucleotide residue of the single-stranded RNA hybridized to the 3′ terminal nucleotide residue of the RNA extension product resulting from a preceding iteration of step (a) or step (c), so as to form a subsequent RNA extension product, with the proviso that for the last nucleotide residue to be sequenced step (d) is optional,
thereby determining the identity of each of the consecutive nucleotide residues of the single-stranded RNA so as to thereby determine the sequence of the consecutive nucleotide residues of the RNA.

5. A method for determining the identity of a nucleotide residue of a single-stranded RNA in a solution comprising:
(a) contacting the single-stranded RNA, having a DNA primer hybridized to a portion thereof, with a reverse transcriptase and a deoxyribonucleotide triphosphate (dNTP) analogue under conditions permitting the reverse transcriptase to catalyze incorporation of the dNTP analogue into the DNA primer if it is complementary to the nucleotide residue of the single-stranded RNA which is immediately 5′ to a nucleotide residue of the single-stranded RNA hybridized to the 3′ terminal nucleotide residue of the DNA primer, so as to form a DNA extension product, wherein (1) the dNTP analogue has the structure: 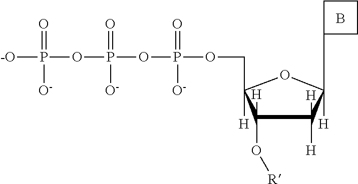
wherein B is a base and is adenine, guanine, cytosine, or thymine, and (2) R′ is (i) —CH₂N₃ or 2-nitrobenzyl, (ii) is a hydrocarbyl, or a substituted hydrocarbyl, having a mass of less than 300 daltons, or (iii) is an dithio moiety; and
(b) determining whether incorporation of the dNTP analogue into the DNA primer to form a DNA extension product has occurred in step (a) by determining if an increase in hydrogen ion concentration of the solution has occurred, wherein (i) if the dNTP analogue has been incorporated into the DNA primer, determining from the identity of the incorporated dNTP analogue the identity of the nucleotide residue in the single-stranded RNA complementary thereto, thereby determining the identity of the nucleotide residue in the single-stranded RNA, and (ii) if no change in hydrogen ion concentration has occurred, iteratively performing step (a), wherein in each iteration of step (a) the dNTP analogue comprises a base which is a different type of base from the type of base of the dNTP analogues in every preceding iteration of step (a), until a dNTP analogue is incorporated into the DNA primer to form a DNA extension product, and determining from the identity of the incorporated dNTP analogue the identity of the nucleotide residue in the single-stranded DNA complementary thereto, thereby determining the identity of the nucleotide residue in the single-stranded DNA.

6. A method for determining the sequence of consecutive nucleotide residues in a single-stranded RNA in a solution comprising:
(a) contacting the single-stranded RNA, having a DNA primer hybridized to a portion thereof, with a reverse transcriptase and a deoxyribonucleotide triphosphate (dNTP) analogue under conditions permitting the reverse transcriptase to catalyze incorporation of the dNTP analogue into the primer if it is complementary to the nucleotide residue of the single-stranded RNA which is immediately 5′ to a nucleotide residue of the single-stranded RNA hybridized to the 3′ terminal nucleotide residue of the DNA primer, so as to form a DNA extension product, wherein (1) the dNTP analogue has the structure: 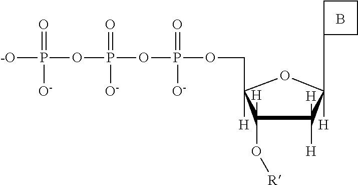
wherein B is a base and is adenine, guanine, cytosine, or thymine, and (2) R′ is (i) —CH₂N₃ or 2-nitrobenzyl, (ii) is a hydrocarbyl, or a substituted hydrocarbyl, having a mass of less than 300 daltons, or (iii) is an dithio moiety;
(b) determining whether incorporation of the dNTP analogue has occurred in step (a) by detecting an increase in hydrogen ion concentration of the solution, wherein an increase in hydrogen ion concentration indicates that the dNTP analogue has been incorporated into the DNA primer to form a DNA extension product, and if so, determining from the identity of the incorporated dNTP analogue the identity of the nucleotide residue in the single-stranded RNA complementary thereto, thereby determining the identity of the nucleotide residue in the single-stranded RNA, and wherein no change in hydrogen ion concentration indicates that the dNTP analogue has not been incorporated into the DNA primer in step (a);
(c) if no change in hydrogen ion concentration has been detected in step (b), iteratively performing steps (a) and (b), wherein in each iteration of step (a) for a given nucleotide residue, the identity of which is being determined, the dNTP analogue comprises a base which is a different type of base from the type of base of the dNTP analogues in every preceding iteration of step (a) for that nucleotide residue, until a dNTP analogue is incorporated into the DNA primer to form a DNA extension product, and determining from the identity of the incorporated dNTP analogue the identity of the nucleotide residue in the single-stranded RNA complementary thereto, thereby determining the identity of the nucleotide residue in the single-stranded RNA;
(d) if an increase in hydrogen ion concentration has been detected and a dNTP analogue is incorporated, subsequently treating the incorporated dNTP nucleotide analogue so as to replace the R′ group thereof with an H atom thereby providing a 3′ OH group at the 3′ terminal of the DNA extension product; and
(e) iteratively performing steps (a) to (d), as necessary, for each nucleotide residue of the consecutive nucleotide residues of the single-stranded RNA to be sequenced, except that in each repeat of step (a) the dNTP analogue is (i) incorporated into the DNA extension product resulting from a preceding iteration of step (a) or step (c), and (ii) complementary to a nucleotide residue of the single-stranded RNA which is immediately 5′ to a nucleotide residue of the single-stranded RNA hybridized to the 3′ terminal nucleotide residue of the DNA extension product resulting from a preceding iteration of step (a) or step (c), so as to form a subsequent DNA extension product, with the proviso that for the last nucleotide residue to be sequenced step (d) is optional,
thereby determining the identity of each of the consecutive nucleotide residues of the single-stranded RNA so as to thereby determine the sequence of the consecutive nucleotide residues of the RNA.

7. The method of any one of claims 1-6, wherein in the dNTP analogue or the rNTP analogue R′ is an alkyldithiomethyl moiety.

8. The method of any one of claims 1-7, wherein for each dNTP analogue or rNTP analogue, R′ is an alkyldithiomethyl that has the structure: 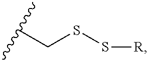
wherein R is the alkyl portion of the alkyldithiomethyl moiety and the wavy line represents the point of connection to the 3′-oxygen.

9. The method of claim 8, wherein the alkyldithiomethyl is independently selected from the group consisting of methyldithiomethyl, ethyldithiomethyl, propyldithiomethyl, isopropyldithiomethyl, butyldithiomethyl, t-butyldithiomethyl, and phenyldithiomethyl.

10. The method of any one of claims 1-9, wherein the RNA is in a solution in a reaction chamber disposed on a sensor which is (i) formed in a semiconductor substrate and (ii) comprises a field-effect transistor or chemical field-effect transistor configured to provide at least one output signal in response to an increase in hydrogen ion concentration of the solution resulting from the formation of a phosphodiester bond between a nucleotide triphosphate or nucleotide triphosphate analogue and a primer or a DNA or RNA extension product.

11. The method of claim 10, wherein the reaction chamber is one of a plurality of reaction chambers disposed on a sensor array formed in a semiconductor substrate and comprised of a plurality of sensors, each reaction chamber being disposed on at least one sensor and each sensor of the array comprising a field-effect transistor, or a chemical field-effect transistor, configured to provide at least one output signal in response to an increase in hydrogen ion concentration of the solution resulting from the formation of a phosphodiester bond between a nucleotide triphosphate or nucleotide triphosphate analogue and a primer or a DNA or RNA extension product.

12. The method of claim 11, wherein said sensors of said array each occupy an area of 100 μm or less and have a pitch of 10 μm or less and wherein each of said reaction chambers has a volume in the range of from 1 μm³ to 1500 μm³; or wherein each of said reaction chambers contains at least 105 copies of the single-stranded DNA or RNA in the solution.

13. The method of claim 11 or 12, wherein said plurality of said reaction chambers and said plurality of said sensors are each greater in number than 256,000.

14. The method of any one of claims 1-13, wherein single-stranded DNA(s) or RNA(s) in the solution are attached to a solid substrate; wherein a primer in the solution is attached to a solid substrate; wherein the single-stranded RNA or primer is attached to a solid substrate via 1,3-dipolar azide-alkyne cycloaddition chemistry; wherein the single-stranded DNA or RNA or primer is attached to a solid substrate via a polyethylene glycol molecule; wherein the single-stranded DNA or RNA or primer is attached to a solid substrate via a polyethylene glycol molecule and is azide-functionalized; wherein the DNA or RNA or primer is attached to a solid substrate via an azido linkage, an alkynyl linkage, or biotin-streptavidin interaction; wherein the DNA or RNA or primer is alkyne-labeled; wherein the DNA or RNA or primer is attached to a solid substrate which is in the form of a chip, a bead, a well, a capillary tube, a slide, a wafer, a filter, a fiber, a porous media, a matrix, a porous nanotube, or a column; wherein the DNA or RNA or primer is attached to a solid substrate which is a metal, gold, silver, quartz, silica, a plastic, polypropylene, a glass, nylon, or diamond; wherein the DNA or RNA or primer is attached to a solid substrate which is a porous non-metal substance to which is attached or impregnated a metal or combination of metals; wherein the DNA or RNA or primer is attached to a solid substrate which is in turn attached to a second solid substrate; or wherein the DNA or RNA or primer is attached to a solid substrate which is in turn attached to a second solid substrate which is a chip.

15. The method of any one of claims 1-14, wherein 1×10⁹ or fewer copies of the DNA or RNA or primer are attached to a solid substrate; wherein 1×10⁸ or fewer copies of the DNA or RNA or primer are attached to a solid substrate; wherein 2×10⁷ or fewer copies of the DNA or RNA or primer are attached to a solid substrate; wherein 1×10⁷ or fewer copies of the DNA or RNA or primer are attached to a solid substrate; wherein 1×10⁶ or fewer copies of the DNA or RNA or primer are attached to a solid substrate; wherein 1×10⁴ or fewer copies of the DNA or RNA or primer are attached to a solid substrate; or wherein 1,000 or fewer copies of the DNA or RNA or primer are attached to a solid substrate.

16. The method of any one of claims 1-14, wherein 10,000 or more copies of the DNA or RNA or primer are attached to a solid substrate; wherein 1×10⁷ or more copies of the DNA or RNA or primer are attached to a solid substrate; wherein 1×10⁸ or more copies of the DNA or RNA or primer are attached to a solid substrate; or wherein 1×10⁹ or more copies of the DNA or RNA or primer are attached to a solid substrate.

17. The method of any one of claims 1-16, wherein the DNA or RNA or primer are separated in discrete compartments, wells, or depressions on a solid surface.

18. The method of any one of claims 1-17 performed in parallel on a plurality of single-stranded DNA(s) or RNAs; and wherein optionally the single-stranded DNAs or RNAs are templates having the same sequence.

19. The method of claim 18, further comprising contacting the plurality of single-stranded DNAs or RNAs or templates after the residue of the nucleotide residue has been determined in step (b), or (c), as appropriate, with a dideoxynucleotide triphosphate which is complementary to the nucleotide residue which has been identified, so as to thereby permanently cap any unextended primers or unextended DNA or RNA extension products.

20. The method of claims 18 or 19, wherein the single-stranded DNA or RNA is amplified from a sample of DNA or RNA prior to step (a); and wherein optionally the single-stranded DNA or RNA is amplified by polymerase chain reaction.

21. The method of any one of claims 1-20, wherein UV light is used to treat the R′ group of a dNTP analogue or rNTP analogue incorporated into a primer or DNA or RNA extension product so as to photochemically cleave the moiety attached to the 3′-O so as to replace the 3‘-O-R’ with a 3′-OH; wherein the moiety is optionally a 2-nitrobenzyl moiety.

22. The method of any one of claims 1-20, wherein tris-(2-carboxyethyl)phosphine (TCEP) or tris(hydroxypropyl)phosphine (THP) is used to treat the R′ group of a dNTP analogue or rNTP analogue incorporated into a primer or DNA or RNA extension product, so as to cleave the moiety attached to the 3′-O so as to replace the 3‘-O-R’ with a 3′-OH; wherein the moiety is optionally a alkyldithiomethyl moiety.

23. The method of claim 22, wherein the alkyldithiomethyl is independently selected from the group consisting of methyldithiomethyl, ethyldithiomethyl, propyldithiomethyl, isopropyldithiomethyl, butyldithiomethyl, t-butyldithiomethyl, and phenyldithiomethyl.

24. The method of any one of claims 1-6 and 10-23 wherein R′ of the dNTP analogue or rNTP analogue comprises a dithio moiety.

25. The method of claim 24, wherein R′ has the structure: 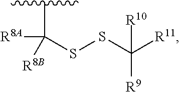
wherein, R^{8A}, R^{8B}, R⁹, R¹⁰, and R¹¹ are each independently hydrogen, CH₃, —CX₃, —CHX₂, —CH₂X, —OCX₃, —OCH₂X, —OCHX₂, —CN, —OH, —SH, —NH₂, a substituted alkyl, a size-limited substituted alkyl, a lower substituent group substituted alkyl, an unsubstituted alkyl, a substituted heteroalkyl, a size-limited substituent group substituted heteroalkyl, a lower substituent group substituted heteroalkyl, an unsubstituted heteroalkyl, a substituted heteroalkyl, a size-limited substituent group substituted heteroalkyl, a lower substituent group substituted heteroalkyl unsubstituted cycloalkyl, a substituted cycloalkyl, a size-limited substituent group substituted cycloalkyl, a lower substituent group substituted cycloalkyl, an unsubstituted heterocycloalkyl, a substituted heterocycloalkyl, a size-limited substituent group substituted heterocycloalkyl, a lower substituent group substituted heterocycloalkyl, an unsubstituted aryl, a substituted aryl, a size-limited substituent group substituted aryl, a lower substituent group substituted aryl or an unsubstituted heteroaryl,
wherein X is independently halogen.
